(19) United States
(12) Patent Application Publication
(10) Pub. No.: US 2006/0276496 A1
(43) Pub. Date: Dec. 7, 2006

(54) Anti-Cytokine Heterocyclic Compounds

(75) Inventors: Daniel Goldberg, Redding, CT (US); Asitha Abeywardane, Danbury, CT (US); Craig Miller, Danbury, CT (US); Tina Morwick, Danbury, CT (US); Matthew Netherton, Danbury, CT (US); Roger Snow, Danbury, CT (US); Ji Wang, Danbury, CT (US); Jiang-Ping Wu, Danbury, CT (US); Zhaoming Xiong, Danbury, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/276,933

(22) Filed: Mar. 17, 2006

Related U.S. Application Data
(60) Provisional application No. 60/719,164, filed on Sep. 21, 2005.
(60) Provisional application No. 60/662,936, filed on Mar. 17, 2005.

(51) Int. Cl. A61K 31/4745; A61K 31/403; C07D 471/02

(52) U.S. Cl. 514/291; 514/411; 548/444; 546/85

(57) ABSTRACT

Heterocyclic compounds and analogues thereof and their use as inhibitors of Mitogen-Activated Protein Kinase-Activated Protein kinase-2 (MAPKAP-k2), and also to a method for preventing or treating a disease or disorder that can be treated or prevented by modulating the activity of MAPKAP-K2 in a subject and to pharmaceutical compositions and kits that include these MAPKAP-K2 inhibitors.

### BACKGROUND OF THE INVENTION

1. Field of the Invention

This present invention relates to heterocyclic compounds and analogues thereof and their use as inhibitors of Mitogen-Activated Protein Kinase-Activated Protein kinase-2 (MAPKAP-k2), and also to a method for preventing or treating a disease or disorder that can be treated or prevented by modulating the activity of MAPKAP-k2 in a subject and to pharmaceutical compositions and kits that include these MAPKAP-K2 inhibitors.

2. Description of Related Art

Mitogen Activated Protein Kinases (MAPKs) are members of signal transduction pathways that change cell physiology in response to external stimuli by activating a variety of downstream signaling genes products. These gene products control diverse cellular functions such as the production of pro-inflammatory cytokines involved in establishing and maintaining specific human diseases. The MAPKs are activated by phosphorylation on specific residues within the activation loop sequence by specific upstream MAPK kinases (MKKs) in response to a cellular activation signal. In turn, the MAPKs activate a variety of downstream gene products. There are four major classes of MAPKs: 1) the archetypal Extracellular Regulated kinases (ERKs), 2) the c-jun N-terminal kinases (JNKs), 3) the p38 MAPKs and finally, 4) the ERK5 or BigMAPKs. The MAPK pathways are involved in alterations in cell physiology resulting from cell stimulation. They control various cell processes such as: cell death, cell cycle machinery, gene transcription and protein translation, (Tibbles and Woodgett; Kyriakis and Avruch).

Of particular relevance to this invention, is the p38 MAPK family (also known as p38, SAPK2a, RK, MPK2, Mxi2 and CSBP). These kinases, most notably the p38alpha and p38beta isoforms, can activate a wide variety of regulatory proteins. In this manner, p38 can diversify downstream signaling leading to a wide variety of cellular outcomes. Central to the signal transduction process initiated by p38 activation is MAPKAP-K2. Most of the physiological outcomes of MAPKAP-K2 have been established using mice genetically deficient in MAPKAP-K2 (designated MAPKAP-K2^{(-/-)}). A significant phenotype of the MAPKAP-K2^{(-/-)} mice is that pro-inflammatory cytokine production is inhibited following stimulation of splenocytes with lipopolysaccharide (LPS). Specifically, the production of tumor necrosis factor-alpha (TNF-alpha) is blocked by 92%, interleukin-1beta (IL-1-beta) is blocked by 40%, IL-6 is blocked by 87% and interferon-gamma (IFN-gamma) is blocked by 86%. This phenotype cannot be rescued by the expression of a kinase dead MAPKAP-K2 mutant, indicating that the kinase function of MAPKAP-K2 is required for proinflammatory cytokine production (Kotlyarov et al.). Thus, an inhibitor of MAPKAP-K2 kinase activity has the potential to exhibit the same inhibitory effects on the production of proinflammatory cytokines.

MK2 activates a number of substrates, including the mRNA binding protein, tristetraproline (TTP). TTP expression is induced by proinflammatory stimuli such as lipopolysaccharide (LPS) or tumor necrosis factor-alpha (TNF-alpha). TTP binds to the AU-rich element within the 3′-untranslated region of the TNF-alpha transcript resulting in a decrease in TNF-alpha mRNA stability (Phillips et al.). TTP^{(-/-)} mice exhibit many defects including arthritis and systemic lupus erythematosis-like symptoms presumably resulting from an increase in circulating TNF-alpha levels (Taylor et al.). Data from in vivo studies with MK2^{(-/-)} indicate that the repressive effects of TTP on both TNF-alpha and interleukin-6 (IL-6) production are downstream of MK2 further establishing p38-MK2-TTP as a critical signaling sequence for the production of proinflammatory cytokines (Neininger et al.).

Elevated levels of proinflammatory cytokines are associated with a number of diseases such as toxic shock syndrome, rheumatoid arthritis, osteoarthritis, diabetes and inflammatory bowel disease (Dinarello). In these diseases, chronic elevation of inflammation exacerbates or causes much of the pathophysiology observed. For example, rheumatoid synovial tissue becomes invaded with inflammatory cells that result in destruction to cartilage and bone (Koch, Kunkel, and Strieter). Studies suggest that inflammatory changes mediated by cytokines may be involved in endothelial cell pathogenesis including restenosis after percutaneous transluminal coronary angioplasty (PTCA) (Tashiro et al.). An important and accepted therapeutic approach for potential drug intervention in these diseases is the reduction of pro-inflammatory cytokines such as TNF-alpha and IL-1-beta. Several biological agents directed against these pro-inflammatory cytokines (anti-TNF antibodies, a soluble TNF receptor and an IL-1 receptor antagonist) have been FDA approved for the treatment of RA, Crohn's disease and psoriatic arthritis (Rankin et al.; Stack et al.; Present et al.; Rutgeerts; Abbott Laboratories markets HUMIRA® (Adalimumab) for the treatment of rheumatoid arthritis (RA); Weinblatt et al.; Jarvis and Faulds; Mease et al.; Nuki et al.).

A soluble TNF-alpha receptor has been engineered that interacts with TNF-alpha. The approach is similar to that described above for the monoclonal antibodies directed against TNF-alpha; both agents bind to soluble TNF-alpha, thus reducing its concentration. One version of this construct, Enbrel® (Immunex, Seattle, Wash.), is marketed for the treatment of rheumatoid arthritis, psoriasis, ankylosing spondylitis, and psoriatic arthritis. Another version of the TNF-alpha receptor, Ro 45-2081 (Hoffman-LaRoche Inc., Nutley, N.J.) has demonstrated efficacy in various animal models of allergic lung inflammation and acute lung injury. Ro 45-2081 is a recombinant chimeric molecule constructed from the soluble 55 kDa human TNF receptor fused to the hinge region of the heavy chain IgG1 gene and expressed in eukaryotic cells (Renzetti and Gater).

Proinflammatory cytokines such as TNF-alpha and IL-6 are also important mediators of septic shock and associated cardiopulmonary dysfunction, acute respiratory distress syndrome (ARDS) and multiple organ failure. In a study of patients presenting with sepsis, a correlation was found between TNF-alpha and IL-6 levels and septic complications (Terregino et al.). TNFα has also been implicated in cachexia and muscle degradation, associated with HIV infection (Lahdevirta et al.). Obesity is associated with an increase incidence of infection, diabetes and cardiovascular disease. Abnormalities in TNF-alpha expression have been noted for each of the above conditions (Loffreda et al.). It has been proposed that elevated levels of TNF-alpha are involved in other eating related disorders such as anorexia and bulimia nervosa. Pathophysiological parallels are drawn between anorexia nervosa and cancer cachexia (Holden and Pakula). An inhibitor of TNF-alpha production, HU-211, was shown to improve the outcome of closed brain injury in an experimental model (Shohami et al.). Atherosclerosis is known to have an inflammatory component and cytokines such as IL-1 and TNF have been suggested to promote the disease. In an animal model an IL-1 receptor antagonist was shown to inhibit fatty streak formation (Elhage et al.).

TNF-alpha levels are elevated in airways of patients with chronic obstructive pulmonary disease and it may contribute to the pathogenesis of this disease (Higham et al.). Circulating TNFα may also contribute to weight loss associated with this disease (Takabatake et al.). Elevated TNF-alpha levels have also been found to be associated with congestive heart failure and the level has been correlated with severity of the disease (Feldman et al.). In addition, TNF-alpha has been implicated in reperfusion injury in lung (Borjesson et al.), kidney (Lemay et al.), and the nervous system (Mitsui et al.). TNF-alpha is also a potent osteoclastogenic agent and is involved in bone resorption and diseases involving bone resorption (bu-Amer et al.). It has also been found highly expressed in chondrocytes of patients with traumatic arthritis (Melchiorri et al.). TNF-alpha has also been shown to play a key role in the development of glomerulonephritis (Le et al.).

The proinflammatory cytokine IL-6 has been implicated with the acute phase response. IL-6 is a growth factor in a number in oncological diseases including multiple myeloma and related plasma cell dyscrasias (Treon and Anderson). It has also been shown to be an important mediator of inflammation within the central nervous system. Elevated levels of IL-6 are found in several neurological disorders including AIDS dementia complex, Alzheimer's disease, multiple sclerosis, systemic lupus erythematosus, CNS trauma and viral and bacterial meningitis (Gruol and Nelson). IL-6 also plays a significant role in osteoporosis. In murine models it has been shown to effect bone resorption and to induce osteoclast activity (Ershler, Harman, and Keller). Marked cytokine differences, such as IL-6 levels, exist in vivo between osteoclasts of normal bone and bone from patients with Paget's disease (Mills and Frausto). A number of cytokines have been shown to be involved in cancer cachexia. The severity of key parameters of cachexia can be reduced by treatment with anti IL-6 antibodies or with IL-6 receptor antagonists (Strassmann and Kambayashi). Several infectious diseases, such as influenza, indicate IL-6 and IFN alpha as key factors in both symptom formation and in host defense (Hayden et al.). Overexpression of IL-6 has been implicated in the pathology of a number of diseases including multiple myeloma, rheumatoid arthritis, Castleman's disease, psoriasis, post-menopausal osteoporosis and juvenile idiopathic arthritis (Simpson et al.; Nishimoto and Kishimoto). Compounds that interfered with the production of cytokines including IL-6, and TNF were effective in blocking a passive cutaneous anaphylaxis in mice (Scholz et al.). More recently, a humanized antibody directed against the IL-6 receptor, demonstrated efficacy in a randomized double-blind pilot human clinical study by significantly reducing the Crohn's disease activity index (Ito et al.).

IFN-gamma has been implicated in a number of diseases. It has been associated with increased collagen deposition that is a central histopathological feature of graft-versus-host disease (Parkman). Following kidney transplantation, a patient was diagnosed with acute myelogenous leukemia. Retrospective analysis of peripheral blood cytokines revealed elevated levels of GM-CSF and IFN-gamma. These elevated levels coincided with a rise in peripheral blood white cell count (Burke et al.). The development of insulin-dependent diabetes (Type 1) can be correlated with the accumulation in pancreatic islet cells of T-cells producing IFN-gamma (Ablamunits et al.). IFN-gamma along with TNF, IL-2 and IL-6 lead to the activation of most peripheral T-cells prior to the development of lesions in the central nervous system for diseases such as multiple sclerosis (MS) and AIDS dementia complex (Martino et al.). Atherosclerotic lesions result in arterial disease that can lead to cardiac and cerebral infarction. Many activated immune cells are present in these lesions, mainly T-cells and macrophages. These cells produce large amounts of proinflammatory cytokines such as TNF, IL-1 and IFN-gamma. These cytokines are thought to be involved in promoting apoptosis or programmed cell death of the surrounding vascular smooth muscle cells resulting in the atherosclerotic lesions (Geng). Allergic subjects produce mRNA specific for IFN-gamma following challenge with Vespula venom (Bonay et al.). The expression of a number of cytokines, including IFN-gamma has been shown to increase following a delayed type hypersensitivity reaction thus indicating a role for IFN-gamma in atopic dermatitis (Szepietowski et al.). Histopathologic and immunohistologic studies were performed in cases of fatal cerebral malaria. Evidence for elevated IFN-gamma amongst other cytokines was observed indicating a role in this disease (Udomsangpetch et al.). The importance of free radical species in the pathogenesis of various infectious diseases has been established. The nitric oxide synthesis pathway is activated in response to infection with certain viruses via the induction of proinflammatory cytokines such as IFN-gamma (Akaike, Suga, and Maeda). Patients, chronically infected with hepatitis B virus (HBV) can develop cirrhosis and hepatocellular carcinoma. Viral gene expression and replication in HBV transgenic mice can be suppressed by a post-transcriptional mechanism mediated by IFN-gamma, TNF and IL-2 (Chisari and Ferrari). IFN-gamma can selectively inhibit cytokine induced bone resorption. It appears to do this via the intermediacy of nitric oxide (NO) which is an important regulatory molecule in bone remodeling. NO may be involved as a mediator of bone disease for such diseases as: rheumatoid arthritis, tumor associated osteolysis and postmenopausal osteoporosis (Evans and Ralston). Studies with gene deficient mice have demonstrated that the IL-12 dependent production of IFN-gamma is critical in the control of early parasitic growth. Although this process is independent of nitric oxide the control of chronic infection does appear to be NO dependent (Alexander et al.). NO is an important vasodilator and convincing evidence exists for its role in cardiovascular shock (Kilbourn, Traber, and Szabo). IFN-gamma is required for progression of chronic intestinal inflammation in such diseases as Crohn's disease and inflammatory bowel disease (IBD) presumably through the intermediacy of CD4+ lymphocytes probably of the TH1 phenotype (Sartor). An elevated level of serum IgE is associated with various atopic diseases such as bronchial asthma and atopic dermatitis. The level of IFN-gamma was negatively correlated with serum IgE suggesting a role for IFNgamma in atopic patients (Teramoto et al.).

The proinflammatory cytokine, IL-1-beta, is partially controlled by MAPKAP-k2. Hence, inhibition of MAPKAP-k2 may impact IL-1-beta dependent processes. IL-1 has been implicated as an immunological effector molecule in a large number of disease processes. IL-1 receptor antagonist (IL-1 ra) had been examined in human clinical trials. Efficacy has been demonstrated for the treatment of rheumatoid arthritis (Antril, Amgen). In a phase III human clinical trial IL-1ra reduced the mortality rate in patients with septic shock syndrome (Dinarello). Several other diseases affected by IL-1 include Adult Onset Still's disease, macrophage auto-activation syndromes, Muckle-Wells syndrome, Familial Cold Autoinflammatory Syndrome and Neonatal Onset Multisystem Inflammatory Disease (Dinarello). Patients with Muckle-Wells syndrome exhibiting systemic inflammation were treated with anakinra (IL-1 ra), leukocytosis serum amyloid A, C-reactive protein and local inflammatory arthritis were reduced with a few days demonstrating that systemic inflammation is IL-1 mediated (Hawkins et al.). Osteoarthritis is a slow progressive disease characterized by destruction of the articular cartilage. IL-1 is detected in synovial fluid and in the cartilage matrix of osteoarthritic joints. Antagonists of IL-1 have been shown to diminish the degradation of cartilage matrix components in a variety of experimental models of arthritis (Chevalier). Nitric oxide (NO) is a mediator of cardiovascular homeostasis, neurotransmission and immune function; recently it has been shown to have important effects in the modulation of bone remodeling. Cytokines such as IL-1 and TNF are potent stimulators of NO production. NO is an important regulatory molecule in bone with effects on cells of the osteoblast and osteoclast lineage (Evans and Ralston). The promotion of beta-cell destruction leading to insulin dependent diabetes mellitus shows dependence on IL-1. Some of this damage may be mediated through other effectors such as prostaglandins and thromboxanes. IL-1 can effect this process by controlling the level of both cyclooxygenase II and inducible nitric oxide synthetase expression (McDaniel et al.).

Inhibitors of cytokine production are expected to block inducible cyclooxygenase (COX-2) expression. COX-2 expression has been shown to be increased by cytokines and it is believed to be the isoform of cyclooxygenase responsible for inflammation (O'Banion, Winn, and Young). Accordingly, inhibitors of MAPKAP-k2 reducing the production of cytokines such as IL-1, would be expected to exhibit efficacy against those disorders currently treated with COX inhibitors such as the familiar NSAIDs. These disorders include acute and chronic pain as well as symptoms of inflammation and cardiovascular disease.

Elevation of several cytokines has been demonstrated during active inflammatory bowel disease (IBD). A mucosal imbalance of intestinal IL-1 and IL-1ra is present in patients with IBD. Insufficient production of endogenous IL-1 ra may contribute to the pathogenesis of IBD (Cominelli and Pizarro). Alzheimer disease is characterized by the presence of beta-amyloid protein deposits, neurofibrillary tangles and cholinergic dysfunction throughout the hippocampal region. The structural and metabolic damage found in Alzheimer disease is possibly due to a sustained elevation of IL-1 (Holden and Mooney). A role for IL-1 in the pathogenesis of human immunodeficiency virus (HIV) has been identified. IL-1 ra showed a clear relationship to acute inflammatory events as well as to the different disease stages in the pathophysiology of HIV infection (Kreuzer et al.). IL-1 and TNF are both involved in periodontal disease. The destructive process associated with periodontal disease may be due to a disregulation of both IL-1 and TNF (Howells).

IL-1 has also been shown to induce uveitis in rats which could be inhibited with IL-1 blockers (Xuan et al.). Cytokines including IL-1, TNF and GM-CSF have been shown to stimulate proliferation of acute myelogenous leukemia blasts (Bruserud). IL-1 was shown to be essential for the development of both irritant and allergic contact dermatitis. Epicutaneous sensitization can be prevented by the administration of an anti-IL-1 monoclonal antibody before epicutaneous application of an allergen (Muller, Knop, and Enk). Data obtained from IL-1 knock out mice indicates the critical involvement in fever for this cytokine (Kluger et al.). A variety of cytokines including TNF, IL-1, IL-6 and IL-8 initiate the acute-phase reaction which is stereotyped in fever, malaise, myalgia, headaches, cellular hypermetabolism and multiple endocrine and enzyme responses (Beisel). The production of these inflammatory cytokines rapidly follows trauma or pathogenic organism invasion.

This patent discloses compounds that have the ability to inhibit TNF-alpha. Compounds disclosed herein are indicated to be effective in treating the following diseases: Rheumatoid arthiritis, psoriasis, crohn's disease, dementia associated with HIV infection, glaucoma, optic-neuropathy, optic neuritis, retinal ischemia, laser induced optic damage, surgery or trauma-induced proliferative vitreoretinopathy, cerebral ischemia, hypoxia-ischemia, hypoglycemia, domoic acid poisoning, anoxia, carbon monoxide or manganese or cyanide poisoning, Huntington's disease, Alzheimer's disease, Parkinson's disease, meningitis, multiple sclerosis and other demyelinating diseases, amyotrophic lateral sclerosis, head and spinal cord trauma, seizures, convulsions, olivopontocerebellar atrophy, neuropathic pain syndromes, diabetic neuropathy, HIV-related neuropathy, MERRF and MELAS syndromes, Leber's disease, Wernicke's encephalophathy, Rett syndrome, homocysteinuria, hyperprolinemia, hyperhomocysteinemia, nonketotic hyperglycinemia, hydroxybutyric aminoaciduria, sulfite oxidase deficiency, combined systems disease, lead encephalopathy, Tourett's syndrome, hepatic encephalopathy, drug addiction, drug tolerance, drug dependency, depression, anxiety and schizophrenia. In addition, compounds dislosed herein are useful for treating acute and chronic inflammation in the lung caused by inhalation of smoke such as cigarette smoke. TNF-alpha anatagonists are apparently also useful for the treatment of endometriosis, see EP 1022027 A1. Infliximab® of rheumatoid arthritis, psoriasis, ankylosing spondylitis, and psoriatic arthritis. The p38MAP kinase pathway plays an role in B.burgdorferi-elicited infammation and may be useful in treating inflammation induced by the Lyme disease agent. Anguita, J. et. al., The Journal of Immunology, 2002, 168:6352-6357.

The work cited above supports the principle that inhibition of cytokine production will be beneficial in the treatment of cytokine mediated diseases. Therefore a need exists for small molecule inhibitors for treating these diseases with optimized efficacy, pharmacokinetic and safety profiles.

### REFERENCE LIST

Abbott Laboratories receives FDA approval earlier than anticipated for HUMIRA (Adalimumab) for the treatment of rheumatoid arthritis (RA). Company Press Release (Abbott Lab.), Dec. 31, 2002 (2002).
Ablamunits, V., et al. “Islet T cells secreting IFN-gamma in NOD mouse diabetes: arrest by p277 peptide treatment.” J Autoimmun. 11.1 (1998): 73-81.
Akaike, T., M. Suga, and H. Maeda. “Free radicals in viral pathogenesis: molecular mechanisms involving superoxide and NO.” Proc. Soc. Exp Biol Med 217.1 (1998): 64-73.
Alexander, J., et al. “Mechanisms of innate resistance to Toxoplasma gondii infection.” Philos. Trans. R. Soc. Lond B Biol Sci. 352.1359 (1997): 1355-59.
Beisel, W. R. “Herman Award Lecture, 1995: infection-induced malnutrition—from cholera to cytokines.” Am J Clin Nutr. 62.4 (1995): 813-19.
Bonay, M., et al. “Characterization of proliferative responses and cytokine mRNA profiles induced by Vespula venom in patients with severe reactions to wasp stings.” Clin Exp Immunol 109.2 (1997): 342-50.
Borjesson, A., et al. “TNF-alpha stimulates alveolar liquid clearance during intestinal ischemia-reperfusion in rats.” Am J Physiol Lung Cell Mol Physiol 278.1 (2000): L3-12.
Bruserud, O. “Effects of endogenous interleukin 1 on blast cells derived from acute myelogenous leukemia patients.” Leuk. Res 20.1 (1996): 65-73.
bu-Amer, Y., et al. “Tumor necrosis factor receptors types 1 and 2 differentially regulate osteoclastogenesis.” J Biol. Chem. 275.35 (2000): 27307-10.
Burke, G. W., et al. “Early development of acute myelogenous leukemia following kidney transplantation: possible role of multiple serum cytokines.” Leuk. Lymphoma 19.1-2 (1995): 173-80.
Chevalier, X. “Upregulation of enzymatic activity by interleukin-1 in osteoarthritis.” Biomed. Pharmacother. 51.2 (1997): 58-62.
Chisari, F. V. and C. Ferrari. “Hepatitis B virus immunopathology.” Springer Semin. Immunopathol. 17.2-3 (1995): 261-81.
Cominelli, F. and T. T. Pizarro. “Interleukin-1 and interleukin-1 receptor antagonist in inflammatory bowel disease.” Aliment. Pharmacol Ther 10 Suppl 2 (1996): 49-53.
Dinarello, C. A. “Interleukin-1 and interleukin-1 receptor antagonist.” Nutrition 11.5 Suppl (1995): 492-94.
- - - . “Interleukin-1.” Rev. Infect. Dis. 6.1 (1984): 51-95.
- - - . “Therapeutic strategies to reduce IL-1 activity in treating local and systemic inflammation.” Curr. Opin. Pharmacol. 4.4 (2004): 378-85.
Elhage, R., et al. “Differential effects of interleukin-1 receptor antagonist and tumor necrosis factor binding protein on fatty-streak formation in apolipoprotein E-deficient mice.” Circulation 97.3 (1998): 242-44.
Ershler, W. B., S. M. Harman, and E. T. Keller. “Immunologic aspects of osteoporosis.” Dev. Comp Immunol 21.6 (1997): 487-99.
Evans, D. M. and S. H. Ralston. “Nitric oxide and bone.” J Bone Miner. Res 11.3 (1996): 300-05.
Feldman, A. M., et al. “The role of tumor necrosis factor in the pathophysiology of heart failure.” J Am Coll. Cardiol. 35.3 (2000): 537-44.
Geng, Y. J. “Regulation of programmed cell death or apoptosis in atherosclerosis.” Heart Vessels Suppl 12 (1997): 76-80.
Gruol, D. L. and T. E. Nelson. “Physiological and pathological roles of interleukin-6 in the central nervous system.” Mol Neurobiol. 15.3 (1997): 307-39.
Hawkins, P. N., et al. “Spectrum of clinical features in Muckle-Wells syndrome and response to anakinra.” Arthritis Rheum. 50.2 (2004): 607-12.
Hayden, F. G., et al. “Local and systemic cytokine responses during experimental human influenza A virus infection. Relation to symptom formation and host defense.” J Clin Invest 101.3 (1998): 643-49.
Higham, M. A., et al. “Tumour necrosis factor-alpha gene promoter polymorphism in chronic obstructive pulmonary disease.” Eur Respir J 15.2 (2000): 281-84.
Holden, R. J. and P. A. Mooney. “Interleukin-1 beta: a common cause of Alzheimer's disease and diabetes mellitus.” Med Hypotheses 45.6 (1995): 559-71.
Holden, R. J. and I. S. Pakula. “The role of tumor necrosis factor-alpha in the pathogenesis of anorexia and bulimia nervosa, cancer cachexia and obesity.” Med Hypotheses 47.6 (1996): 423-38.
Howells, G. L. “Cytokine networks in destructive periodontal disease.” Oral Dis. 1.4 (1995): 266-70.
Ihle, J. N. “The challenges of translating knockout phenotypes into gene function.” Cell 102.2 (2000): 131-34.
Ito, H., et al. “A pilot randomized trial of a human anti-interleukin-6 receptor monoclonal antibody in active Crohn's disease.” Gastroenterology 126.4 (2004): 989-96.
Jaffray, C., J. Yang, and J. Norman. “Elastase mimics pancreatitis-induced hepatic injury via inflammatory mediators.” J. Surg. Res 90.2 (2000): 95-101.
Jarvis, B. and D. Faulds. “Etanercept: a review of its use in rheumatoid arthritis.” Drugs 57.6 (1999): 945-66.
Kilbourn, R. G., D. L. Traber, and C. Szabo. “Nitric oxide and shock.” Dis. Mon. 43.5 (1997): 277-348.
Kluger, M. J., et al. “The use of knockout mice to understand the role of cytokines in fever.” Clin Exp Pharmacol Physiol 25.2 (1998): 141-44.
Koch, A. E., S. L. Kunkel, and R. M. Strieter. “Cytokines in rheumatoid arthritis.” J. Investig. Med. 43.1 (1995): 28-38.
Kotlyarov, A., et al. “MAPKAP kinase 2 is essential for LPS-induced TNF-alpha biosynthesis.” Nat. Cell Biol. 1.2 (1999): 94-97.
Kotlyarov, A., et al. “Distinct cellular functions of MAPKAP-K2.” Mol. Cell Biol. 22.13 (2002): 4827-35.
Kreuzer, K. A., et al. “The IL-1 system in HIV infection: peripheral concentrations of IL-1 beta, IL-1 receptor antagonist and soluble IL-1 receptor type II.” Clin Exp Immunol 109.1 (1997): 54-58.
Kyriakis, J. M. and J. Avruch. “Mammalian mitogen-activated protein kinase signal transduction pathways activated by stress and inflammation.” Physiol Rev. 81.2 (2001): 807-69.
Lahdevirta, J., et al. “Elevated levels of circulating cachectin/tumor necrosis factor in patients with acquired immunodeficiency syndrome.” Am J Med 85.3 (1988): 289-91.
Le, Hir M., et al. “Prevention of crescentic glomerulonephritis induced by anti-glomerular membrane antibody in tumor necrosis factor-deficient mice.” Lab Invest 78.12 (1998): 1625-31.
Lemay, S., et al. “Prominent and sustained up-regulation of gp130-signaling cytokines and the chemokine MIP-2 in murine renal ischemia-reperfusion injury.” Transplantation 69.5 (2000): 959-63.
Loffreda, S., et al. “Leptin regulates proinflammatory immune responses.” FASEB J 12.1 (1998): 57-65.
Martino, G., et al. “Proinflammatory cytokines regulate antigen-independent T-cell activation by two separate calcium-signaling pathways in multiple sclerosis patients.” Ann. Neurol 43.3 (1998): 340-49.
McDaniel, M. L., et al. “Cytokines and nitric oxide in islet inflammation and diabetes.” Proc. Soc. Exp Biol Med 211.1 (1996): 24-32.
Mease, P. J., et al. “Etanercept in the treatment of psoriatic arthritis and psoriasis: a randomised trial.” Lancet 356.9227 (2000): 385-90.
Melchiorri, C., et al. “Enhanced and coordinated in vivo expression of inflammatory cytokines and nitric oxide synthase by chondrocytes from patients with osteoarthritis.” Arthritis Rheum. 41.12 (1998): 2165-74.
Mills, B. G. and A. Frausto. “Cytokines expressed in multinucleated cells: Paget's disease and giant cell tumors versus normal bone.” Calcif. Tissue Int 61.1 (1997): 16-21.
Mitsui, Y., et al. “The expression of proinflammatory cytokine mRNA in the sciatic-tibial nerve of ischemia-reperfusion injury.” Brain Res 844.1-2 (1999): 192-95.
Muller, G., J. Knop, and A. H. Enk. “Is cytokine expression responsible for differences between allergens and irritants?” Am J Contact Dermat. 7.3 (1996): 177-84.
Neininger, A., et al. “MAPKAP-K2 targets AU-rich elements and regulates biosynthesis of tumor necrosis factor and interleukin-6 independently at different post-transcriptional levels.” J. Biol. Chem. 277.5 (2002): 3065-68.
Nishimoto, N. and T. Kishimoto. “Inhibition of IL-6 for the treatment of inflammatory diseases.” Curr. Opin. Pharmacol. 4.4 (2004): 386-91.
Nuki, G., et al. “Long-term safety and maintenance of clinical improvement following treatment with anakinra (recombinant human interleukin-1 receptor antagonist) in patients with rheumatoid arthritis: extension phase of a randomized, double-blind, placebo-controlled trial.” Arthritis Rheum. 46.11 (2002): 2838-46.
O'Banion, M. K., V. D. Winn, and D. A. Young. “cDNA cloning and functional activity of a glucocorticoid-regulated inflammatory cyclooxygenase.” Proc. Natl. Acad. Sci. U.S.A 89.11 (1992): 4888-92.
Parkman, R. “Chronic graft-versus-host disease.” Curr. Opin. Hematol. 5.1 (1998): 22-25.
Phillips, K., et al. “Arthritis suppressor genes TIA-1 and TTP dampen the expression of tumor necrosis factor alpha, cyclooxygenase 2, and inflammatory arthritis.” Proc. Natl. Acad. Sci. U.S.A 101.7 (2004): 2011-16.
Present, D. H., et al. “Infliximab for the treatment of fistulas in patients with Crohn's disease.” N. Engl. J. Med. 340.18 (1999): 1398-405.
Rankin, E. C., et al. “The therapeutic effects of an engineered human anti-tumour necrosis factor alpha antibody (CDP571) in rheumatoid arthritis.” Br. J. Rheumatol. 34.4 (1995): 334-42.
Renzetti, L. M. and P. R. Gater. “Ro 45-2081, a TNF receptor fusion protein, prevents inflammatory responses in the airways.” Inflamm. Res. 46 Suppl 2 (1997): S143-S144.
Rutgeerts, P. J. “Review article: efficacy of infliximab in Crohn's disease-induction and maintenance of remission.” Aliment. Pharmacol. Ther. 13 Suppl 4 (1999): 9-15.
Sartor, R. B. “Cytokine regulation of experimental intestinal inflammation in genetically engineered and T-lymphocyte reconstituted rodents.” Aliment. Pharmacol Ther 10 Suppl 2 (1996): 36-42.
Scholz, D., et al. “Inhibition of Fc epsilon RI-mediated activation of mast cells by 2,3,4-trihydropyrimidino[2,1-a]isoquinolines.” J Med. Chem. 41.7 (1998): 1050-59.
Shohami, E., et al. “Cytokine production in the brain following closed head injury: dexanabinol (HU-211) is a novel TNF-alpha inhibitor and an effective neuroprotectant.” J Neuroimmunol. 72.2 (1997): 169-77.
Simpson, R. J., et al. “Interleukin-6: structure-function relationships.” Protein Sci. 6.5 (1997): 929-55.
Stack, W. A., et al. “Randomised controlled trial of CDP571 antibody to tumour necrosis factor-alpha in Crohn's disease.” Lancet 349.9051 (1997): 521-24.
Strassmann, G. and T. Kambayashi. “Inhibition of experimental cancer cachexia by anti-cytokine and anti-cytokine-receptor therapy.” Cytokines Mol Ther 1.2 (1995): 107-13.
Szepietowski, J. C., et al. “Atopic and non-atopic individuals react to nickel challenge in a similar way. A study of the cytokine profile in nickel-induced contact dermatitis.” Br J. Dermatol. 137.2 (1997): 195-200.
Takabatake, N., et al. “The relationship between chronic hypoxemia and activation of the tumor necrosis factor-alpha system in patients with chronic obstructive pulmonary disease.” Am J Respir Crit Care Med 161.4 Pt 1 (2000): 1179-84.
Tamura, K., et al. “Requirement for p38alpha in erythropoietin expression: a role for stress kinases in erythropoiesis.” Cell 102.2 (2000): 221-31.
Tashiro, H., et al. “Role of cytokines in the pathogenesis of restenosis after percutaneous transluminal coronary angioplasty.” Coron. Artery Dis. 12.2 (2001): 107-13.
Taylor, G. A., et al. “A pathogenetic role for TNF alpha in the syndrome of cachexia, arthritis, and autoimmunity resulting from tristetraprolin (TTP) deficiency.” Immunity. 4.5 (1996): 445-54.
Teramoto, T., et al. “Serum IgE level is negatively correlated with the ability of peripheral mononuclear cells to produce interferon gamma (IFNgamma): evidence of reduced expression of IFNgamma mRNA in atopic patients.” Clin Exp Allergy 28.1 (1998): 74-82.
Terregino, C. A., et al. “Endogenous mediators in emergency department patients with presumed sepsis: are levels associated with progression to severe sepsis and death?” Ann. Emerg. Med 35.1 (2000): 26-34.
Tibbles, L. A. and J. R. Woodgett. “The stress-activated protein kinase pathways.” Cell. Mol. Life Sci. 55.10 (1999): 1230-54.
Treon, S. P. and K. C. Anderson. “Interleukin-6 in multiple myeloma and related plasma cell dyscrasias.” Curr. Opin. Hematol. 5.1 (1998): 42-48.
Udomsangpetch, R., et al. “Involvement of cytokines in the histopathology of cerebral malaria.” Am J Trop. Med Hyg. 57.5 (1997): 501-06.
Weinblatt, M. E., et al. “A trial of etanercept, a recombinant tumor necrosis factor receptor:Fc fusion protein, in patients with rheumatoid arthritis receiving methotrexate.” N. Engl. J. Med. 340.4 (1999): 253-59.
- - - . “Anti-tumor necrosis factor ameliorates joint disease in murine collagen-induced arthritis.” Proc. Natl. Acad. Sci. U.S.A 89.20 (1992): 9784-88.
Xuan, B., et al. “Effective treatment of experimental uveitis with interleukin-1 blockers, CK 123 and CK 124.” J Ocul. Pharmacol Ther 14.1 (1998): 31-44.

### BRIEF SUMMARY OF THE INVENTION

The work cited above supports the principle that inhibition of MAPKAP-k2 will be beneficial in the treatment of various disease states.

It is therefore an object of the invention to provide compounds of formulas I and IA: 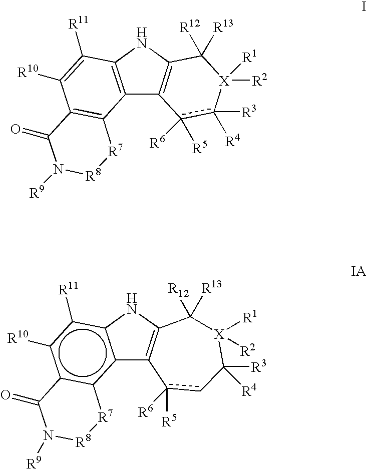
wherein X and R¹-R¹³ of formulas (I) and (IA) are defined below, which inhibit the activity of MAPKAP-k2.

It is a further object of the invention to provide methods for treating MAPKAP-k2 mediated diseases and pathological conditions involving inflammation such as chronic inflammatory disease, using the novel compounds of the invention.

### DETAILED SUMMARY OF THE INVENTION

It is yet a further object of the invention to provide processes of preparation of the above-mentioned novel compounds.

In an embodiment of the present invention, there are provided compounds of the formula I: 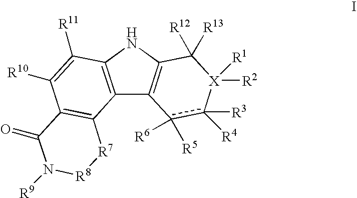
wherein:
X is C or N
R¹ is hydrogen, hydroxy, carbamoyl, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆alkylthio C₁₋₆alkyl, wherein the sulfur atom, when present, is optionally oxidized to a sulfoxide or sulfone;
R² is optionally present and, if present, is hydrogen, hydroxy, ureido, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆ alkylthio, wherein the sulfur atom, when present, is optionally oxidized to a sulfoxide or sulfone;
R³ is hydrogen, amino, C₁₋₆ alkyl, C₂₋₆ alkenyl, amino C₁₋₆ alkyl, hydroxyC₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbamoyl, CO₂C₁₋₆alkyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x} wherein R^{x} can be chosen from:
   hydrogen, amino, C₁₋₆alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, —CO₂C₁₋₆alkyl or C₂₋₆ alkylthio wherein the sulfur atom is oxidized to a sulfoxide or sulfone;
R⁴ is optionally present and, when present, is hydrogen, amino, C₁₋₆ alkyl, C₂₋₆ alkenyl, amino C₁₋₆ alkyl, hydroxy C₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbonyl, carbamoyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x}, and wherein R^{x} can be chosen from:
   amino, C₁₋₆ alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, or C₂₋₆ alkylthio wherein the sulfur atom is oxidized to a sulfoxide or sulfone;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, C₃₋₇ cycloalkyl, heterocyclyl, heterocyclylC₁₋₆alkyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted by R^{x}, which is defined hereinabove, or ═N—OH, ═N—NHheteroaryl;
R⁴ and R⁵, along with their substituents, may optionally be taken together to form a C₃₋₈ cycloalkyl optionally substituted with amino, C₁₋₃ alkyl, halogen, or hydroxy;
R⁶ is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted with single or multiple R^{x}, which is defined hereinabove, or
R⁵ and R⁶, along with their substituents, may optionally be taken together to form a C₃₋₇ cycloalkyl that may be optionally substituted with single or multiple R^{x}, which is defined hereinabove;
R⁷ is selected from hydrogen, C₁₋₆ alkyl, C₃₋₇ cycloalkyl, C₁₋₆ alkoxy, C₁₋₆alkoxyC₁₋₆alkyl, hydroxy, hydroxy C₁₋₆ alkyl or amino optionally mono- or di-substituted by C₁₋₆ alkyl, aryl or aryl C₁₋₆alkyl, or R⁷ may be halo, haloalkyl, alkoxy, carboxyl, carbamoyl, nitro, dialkylamino, benzyloxy, hydrazinocarbonyl, alkoxycarbonyl, alkoxycarbonylheterocyclcarbonyl, or hydroxy; wherein each alkyl, cycloalkyl, alkenyl, aryl or heteroaryl is optionally substituted with single or multiple R^{x}, which is defined hereinabove,
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl or C₂₋₆ alkylthio, wherein the sulfur atom is optionally oxidized to a sulfoxide or sulfone; wherein each aryl, heterocycle or heteroaryl may be singly or multiply substituted with R^{y}, wherein R^{y} may be chosen from:
   halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, C₂₋₆ alkylthio wherein the sulfur atom is optionally oxidized to a sulfoxide or sulfone, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH, —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino; or
R⁸ and R⁹ can be taken together to form a cycloalkyl or heteraryl ring, with the proviso that it can not be morpholine;
R¹⁰ and R¹¹ are independently selected from hydrogen, C₁₋₆ alkoxy, hydroxy, halo, C₁₋₆ alkyl, C₃₋₇ cycloalkyl;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆ alkyl,
R¹³ is optionally present and, if present, is selected from from hydrogen, C₁₋₆ alkyl, or a halo group.
In a second embodiment there are provided compounds of formula (I) as described above and wherein
X is C or N;
R¹ is hydrogen;
R² is optionally present and, if present, is hydrogen;
   R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
      hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
   R⁴ is optionally present and, when present, is hydrogen or C₁₋₆alkyl;
   R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
   R⁶ is hydrogen or C₁₋₆ alkyl, optionally partially or fully halogenated, or
   R⁵ and R⁶, may optionally be taken together to form a C₃₋₇ cycloalkyl;
   R⁷ is hydrogen, hydroxyl, or C₁₋₆ alkoxy;
   R⁸ is chosen from hydrogen, C₁₋₆ alkyl, C₂₋₆ alkenyl, C₂₋₆ alkynyl, C₁₋₆ alkoxy, or C₃₋₇ cycloalkyl;
   R⁹ is chosen from hydrogen, C₁₋₆ alkyl, piperidinyl, phenyl and heteroaryl, wherein each phenyl, piperidinyl or heteroaryl may be singly or multiply substituted with R^{y}, wherein R^{y} may be chosen from:
      halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
      R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
   R¹⁰ and R¹¹ are hydrogen;
   R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
   R¹³ is optionally present and, if present, is selected from from hydrogen, C₁₋₆ alkyl, or a halo group.
   In another embodiment there are provided compounds of formula (I) as described above and wherein
   X is C or N;
   R¹ is hydrogen;
   R² is optionally present and, if present, is hydrogen;
   R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
      hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆alkoxy, C₂₋₆alkenyloxy, C₂₋₆alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
   R⁴ is optionally present and, when present, is hydrogen or C₁₋₆alkyl;
   R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
   R⁶ is hydrogen or C₁₋₆ alkyl, optionally partially or fully halogenated, or
   R⁵ and R⁶, may optionally be taken together to form a C₃₋₇ cycloalkyl;
   R⁷ is hydrogen, hydroxyl, or C₁₋₆ alkoxy;
   R⁸ is chosen from hydrogen, C₁₋₆ alkyl, C₂₋₆ alkenyl, C₂₋₆ alkynyl, C₁₋₆ alkoxy, or C₃₋₇ cycloalkyl;
   R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran wherein each phenyl, piperidinyl or heteroaryl may be singly or multiply substituted with R^{y}, wherein R^{y} may be chosen from:
      halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
   R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
   R¹⁰ and R¹¹ are hydrogen;
   R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
   R¹³ is optionally present and, if present, is selected from from hydrogen, C₁₋₆ alkyl, or a halo group.

In yet another embodiment there are provided compounds of formula (I) as described above and wherein
X is N;
R¹ is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
   hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is selected from hydrogen, or C₁₋₆alkyl;
R⁵ is optionally present and, when present, is hydrogen, C₁₋₆ alkyl or CF₃;
R⁶ is hydrogen, C₁₋₆ alkyl or CF₃; or
R⁵ and R⁶, may optionally be taken together to form a C₃₋₇ cycloalkyl;
R⁷ is hydrogen;
R⁸ is hydrogen;
R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran, wherein each phenyl, piperidinyl or heteroaryl may be singly or multiply substituted with R^{y}, wherein R^{y} may be chosen from:
   halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂ or —C(O)N(R^{a})(R^{b}) where R^{a} and R^{b} are independently selected from
   H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, and —C(O)NH—N═C—R^{e}, where R^{e} is phenyl, or
   R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen; and
R¹² is oxo.

In a further embodiment, of the invention, there are provided compounds of the formula Ia: 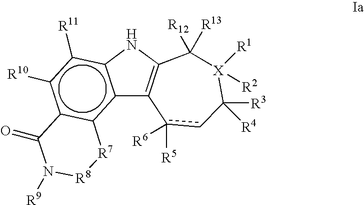
wherein:
X is C or N
R¹ is hydrogen, hydroxy, carbamoyl, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆alkylthio C₁₋₆alkyl, wherein the sulfur atom, when present, is optionally oxidized to a sulfoxide or sulfone;
R² is optionally present and, if present, is hydrogen, hydroxy, ureido, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆ alkylthio, wherein the sulfur atom, when present, is optionally oxidized to a sulfoxide or sulfone;
R³ is hydrogen, amino, C₁₋₆ alkyl, C₂₋₆ alkenyl, amino C₁₋₆ alkyl, hydroxyC₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbamoyl, CO₂C₁₋₆alkyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x} wherein R^{x} can be chosen from:
   hydrogen, amino, C₁₋₆alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, —CO₂C₁₋₆alkyl or C₂₋₆ alkylthio wherein the sulfur atom is oxidized to a sulfoxide or sulfone;
R⁴ is optionally present and, when present, is hydrogen, amino, C₁₋₆ alkyl, C₂₋₆ alkenyl, amino C₁₋₆ alkyl, hydroxy C₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbonyl, carbamoyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x}, and wherein R^{x} can be chosen from:
   amino, C₁₋₆ alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, or C₂₋₆ alkylthio wherein the sulfur atom is oxidized to a sulfoxide or sulfone;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, C₃₋₇ cycloalkyl, heterocyclyl, heterocyclylC₁₋₆alkyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted by R^{x}, which is defined hereinabove, or ═N—OH, ═N—NHheteroaryl;
R⁴ and R⁵, along with their substituents, may optionally be taken together to form a C₃₋₈ cycloalkyl optionally substituted with amino, C₁₋₃ alkyl, halogen, or hydroxy;
R⁶ is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted with single or multiple R^{x}, which is defined hereinabove, or
R⁵ and R⁶, along with their substituents, may optionally be taken together to form a C₃₋₇ cycloalkyl that may be optionally substituted with single or multiple R^{x}, which is defined hereinabove;
R⁷ is selected from hydrogen, C₁₋₆ alkyl, C₃₋₇ cycloalkyl, C₁₋₆ alkoxy, C1-6alkoxyC1-6 alkyl, hydroxy, hydroxy C₁₋₆ alkyl or amino optionally mono- or di-substituted by C₁₋₆ alkyl, aryl or aryl C1-6 alkyl, or R⁷ may be halo, haloalkyl, alkoxy, carboxyl, carbamoyl, nitro, dialkylamino, benzyloxy, hydrazinocarbonyl, alkoxycarbonyl, alkoxycarbonylheterocyclcarbonyl, or hydroxy; wherein each alkyl, cycloalkyl, alkenyl, aryl or heteroaryl is optionally substituted with single or multiple R^{x}, which is defined hereinabove,
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₁₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl or C₂₋₆ alkylthio, wherein the sulfur atom is optionally oxidized to a sulfoxide or sulfone; wherein each aryl, heterocycle or heteroaryl may be singly or multiply substituted with R^{y}, wherein R^{y} may be chosen from:
   halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, C₂₋₆ alkylthio wherein the sulfur atom is optionally oxidized to a sulfoxide or sulfone, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH, —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
   R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino; or
R⁸ and R⁹ can be taken together to form a cycloalkyl or heteraryl ring, with the proviso that it can not be morpholine;
R¹⁰ and R¹¹ are independently selected from hydrogen, C₁₋₆ alkoxy, hydroxy, halo, C₁₋₆ alkyl, C₃₋₇ cycloalkyl;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆ alkyl,
R¹³ is optionally present and, if present, is selected from from hydrogen, C₁₋₆ alkyl, or a halo group.
In a second embodiment there are provided compounds of formula (I) as described above and wherein
X is C or N;
R¹ is hydrogen;
R² is optionally present and, if present, is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
   hydrogen, amino, C₁₋₆ alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is hydrogen or C₁₋₆alkyl;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
R⁶ is hydrogen or C₁₋₆ alkyl, optionally partially or fully halogenated, or
R⁵ and R⁶, may optionally be taken together to form a C₃₋₇ cycloalkyl;
R⁷ is hydrogen, hydroxyl, or C₁₋₆ alkoxy;
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, C₁₋₆alkyl, piperidinyl, phenyl and heteroaryl, wherein each phenyl, piperidinyl or heteroaryl may be singly or multiply substituted with R^{y}, wherein R^{y} may be chosen from:
   halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
   R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
R¹³ is optionally present and, if present, is selected from from hydrogen, C₁₋₆ alkyl, or a halo group.
In another embodiment there are provided compounds of formula (I) as described above and wherein
X is C or N;
R¹ is hydrogen;
R² is optionally present and, if present, is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
   hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is hydrogen or C₁₋₆alkyl;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
R⁶ is hydrogen or C₁₋₆ alkyl, optionally partially or fully halogenated, or
R⁵ and R⁶, may optionally be taken together to form a C₃₋₇ cycloalkyl;
R⁷ is hydrogen, hydroxyl, or C₁₋₆ alkoxy;
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran wherein each phenyl, piperidinyl or heteroaryl may be singly or multiply substituted with R^{y}, wherein R^{y} may be chosen from:
   halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
   R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
R¹³ is optionally present and, if present, is selected from from hydrogen, C₁₋₆ alkyl, or a halo group.

In yet another embodiment there are provided compounds of formula (I) as described above and wherein
X is N;
R¹ is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
   hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆alkoxy, C₂₋₆alkenyloxy, C₂₋₆alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is selected from hydrogen, or C₁₋₆alkyl;
R⁵ is optionally present and, when present, is hydrogen, C₁₋₆ alkyl or CF₃;
R⁶ is hydrogen, C₁₋₆ alkyl or CF₃; or R⁵ and R⁶, may optionally be taken together to form a C₃₋₇ cycloalkyl;
R⁷ is hydrogen;
R⁸ is hydrogen;
R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran, wherein each phenyl, piperidinyl or heteroaryl may be singly or multiply substituted with R^{y}, wherein R^{y} may be chosen from:
   halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂ or —C(O)N(R^{a})(R^{b}) where R^{a} and R^{b} are independently selected from
   H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, and —C(O)NH—N═C—R^{e}, where R^{e} is phenyl, or
   R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen; and
R¹² is oxo.

The following are representative examples of the invention which can be made according to the general schemes and working examples below:

TABLE 1
Compound Structures and Names

| | |
|---|---|
| 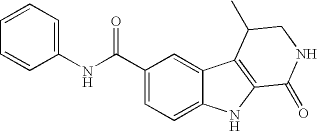 | 4-Methyl-1-oxo-2,3,4,9- tetrahydro-1H-β-carboline-6- carboxylic acid phenylamide |
| 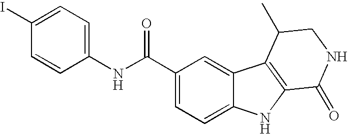 | 4-Methyl-1-oxo-2,3,4,9- tetrahydro-1H-β-carboline-6- carboxylic acid (4-iodo-phenyl)- amide |
| 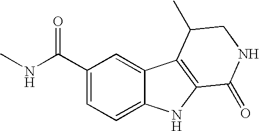 | 4-Methyl-1-oxo-2,3,4,9- tetrahydro-1H-b-carboline-6- carboxylic acid methylamide |
| 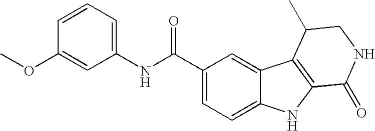 | 4-Methyl-1-oxo-2,3,4,9- tetrahydro-1H-β-carboline-6- carboxylic acid (3-methoxy- phenyl)-amide |
| 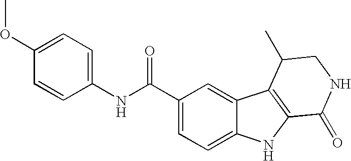 | 4-Methyl-1-oxo-2,3,4,9- tetrahydro-1H-β-carboline-6- carboxylic acid (4-methoxy- phenyl)-amide |
| 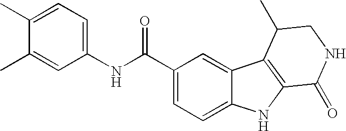 | 4-Methyl-1-oxo-2,3,4,9- tetrahydro-1H-β-carboline-6- carboxylic acid (3,4-dimethyl- phenyl)-amide |
| 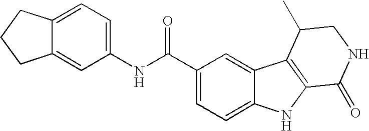 | 4-Methyl-1-oxo-2,3,4,9- tetrahydro-1H-β-carboline-6- carboxylic acid indan-5-ylamide |
| 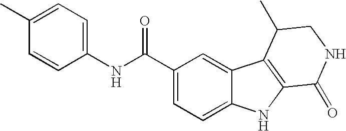 | 4-Methyl-1-oxo-2,3,4,9- tetrahydro-1H-β-carboline-6- carboxylic acid p-tolylamide |
| 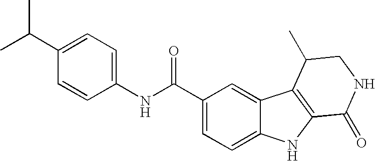 | 4-Methyl-1-oxo-2,3,4,9- tetrahydro-1H-β-carboline-6- carboxylic acid (4-isopropyl- phenyl)-amide |
| 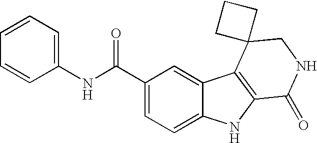 | 4,4-spirocyclobutyl-1-oxo-2,3,4,9- tetrahydro-1H-β-carboline-6- carboxylic acid phenylamide |
| 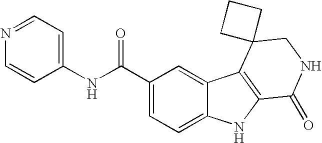 | 4,4-spirocyclobutyl-1-oxo-2,3,4,9- tetrahydro-1H-β-carboline-6- carboxylic acid pyridin-4-ylamide |
| 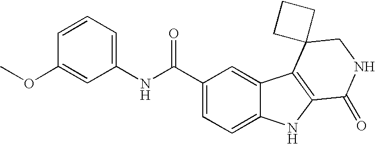 | 4,4-spirocyclobutyl-1-oxo-2,3,4,9- tetrahydro-1H-β-carboline-6- carboxylic acid (3-methoxy- phenyl)-amide |
| 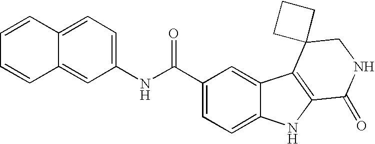 | 4,4-spirocyclobutyl-1-oxo-2,3,4,9- tetrahydro-1H-β-carboline-6- carboxylic acid naphthalen-2- ylamide |
| 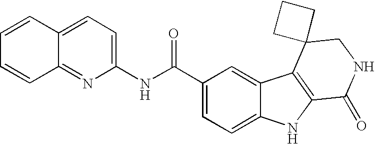 | 4,4-spirocyclobutyl-1-oxo-2,3,4,9- tetrahydro-1H-β-carboline-6- carboxylic acid quinolin-3-ylamide |
| 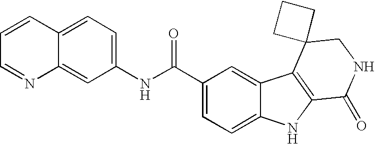 | 4,4-spirocyclobutyl-1-oxo-2,3,4,9- tetrahydro-1H-β-carboline-6- carboxylic acid quinolin-6-ylamide |
| 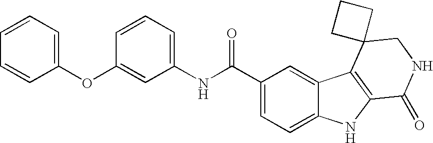 | 4,4-spirocyclobutyl-1-oxo-2,3,4,9- tetrahydro-1H-β-carboline-6- carboxylic acid (3-phenoxy- phenyl)-amide |
| 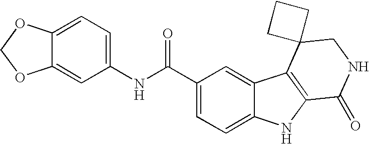 | 4,4-spirocyclobutyl-1-oxo-2,3,4,9- tetrahydro-1H-β-carboline-6- carboxylic acid benzo[1,3]dioxol- 5-ylamide |
| 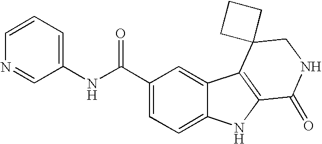 | 4,4-spirocyclobutyl-1-oxo-2,3,4,9- tetrahydro-1H-β-carboline-6- carboxylic acid pyridin-3-ylamide |
| 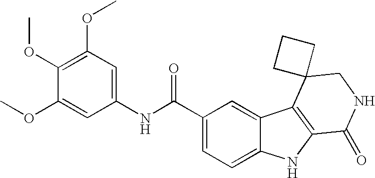 | 4,4-spirocyclobutyl-1-oxo-2,3,4,9- tetrahydro-1H-β-carboline-6- carboxylic acid (3,4,5-trimethoxy- phenyl)-amide |
| 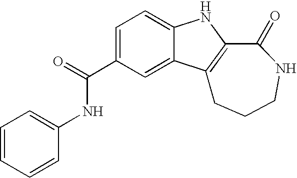 | 1-Oxo-1,2,3,4,5,10-hexahydro- azepino[3,4-β]indole-7-carboxylic acid phenylamide |
| 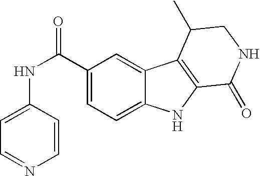 | 4-Methyl-1-oxo-2,3,4,9- tetrahydro-1H-β-carboline-6- carboxylic acid pyridin-4-ylamide |
| 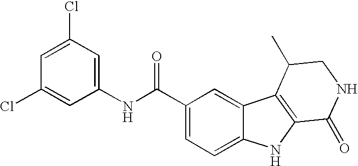 | 4-Methyl-1-oxo-2,3,4,9- tetrahydro-1H-β-carboline-6- carboxylic acid (3,5-dichloro- phenyl)-amide |
| 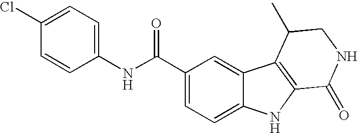 | 4-Methyl-1-oxo-2,3,4,9- tetrahydro-1H-β-carboline-6- carboxylic acid (4-chloro-phenyl)- amide |
| 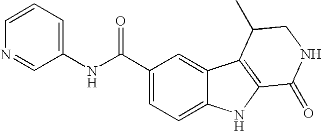 | 4-Methyl-1-oxo-2,3,4,9- tetrahydro-1H-β-carboline-6- carboxylic acid pyridin-3-ylamide |
| 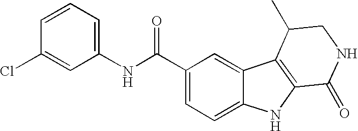 | 4-Methyl-1-oxo-2,3,4,9- tetrahydro-1H-β-carboline-6- carboxylic acid (3-chloro-phenyl)- amide |
| 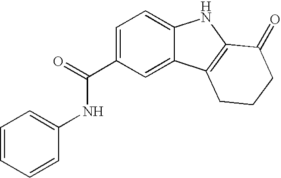 | 8-Oxo-6,7,8,9-tetrahydro-5H- carbazole-3-carboxylic acid phenylamide |
| 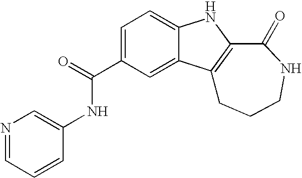 | 1-Oxo-1,2,3,4,5,10-hexahydro- azepino[3,4-b]indole-7-carboxylic acid pyridin-3-ylamide |
| 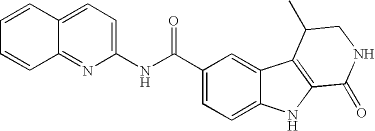 | 4-Methyl-1-oxo-2,3,4,9- tetrahydro-1H-β-carboline-6- carboxylic acid quinolin-3-ylamide |
| 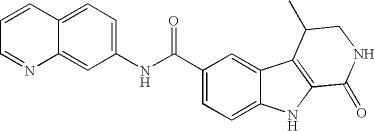 | 4-Methyl-1-oxo-2,3,4,9- tetrahydro-1H-β-carboline-6- carboxylic acid quinolin-6-ylamide |
| 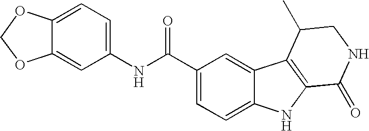 | 4-Methyl-1-oxo-2,3,4,9- tetrahydro-1H-b-carboline-6- carboxylic acid benzo[1,3]dioxol- 5-ylamide |
| 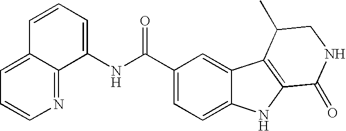 | 4-Methyl-1-oxo-2,3,4,9- tetrahydro-1H-b-carboline-6- carboxylic acid quinolin-8-ylamide |
| 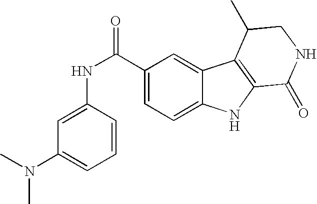 | 4-Methyl-1-oxo-2,3,4,9- tetrahydro-1H-β-carboline-6- carboxylic acid (3-dimethylamino- phenyl)-amide |
| 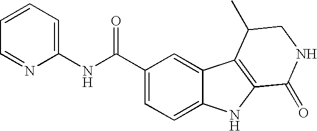 | 4-Methyl-1-oxo-2,3,4,9- tetrahydro-1H-β-carboline-6- carboxylic acid pyridin-2-ylamide |
| 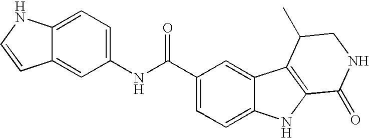 | 4-Methyl-1-oxo-2,3,4,9- tetrahydro-1H-β-carboline-6- carboxylic acid (1H-indol-6-yl)- amide |
| 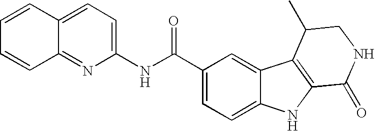 | 4-Methyl-1-oxo-2,3,4,9- tetrahydro-1H-β-carboline-6- carboxylic acid quinolin-2-ylamide |
| 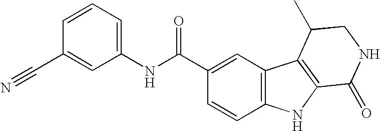 | 4-Methyl-1-oxo-2,3,4,9- tetrahydro-1H-β-carboline-6- carboxylic acid (3-cyano-phenyl)- amide |
| 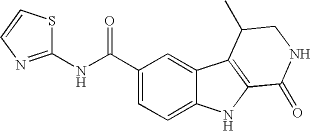 | 4-Methyl-1-oxo-2,3,4,9- tetrahydro-1H-β-carboline-6- carboxylic acid thiazol-2-ylamide |
| 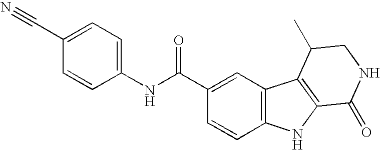 | 4-Methyl-1-oxo-2,3,4,9- tetrahydro-1H-β-carboline-6- carboxylic acid thiazol-2-ylamide |
| 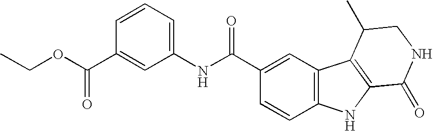 | 3-[(4-Methyl-1-oxo-2,3,4,9- tetrahydro-1H-β-carboline-6- carbonyl)-amino]-benzoic acid ethyl ester |
| 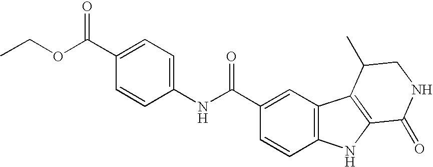 | 4-[(4-Methyl-1-oxo-2,3,4,9- tetrahydro-1H-β-carboline-6- carbonyl)-amino]-benzoic acid methyl ester |
| 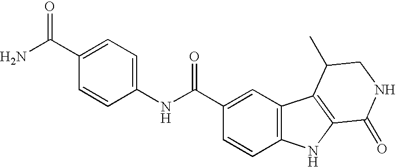 | 4-Methyl-1-oxo-2,3,4,9- tetrahydro-1H-β-carboline-6- carboxylic acid (4-carbamoyl- phenyl)-amide |
| 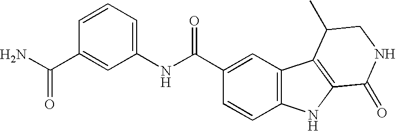 | 4-Methyl-1-oxo-2,3,4,9- tetrahydro-1H-β-carboline-6- carboxylic acid (3-carbamoyl- phenyl)-amide |
| 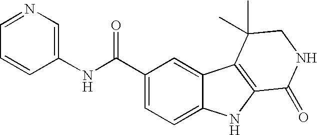 | 4,4-Dimethyl-1-oxo-2,3,4,9- tetrahydro-1H-β-carboline-6- carboxylic acid pyridin-3-ylamide |
| 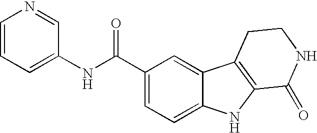 | 1-Oxo-2,3,4,9-tetrahydro-1H-β- carboline-6-carboxylic acid pyridin-3-ylamide |
| 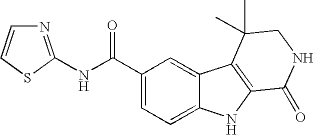 | 4,4-Dimethyl-1-oxo-2,3,4,9- tetrahydro-1H-β-carboline-6- carboxylic acid thiazol-2-ylamide |
| 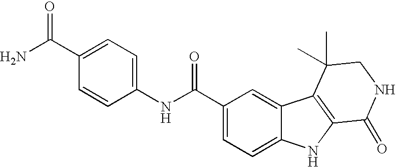 | 4,4-Dimethyl-1-oxo-2,3,4,9- tetrahydro-1H-β-carboline-6- carboxylic acid (4-carbamoyl- phenyl)-amide |
| 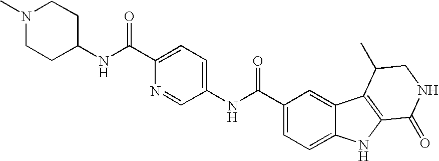 | 4-Methyl-1-oxo-2,3,4,9- tetrahydro-1H-β-carboline-6- carboxylic acid [6-(1-methyl- piperidin-4-ylcarbamoyl)-pyridin- 3-yl]-amide |
| 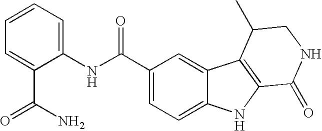 | 4-Methyl-1-oxo-2,3,4,9- tetrahydro-1H-β-carboline-6- carboxylic acid (2-carbamoyl- phenyl)-amide |
| 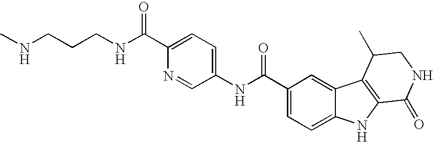 | 4-Methyl-1-oxo-2,3,4,9- tetrahydro-1H-β-carboline-6- carboxylic acid [6-(3- methylamino-propylcarbamoyl)- pyridin-3-yl]-amide |
| 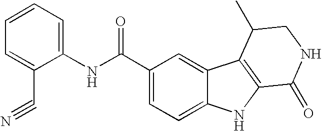 | 4-Methyl-1-oxo-2,3,4,9- tetrahydro-1H-β-carboline-6- carboxylic acid (2-cyano-phenyl)- amide |
| 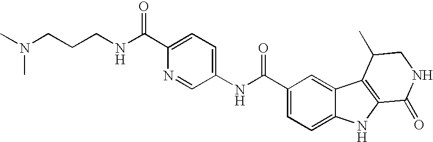 | 4-Methyl-1-oxo-2,3,4,9- tetrahydro-1H-β-carboline-6- carboxylic acid [6-(3- dimethylamino-propylcarbamoyl)- pyridin-3-yl]-amide |
| 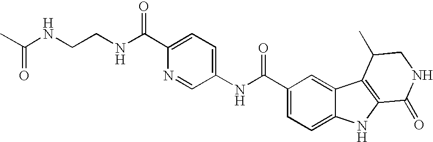 | 4-Methyl-1-oxo-2,3,4,9- tetrahydro-1H-β-carboline-6- carboxylic acid [6-(2-acetylamino- ethylcarbamoyl)-pyridin-3-yl]- amide |
| 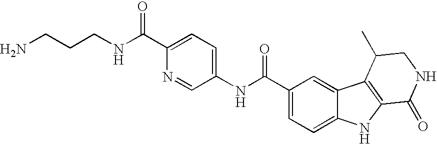 | 4-Methyl-1-oxo-2,3,4,9- tetrahydro-1H-β-carboline-6- carboxylic acid [6-(3-amino- propylcarbamoyl)-pyridin-3-yl]- amide |
| 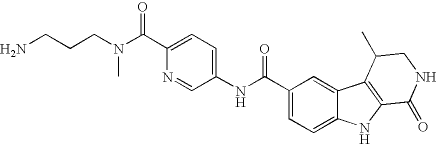 | 4-Methyl-1-oxo-2,3,4,9- tetrahydro-1H-β-carboline-6- carboxylic acid {6-[(3-amino- propyl)-methyl-carbamoyl]- pyridin-3-yl}-amide |
| 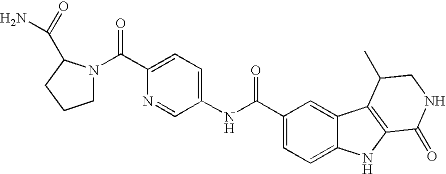 | 4-Methyl-1-oxo-2,3,4,9- tetrahydro-1H-β-carboline-6- carboxylic acid [6-(2-carbamoyl- pyrrolidine-1-carbonyl)-pyridin-3- yl]-amide |
| 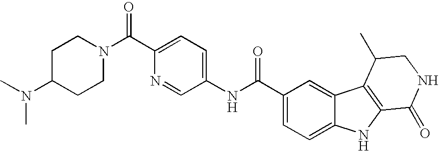 | 4-Methyl-1-oxo-2,3,4,9- tetrahydro-1H-β-carboline-6- carboxylic acid [6-(4- dimethylamino-piperidine-1- carbonyl)-pyridin-3-yl]-amide |
| 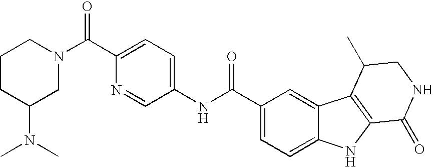 | 4-Methyl-1-oxo-2,3,4,9- tetrahydro-1H-β-carboline-6- carboxylic acid [6-(3- dimethylamino-piperidine-1- carbonyl)-pyridin-3-yl]-amide |
| 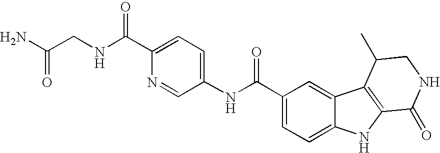 | 4-Methyl-1-oxo-2,3,4,9- tetrahydro-1H-β-carboline-6- carboxylic acid [6- (carbamoylmethyl-carbamoyl)- pyridin-3-yl]-amide |
| 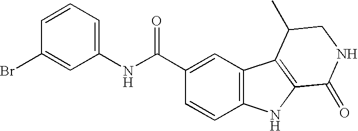 | 4-Methyl-1-oxo-2,3,4,9- tetrahydro-1H-β-carboline-6- carboxylic acid (3-bromo-phenyl)- amide |
| 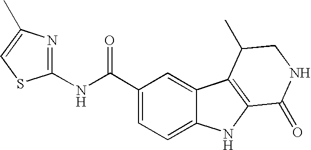 | 4-Methyl-1-oxo-2,3,4,9- tetrahydro-1H-β-carboline-6- carboxylic acid (4-methyl-thiazol- 2-yl)-amide |
| 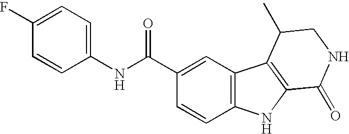 | 4-Methyl-1-oxo-2,3,4,9- tetrahydro-1H-β-carboline-6- carboxylic acid (4-fluoro-phenyl)- amide |
| 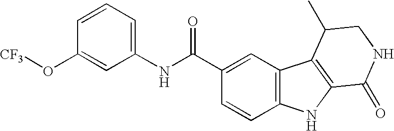 | 4-Methyl-1-oxo-2,3,4,9- tetrahydro-1H-β-carboline-6- carboxylic acid (3- trifluoromethoxy-phenyl)-amide |
| 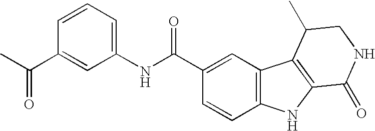 | 4-Methyl-1-oxo-2,3,4,9- tetrahydro-1H-β-carboline-6- carboxylic acid (3-acetyl-phenyl)- amide |
| 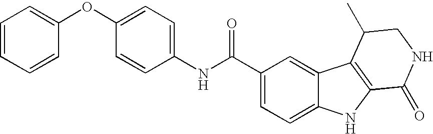 | 4-Methyl-1-oxo-2,3,4,9- tetrahydro-1H-β-carboline-6- carboxylic acid (4-phenoxy- phenyl)-amide |
| 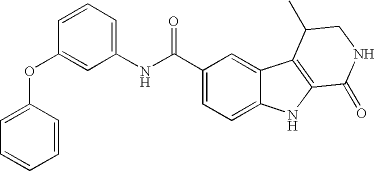 | 4-Methyl-1-oxo-2,3,4,9- tetrahydro-1H-β-carboline-6- carboxylic acid (3-phenoxy- phenyl)-amide |
| 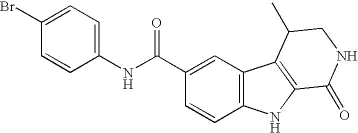 | 4-Methyl-1-oxo-2,3,4,9- tetrahydro-1H-β-carboline-6- carboxylic acid (4-bromo-phenyl)- amide |
| 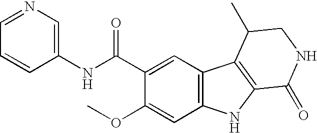 | 7-Methoxy-4-methyl-1-oxo 2,3,4,9-tetrahydro-1H-β- carboline-6-carboxylic acid pyridin-3-ylamide |
| 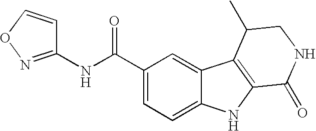 | 4-Methyl-1-oxo-2,3,4,9- tetrahydro-1H-β-carboline-6- carboxylic acid isoxazol-3- ylamide |
| 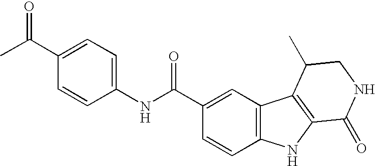 | 4-Methyl-1-oxo-2,3,4,9- tetrahydro-1H-β-carboline-6- carboxylic acid (4-acetyl-phenyl)- amide |
| 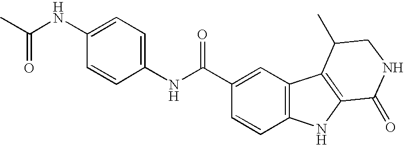 | 4-Methyl-1-oxo-2,3,4,9- tetrahydro-1H-β-carboline-6- carboxylic acid (4-acetylamino- phenyl)-amide |
| 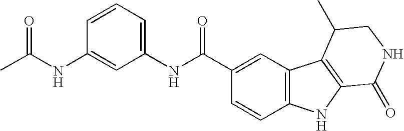 | 4-Methyl-1-oxo-2,3,4,9- tetrahydro-1H-β-carboline-6- carboxylic acid (3-acetylamino- phenyl)-amide |
| 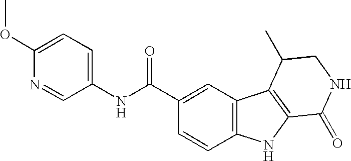 | 4-Methyl-1-oxo-2,3,4,9- tetrahydro-1H-β-carboline-6- carboxylic acid (6-methoxy- pyridin-3-yl)-amide |
| 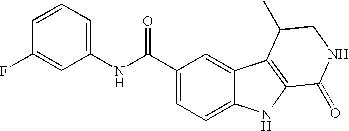 | 4-Methyl-1-oxo-2,3,4,9- tetrahydro-1H-β-carboline-6- carboxylic acid (3-fluoro-phenyl)- amide |
| 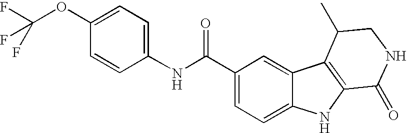 | 4-Methyl-1-oxo-2,3,4,9- tetrahydro-1H-β-carboline-6- carboxylic acid (4- trifluoromethoxy-phenyl)-amide |
| 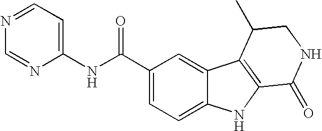 | 4-Methyl-1-oxo-2,3,4,9- tetrahydro-1H-β-carboline-6- carboxylic acid pyrimidin-4- ylamide |
| 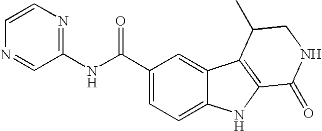 | 4-Methyl-1-oxo-2,3,4,9- tetrahydro-1H-β-carboline-6- carboxylic acid pyrazin-2-ylamide |
| 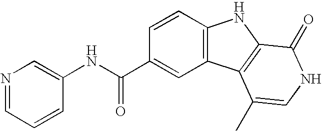 | 4-Methyl-1-oxo-2,9-dihydro-1H-β- carboline-6-carboxylic acid pyridin-3-ylamide |
| 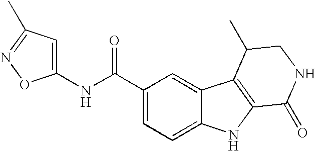 | 4-Methyl-1-oxo-2,3,4,9- tetrahydro-1H-β-carboline-6- carboxylic acid (3-methyl- isoxazol-5-yl)-amide |
| 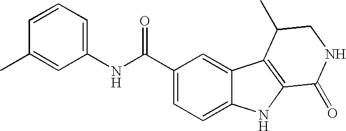 | 4-Methyl-1-oxo-2,3,4,9- tetrahydro-1H-β-carboline-6- carboxylic acid m-tolylamide |
| 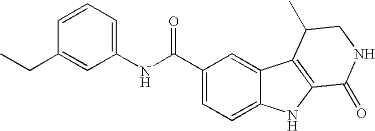 | 4-Methyl-1-oxo-2,3,4,9- tetrahydro-1H-β-carboline-6- carboxylic acid (3-ethyl-phenyl)- amide |
| 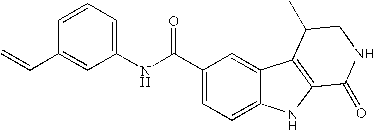 | 4-Methyl-1-oxo-2,3,4,9- tetrahydro-1H-β-carboline-6- carboxylic acid (3-vinyl-phenyl)- amide |
| 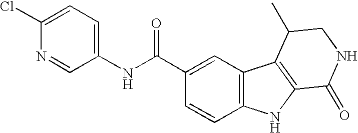 | 4-Methyl-1-oxo-2,3,4,9- tetrahydro-1H-β-carboline-6- carboxylic acid (6-chloro-pyridin- 3-yl)-amide |
| 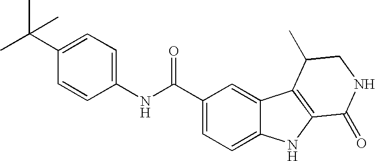 | 4-Methyl-1-oxo-2,3,4,9- tetrahydro-1H-β-carboline-6- carboxylic acid (4-tert-butyl- phenyl)-amide |
| 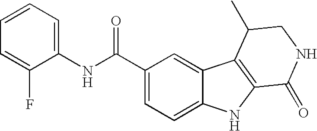 | 4-Methyl-1-oxo-2,3,4,9- tetrahydro-1H-β-carboline-6- carboxylic acid (2-fluoro-phenyl)- amide |
| 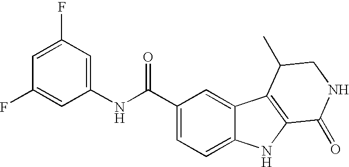 | 4-Methyl-1-oxo-2,3,4,9- tetrahydro-1H-β-carboline-6- carboxylic acid (3,5-difluoro- phenyl)-amide |
| 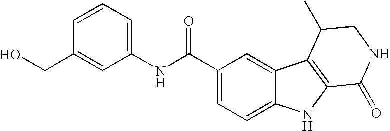 | 4-Methyl-1-oxo-2,3,4,9- tetrahydro-1H-β-carboline-6- carboxylic acid (3-hydroxymethyl- phenyl)-amide |
| 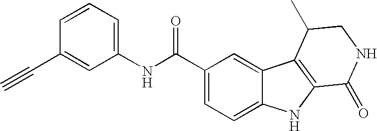 | 4-Methyl-1-oxo-2,3,4,9- tetrahydro-1H-β-carboline-6- carboxylic acid (3-ethynyl- phenyl)-amide |
| 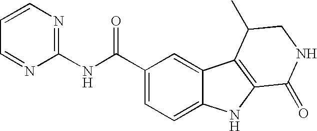 | 4-Methyl-1-oxo-2,3,4,9- tetrahydro-1H-β-carboline-6- carboxylic acid pyrimidin-2- ylamide |
| 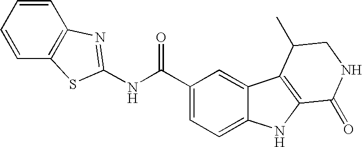 | 4-Methyl-1-oxo-2,3,4,9- tetrahydro-1H-β-carboline-6- carboxylic acid benzothiazol-2- ylamide |
| 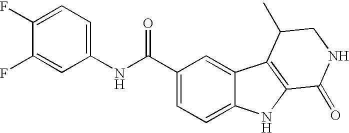 | 4-Methyl-1-oxo-2,3,4,9- tetrahydro-1H-β-carboline-6- carboxylic acid (3,4-difluoro- phenyl)-amide |
| 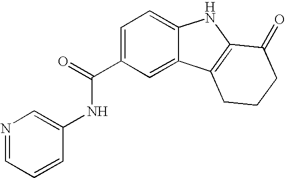 | 8-Oxo-6,7,8,9-tetrahydro-5H- carbazole-3-carboxylic acid pyridin-3-ylamide |
| 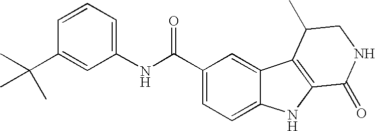 | 4-Methyl-1-oxo-2,3,4,9- tetrahydro-1H-β-carboline-6- carboxylic acid (3-tert-butyl- phenyl)-amide |
| 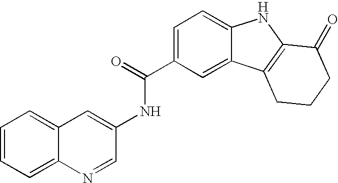 | 8-Oxo-6,7,8,9-tetrahydro-5H- carbazole-3-carboxylic acid quinolin-3-ylamide |
| 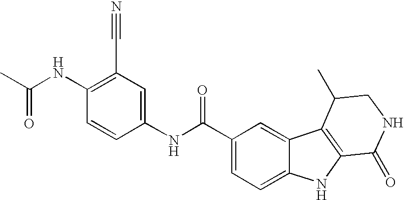 | 4-Methyl-1-oxo-2,3,4,9- tetrahydro-1H-β-carboline-6- carboxylic acid (4-acetylamino-3- cyano-phenyl)-amide |
| 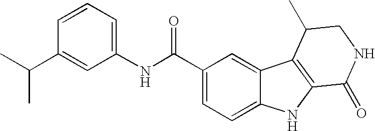 | 4-Methyl-1-oxo-2,3,4,9- tetrahydro-1H-β-carboline-6- carboxylic acid (3-isopropyl- phenyl)-amide |
| 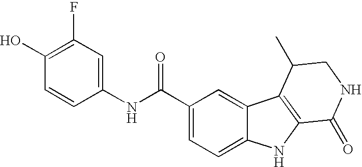 | 4-Methyl-1-oxo-2,3,4,9- tetrahydro-1H-β-carboline-6- carboxylic acid (3-fluoro-4- hydroxy-phenyl)-amide |
| 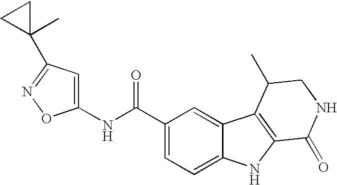 | 4-Methyl-1-oxo-2,3,4,9- tetrahydro-1H-β-carboline-6- carboxylic acid [3-(1-methyl- cyclopropyl)-isoxazol-5-yl]-amide |
| 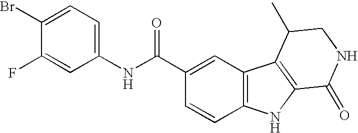 | 4-Methyl-1-oxo-2,3,4,9- tetrahydro-1H-β-carboline-6- carboxylic acid (4-bromo-3-fluoro- phenyl)-amide |
| 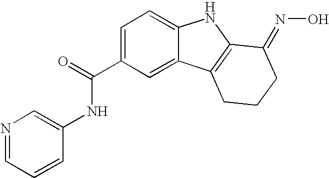 | 8-Hydroxyimino-6,7,8,9- tetrahydro-5H-carbazole-3- carboxylic acid pyridin-3-ylamide |
| 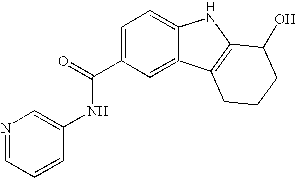 | 8-Hydroxy-6,7,8,9-tetrahydro-5H- carbazole-3-carboxylic acid pyridin-3-ylamide |
| 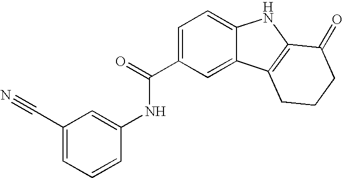 | 8-Oxo-6,7,8,9-tetrahydro-5H- carbazole-3-carboxylic acid (3- cyano-phenyl)-amide |
| 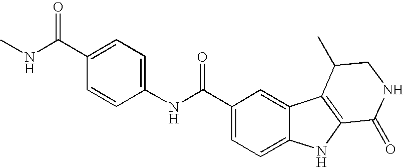 | 4-Methyl-1-oxo-2,3,4,9- tetrahydro-1H-β-carboline-6- carboxylic acid (4- methylcarbamoyl-phenyl)-amide |
| 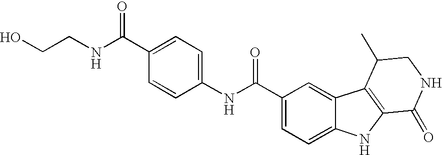 | 4-Methyl-1-oxo-2,3,4,9- tetrahydro-1H-β-carboline-6- carboxylic acid [4-(2-hydroxy- ethylcarbamoyl)-phenyl]-amide |
| 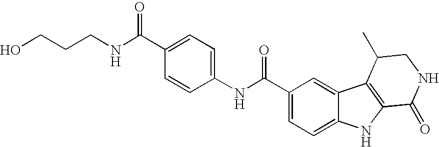 | 4-Methyl-1-oxo-2,3,4,9- tetrahydro-1H-β-carboline-6- carboxylic acid [4-(3-hydroxy- propylcarbamoyl)-phenyl]-amide |
| 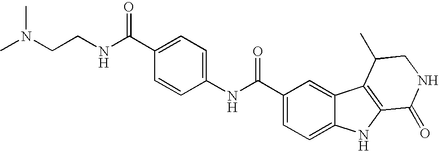 | 4-Methyl-1-oxo-2,3,4,9- tetrahydro-1H-β-carboline-6- carboxylic acid [4-(2- dimethylamino-ethylcarbamoyl)- phenyl]-amide |
| 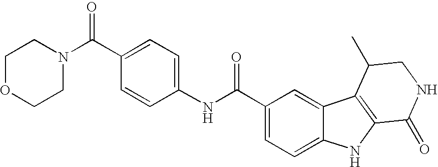 | 4-Methyl-1-oxo-2,3,4,9- tetrahydro-1H-β-carboline-6- carboxylic acid [4-(morpholine-4- carbonyl)-phenyl]-amide |
| 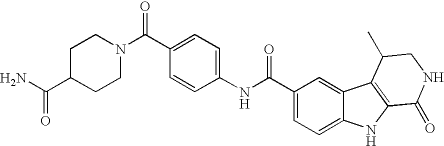 | 4-Methyl-1-oxo-2,3,4,9- tetrahydro-1H-β-carboline-6- carboxylic acid [4-(4-carbamoyl- piperidine-1-carbonyl)-phenyl]- amide |
| 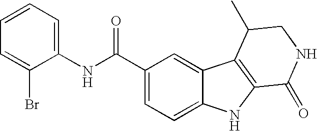 | 4-Methyl-1-oxo-2,3,4,9- tetrahydro-1H-β-carboline-6- carboxylic acid (2-bromo-phenyl)- amide |
| 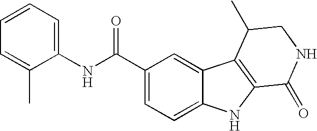 | 4-Methyl-1-oxo-2,3,4,9- tetrahydro-1H-β-carboline-6- carboxylic acid o-tolylamide |
| 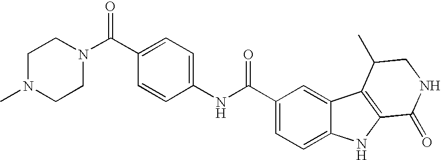 | 4-Methyl-1-oxo-2,3,4,9- tetrahydro-1H-β-carboline-6- carboxylic acid [4-(4-methyl- piperazine-1-carbonyl)-phenyl]- amide |
| 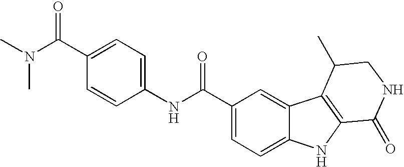 | 4-Methyl-1-oxo-2,3,4,9- tetrahydro-1H-β-carboline-6- carboxylic acid (4- dimethylcarbamoyl-phenyl)-amide |
| 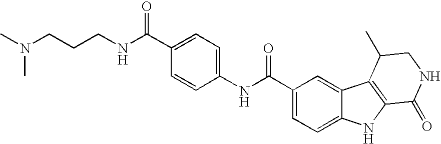 | 4-Methyl-1-oxo-2,3,4,9- tetrahydro-1H-β-carboline-6- carboxylic acid [4-(3- dimethylamino-propylcarbamoyl)- phenyl]-amide |
| 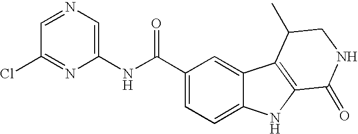 | 4-Methyl-1-oxo-2,3,4,9- tetrahydro-1H-β-carboline-6- carboxylic acid (6-chloro-pyrazin- 2-yl)-amide |
| 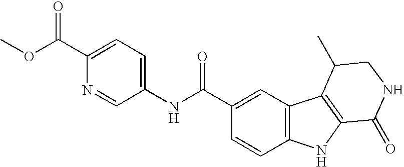 | 5-[(4-Methyl-1-oxo-2,3,4,9- tetrahydro-1H-β-carboline-6- carbonyl)-amino]-pyridine-2- carboxylic acid methyl ester |
| 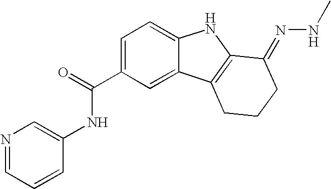 | 8-(Methyl-hydrazono)-6,7,8,9- tetrahydro-5H-carbazole-3- carboxylic acid pyridin-3-ylamide |
| 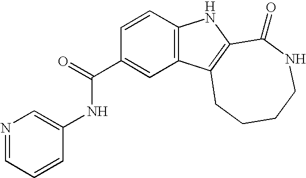 | 1-Oxo-2,3,4,5,6,11-hexahydro- 1H-azocino[3,4-b]indole-8- carboxylic acid pyridin-3-ylamide |
| 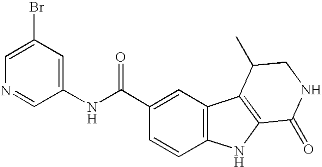 | 4-Methyl-1-oxo-2,3,4,9- tetrahydro-1H-β-carboline-6- carboxylic acid (5-bromo-pyridin- 3-yl)-amide |
| 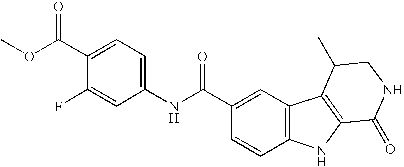 | 2-Fluoro-4-[(4-methyl-1-oxo- 2,3,4,9-tetrahydro-1H- β-carboline-6-carbonyl)-amino]- benzoic acid methyl ester |
| 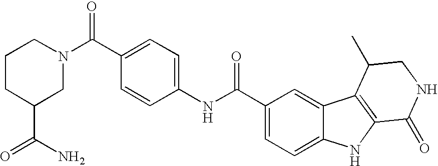 | 4-Methyl-1-oxo-2,3,4,9- tetrahydro-1H-β-carboline-6- carboxylic acid [4-(3-carbamoyl- piperidine-1-carbonyl)-phenyl]- amide |
| 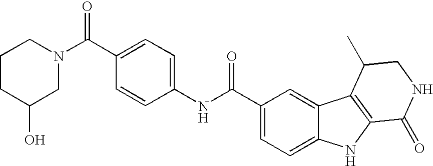 | 4-Methyl-1-oxo-2,3,4,9- tetrahydro-1H-β-carboline-6- carboxylic acid [4-(3-hydroxy- piperidine-1-carbonyl)-phenyl]- amide |
| 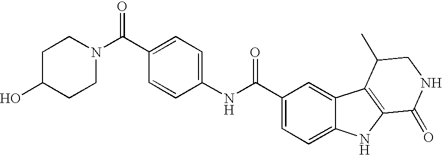 | 4-Methyl-1-oxo-2,3,4,9- tetrahydro-1H-β-carboline-6- carboxylic acid [4-(4-hydroxy- piperidine-1-carbonyl)-phenyl]- amide |
| 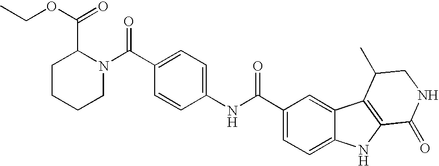 | 1-{4-[(4-Methyl-1-oxo-2,3,4,9- tetrahydro-1H-β-carboline-6- carbonyl)-amino]-benzoyl}- piperidine-2-carboxylic acid ethyl ester |
| 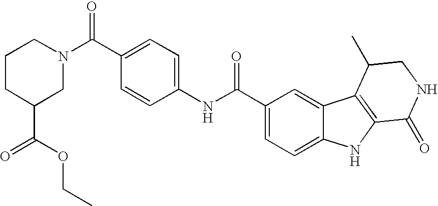 | 1-{4-[(4-Methyl-1-oxo-2,3,4,9- tetrahydro-1H-β-carboline-6- carbonyl)-amino]-benzoyl}- piperidine-3-carboxylic acid ethyl ester |
| 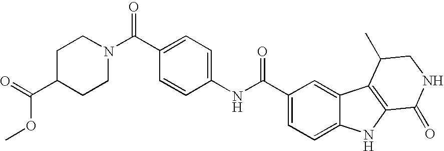 | 1 -{4-[(4-Methyl-1-oxo-2,3,4,9- tetrahydro-1H-β-carboline-6- carbonyl)-amino]-benzoyl}- piperidine-4-carboxylic acid methyl ester |
| 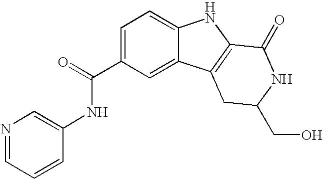 | 3-Hydroxymethyl-1-oxo-2,3,4,9- tetrahydro-1H-β-carboline-6- carboxylic acid pyridin-3-ylamide |
| 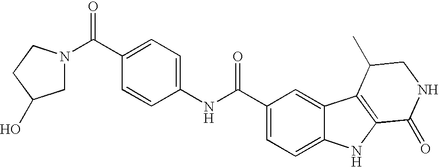 | 4-Methyl-1-oxo-2,3,4,9- tetrahydro-1H-β-carboline-6- carboxylic acid [4-(3-hydroxy- pyrrolidine-1-carbonyl)-phenyl]- amide |
| 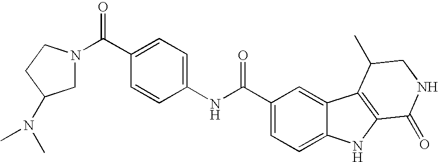 | 4-Methyl-1-oxo-2,3,4,9- tetrahydro-1H-β-carboline-6- carboxylic acid [4-(3- dimethylamino-pyrrolidine-1- carbonyl)-phenyl]-amide |
| 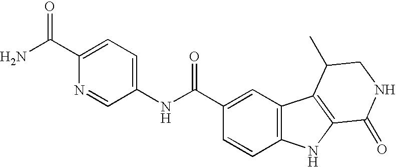 | 4-Methyl-1-oxo-2,3,4,9- tetrahydro-1H-β-carboline-6- carboxylic acid (6-carbamoyl- pyridin-3-yl)-amide |
| 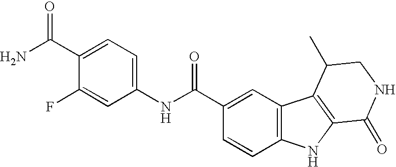 | 4-Methyl-1-oxo-2,3,4,9- tetrahydro-1H-β-carboline-6- carboxylic acid (4-carbamoyl-3- fluoro-phenyl)-amide |
| 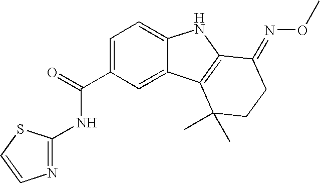 | 8-Methoxyimino-5,5-dimethyl- 6,7,8,9-tetrahydro-5H-carbazole- 3-carboxylic acid thiazol-2- ylamide |
| 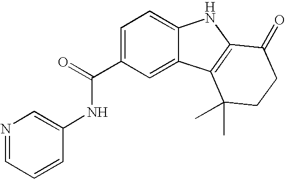 | 5,5-Dimethyl-8-oxo-6,7,8,9- tetrahydro-5H-carbazole-3- carboxylic acid pyridin-3-ylamide |
| 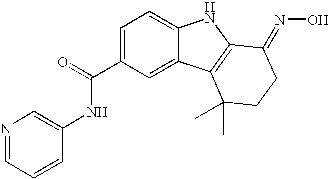 | 8-Hydroxyimino-5,5-dimethyl- 6,7,8,9-tetrahydro-5H-carbazole- 3-carboxylic acid pyridin-3- ylamide |
| 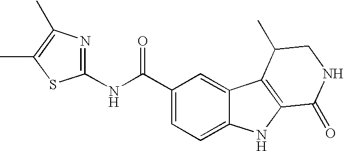 | 4-Methyl-1-oxo-2,3,4,9- tetrahydro-1H-β-carboline-6- carboxylic acid (4,5-dimethyl- thiazol-2-yl)-amide |
| 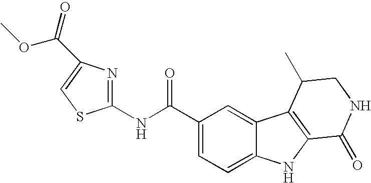 | 2-[(4-Methyl-1-oxo-2,3,4,9- tetrahydro-1H-β-carboline-6- carbonyl)-amino]-thiazole-4- carboxylic acid methyl ester |
| 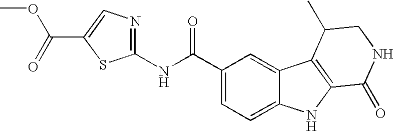 | 2-[(4-Methyl-1-oxo-2,3,4,9- tetrahydro-1H-β-carboline-6- carbonyl)-amino]-thiazole-5- carboxylic acid methyl ester |
| 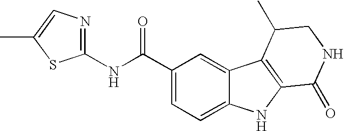 | 4-Methyl-1-oxo-2,3,4,9- tetrahydro-1H-β-carboline-6- carboxylic acid (5-methyl-thiazol- 2-yl)-amide |
| 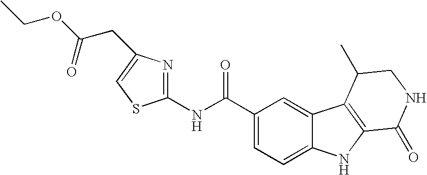 | {2-[(4-Methyl-1-oxo-2,3,4,9- tetrahydro-1H-β-carboline-6- carbonyl)-amino]-thiazol-4-yl}- acetic acid ethyl ester |
| 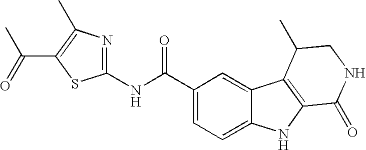 | 4-Methyl-1-oxo-2,3,4,9- tetrahydro-1H-β-carboline-6- carboxylic acid (5-acetyl-4- methyl-thiazol-2-yl)-amide |
| 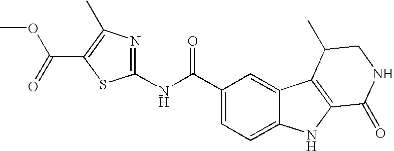 | 4-Methyl-2-[(4-methyl-1-oxo- 2,3,4,9-tetrahydro-1H- β-carboline-6-carbonyl)-amino]- thiazole-5-carboxylic acid methyl ester |
| 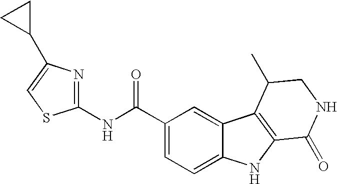 | 4-Methyl-1-oxo-2,3,4,9- tetrahydro-1H-β-carboline-6- carboxylic acid (4-cyclopropyl- thiazol-2-yl)-amide |
| 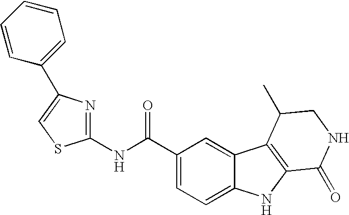 | 4-Methyl-1-oxo-2,3,4,9- tetrahydro-1H-β-carboline-6- carboxylic acid (4-phenyl-thiazol- 2-yl)-amide |
| 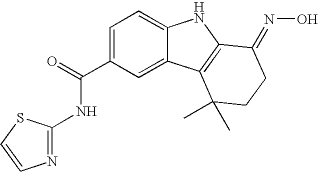 | 8-Hydroxyimino-5,5-dimethyl- 6,7,8,9-tetrahydro-5H-carbazole- 3-carboxylic acid thiazol-2- ylamide |
| 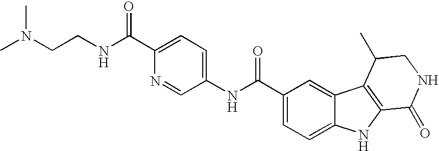 | 4-Methyl-1-oxo-2,3,4,9- tetrahydro-1H-β-carboline-6- carboxylic acid [6-(2- dimethylamino-ethylcarbamoyl)- pyridin-3-yl]-amide |
| 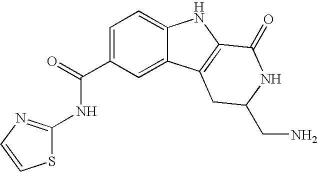 | 3-Aminomethyl-1-oxo-2,3,4,9- tetrahydro-1H-β-carboline-6- carboxylic acid thiazol-2-ylamide |
| 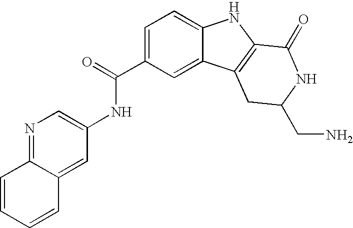 | 3-Aminomethyl-1-oxo-2,3,4,9- tetrahydro-1H-β-carboline-6- carboxylic acid quinolin-3-ylamide |
| 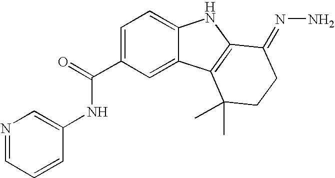 | 8-Hydrazono-5,5-dimethyl- 6,7,8,9-tetrahydro-5H-carbazole- 3-carboxylic acid pyridin-3- ylamide |
| 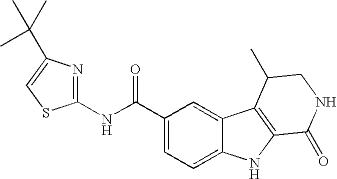 | 4-Methyl-1-oxo-2,3,4,9- tetrahydro-1H-β-carboline-6- carboxylic acid (4-tert-butyl- thiazol-2-yl)-amide |
| 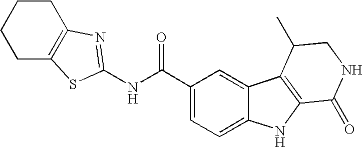 | 4-Methyl-1-oxo-2,3,4,9- tetrahydro-1H-β-carboline-6- carboxylic acid (4,5,6,7- tetrahydro-benzothiazol-2-yl)- amide |
| 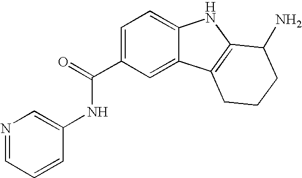 | 8-Amino-6,7,8,9-tetrahydro-5H- carbazole-3-carboxylic acid pyridin-3-ylamide |
| 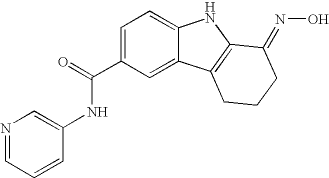 | 8-Hydroxyimino-6,7,8,9- tetrahydro-5H-carbazole-3- carboxylic acid pyridin-3-ylamide |
| 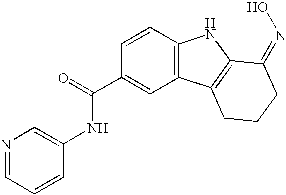 | 8-[(Z)-Hydroxyimino]-6,7,8,9- tetrahydro-5H-carbazole-3- carboxylic acid pyridin-3-ylamide |
| 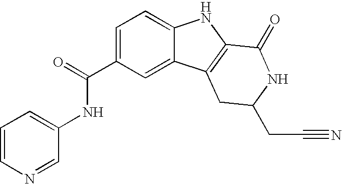 | 3-Cyanomethyl-1-oxo-2,3,4,9- tetrahydro-1H-β-carboline-6- carboxylic acid pyridin-3-ylamide |
| 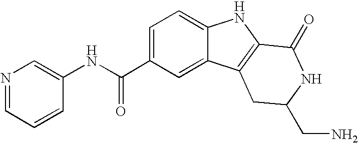 | 3-Aminomethyl-1-oxo-2,3,4,9- tetrahydro-1H-β-carboline-6- carboxylic acid pyridin-3-ylamide |
| 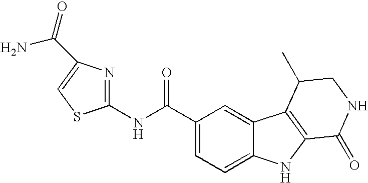 | 4-Methyl-1-oxo-2,3,4,9- tetrahydro-1H-β-carboline-6- carboxylic acid (4-carbamoyl- thiazol-2-yl)-amide |
| 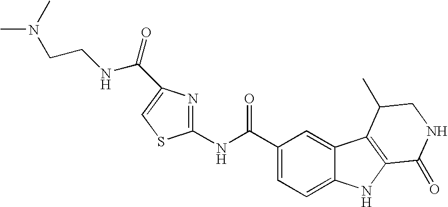 | 4-Methyl-1-oxo-2,3,4,9- tetrahydro-1H-β-carboline-6- carboxylic acid [4-(2- dimethylamino-ethylcarbamoyl)- thiazol-2-yl]-amide |
| 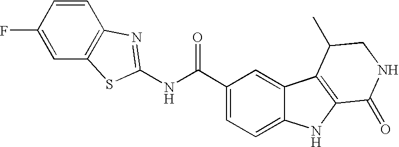 | 4-Methyl-1-oxo-2,3,4,9- tetrahydro-1H-β-carboline-6- carboxylic acid (6-fluoro- benzothiazol-2-yl)-amide |
| 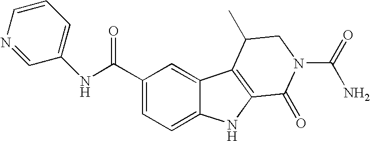 | 4-Methyl-1-oxo-1,3,4,9- tetrahydro-β-carboline-2,6- dicarboxylic acid 2-amide 6- pyridin-3-ylamide |
| 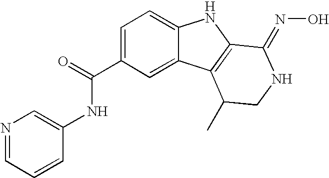 | 1-[(Z)-Hydroxyimino]-4-methyl- 2,3,4,9-tetrahydro-1H-β- carboline-6-carboxylic acid pyridin-3-ylamide |
| 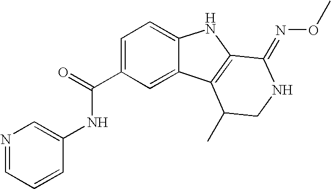 | 1-[(Z)-Methoxyimino]-4-methyl- 2,3,4,9-tetrahydro-1H-β- carboline-6-carboxylic acid pyridin-3-ylamide |
| 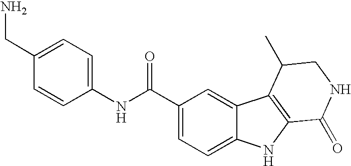 | 4-Methyl-1-oxo-2,3,4,9- tetrahydro-1H-β-carboline-6- carboxylic acid (4-aminomethyl- phenyl)-amide |
| 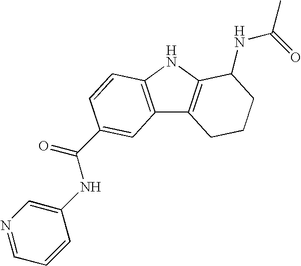 | 8-Acetylamino-6,7,8,9-tetrahydro- 5H-carbazole-3-carboxylic acid pyridin-3-ylamide |
| 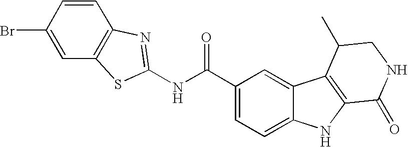 | 4-Methyl-1-oxo-2,3,4,9- tetrahydro-1H-β-carboline-6- carboxylic acid (6-bromo- benzothiazol-2-yl)-amide |
| 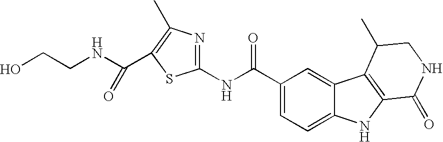 | 4-Methyl-1-oxo-2,3,4,9- tetrahydro-1H-β-carboline-6- carboxylic acid [5-(2-hydroxy- ethylcarbamoyl)-4-methyl-thiazol- 2-yl]-amide |
| 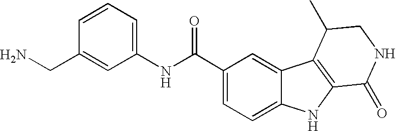 | 4-Methyl-1-oxo-2,3,4,9- tetrahydro-1H-β-carboline-6- carboxylic acid (3-aminomethyl- phenyl)-amide |
| 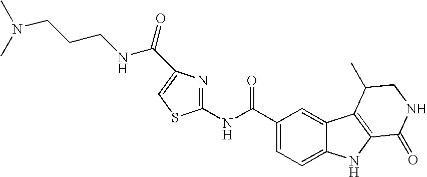 | 4-Methyl-1-oxo-2,3,4,9- tetrahydro-1H-β-carboline-6- carboxylic acid [4-(3- dimethylamino-propylcarbamoyl)- thiazol-2-yl]-amide |
| 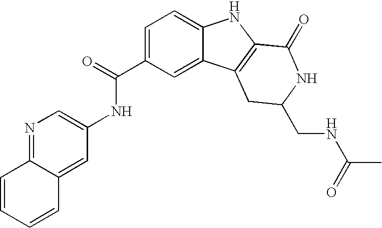 | 3-(Acetylamino-methyl)-1-oxo- 2,3,4,9-tetrahydro-1H-b- carboline-6-carboxylic acid quinolin-3-ylamide |
| 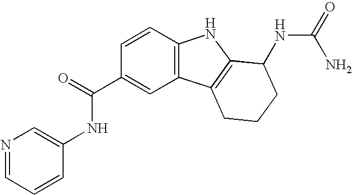 | 8-Ureido-6,7,8,9-tetrahydro-5H- carbazole-3-carboxylic acid pyridin-3-ylamide |
| 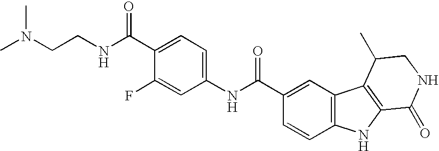 | 4-Methyl-1-oxo-2,3,4,9- tetrahydro-1H-β-carboline-6- carboxylic acid [4-(2- dimethylamino-ethylcarbamoyl)- 3-fluoro-phenyl]-amide |
| 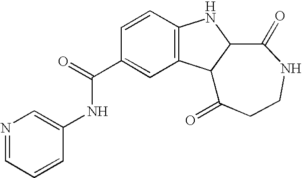 | 1,5-Dioxo-1,2,3,4,5,10- hexahydro-azepino[3,4-β]indole- 7-carboxylic acid pyridin-3- ylamide |
| 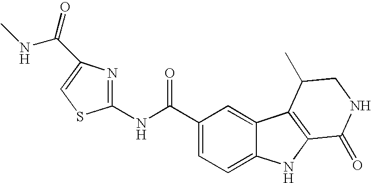 | 4-Methyl-1-oxo-2,3,4,9- tetrahydro-1H-β-carboline-6- carboxylic acid (4- methylcarbamoyl-thiazol-2-yl)- amide |
| 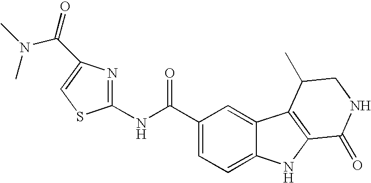 | 4-Methyl-1-oxo-2,3,4,9- tetrahydro-1H-β-carboline-6- carboxylic acid (4- dimethylcarbamoyl-thiazol-2-yl)- amide |
| 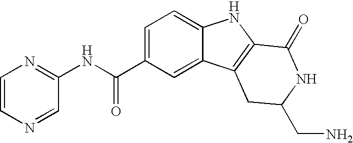 | 3-Aminomethyl-1-oxo-2,3,4,9- tetrahydro-1H-β-carboline-6- carboxylic acid pyrazin-2-ylamide |
| 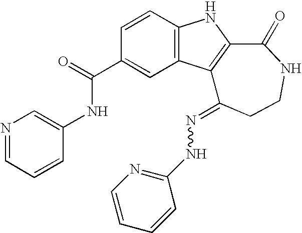 | 1-Oxo-5-(pyridin-2-yl-hydrazono)- 1,2,3,4,5,10-hexahydro- azepino[3,4-β]indole-7-carboxylic acid pyridin-3-ylamide |
| 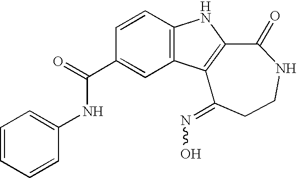 | 5-Hydroxyimino-1-oxo- 1,2,3,4,5,10-hexahydro- azepino[3,4-β]indole-7-carboxylic acid phenylamide |
| 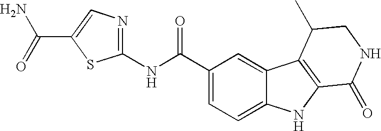 | 4-Methyl-1-oxo-2,3,4,9- tetrahydro-1H-β-carboline-6- carboxylic acid (5-carbamoyl- thiazol-2-yl)-amide |
| 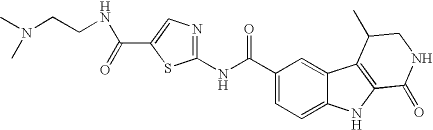 | 4-Methyl-1-oxo-2,3,4,9- tetrahydro-1H-β-carboline-6- carboxylic acid [5-(2- dimethylamino-ethylcarbamoyl)- thiazol-2-yl]-amide |
| 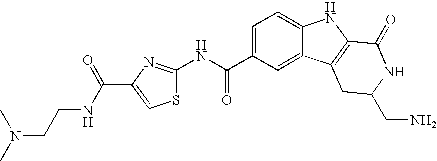 | 3-Aminomethyl-1-oxo-2,3,4,9- tetrahydro-1H-β-carboline-6- carboxylic acid [4-(2- dimethylamino-ethylcarbamoyl)- thiazol-2-yl]-amide |
| 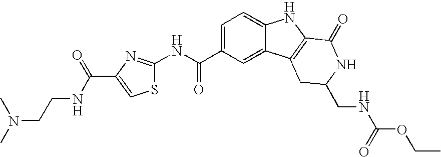 | {6-[4-(2-Dimethylamino- ethylcarbamoyl)-thiazol-2- ylcarbamoyl]-1-oxo-2,3,4,9- tetrahydro-1H-β-carbolin-3- ylmethyl}-carbamic acid ethyl ester |
| 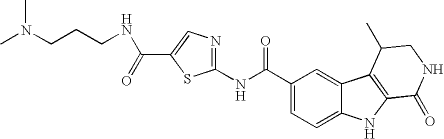 | 4-Methyl-1-oxo-2,3,4,9- tetrahydro-1H-β-carboline-6- carboxylic acid [5-(3- dimethylamino-propylcarbamoyl)- thiazol-2-yl]-amide |

In still another embodiment, there are provided the following preferred compounds that according to the present invention having an IC₅₀≦500 nM are provided in Table 2 hereinbelow.

TABLE 2
Preferred Compound Structures and Names

| | |
|---|---|
| 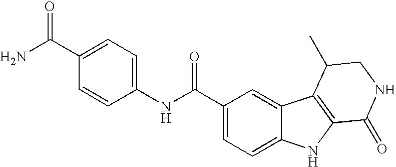 | 4-Methyl-1-oxo-2,3,4,9-tetrahydro- 1H-β-carboline-6-carboxylic acid (4- carbamoyl-phenyl)-amide |
| 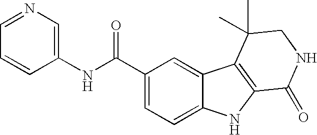 | 4,4-Dimethyl-1-oxo-2,3,4,9- tetrahydro-1H-β-carboline-6- carboxylic acid pyridin-3-ylamide |
| 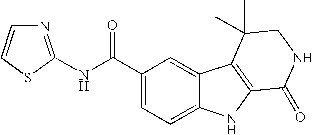 | 4,4-Dimethyl-1-oxo-2,3,4,9- tetrahydro-1H-β-carboline-6- carboxylic acid thiazol-2-ylamide |
| 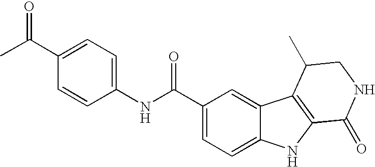 | 4-Methyl-1-oxo-2,3,4,9-tetrahydro- 1H-β-carboline-6-carboxylic acid (4- acetyl-phenyl)-amide |
| 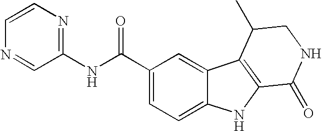 | 4-Methyl-1-oxo-2,3,4,9-tetrahydro- 1H-β-carboline-6-carboxylic acid pyrazin-2-ylamide |
| 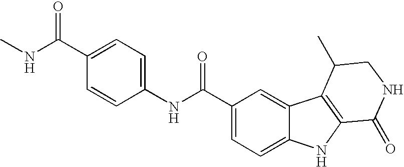 | 4-Methyl-1-oxo-2,3,4,9-tetrahydro- 1H-β-carboline-6-carboxylic acid (4- methylcarbamoyl-phenyl)-amide |
| 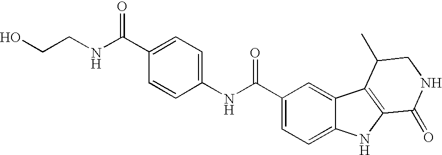 | 4-Methyl-1-oxo-2,3,4,9-tetrahydro- 1H-β-carboline-6-carboxylic acid [4- (2-hydroxy-ethylcarbamoyl)-phenyl]- amide |
| 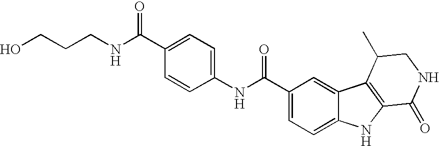 | 4-Methyl-1-oxo-2,3,4,9-tetrahydro- 1H-β-carboline-6-carboxylic acid [4- (3-hydroxy-propylcarbamoyl)- phenyl]-amide |
| 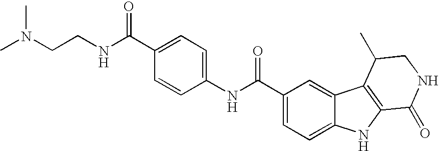 | 4-Methyl-1-oxo-2,3,4,9-tetrahydro- 1H-β-carboline-6-carboxylic acid [4- (2-dimethylamino-ethylcarbamoyl)- phenyl]-amide |
| 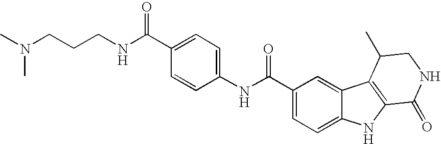 | 4-Methyl-1-oxo-2,3,4,9-tetrahydro- 1H-β-carboline-6-carboxylic acid [4- (3-dimethylamino-propylcarbamoyl)- phenyl]-amide |
| 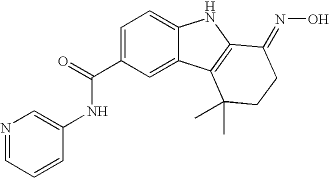 | 8-Hydroxyimino-5,5-dimethyl- 6,7,8,9-tetrahydro-5H-carbazole-3- carboxylic acid pyridin-3-ylamide |
| 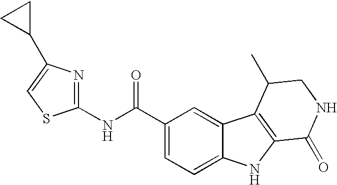 | 4-Methyl-1-oxo-2,3,4,9-tetrahydro- 1H-β-carboline-6-carboxylic acid (4- cyclopropyl-thiazol-2-yl)-amide |
| 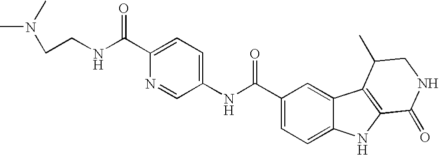 | 4-Methyl-1-oxo-2,3,4,9-tetrahydro- 1H-β-carboline-6-carboxylic acid [6- (2-dimethylamino-ethylcarbamoyl)- pyridin-3-yl]-amide |
| 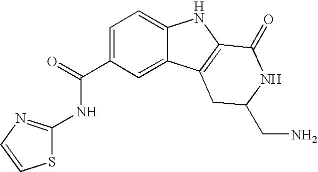 | 3-Aminomethyl-1-oxo-2,3,4,9- tetrahydro-1H-β-carboline-6- carboxylic acid thiazol-2-ylamide |
| 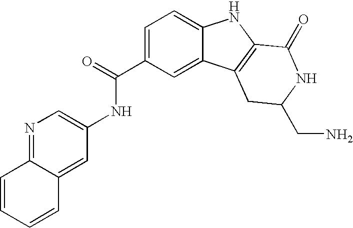 | 3-Aminomethyl-1-oxo-2,3,4,9- tetrahydro-1H-β-carboline-6- carboxylic acid quinolin-3-ylamide |
| 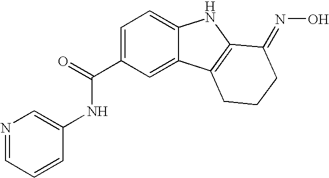 | 8-Hydroxyimino-6,7,8,9-tetrahydro- 5H-carbazole-3-carboxylic acid pyridin-3-ylamide |
| 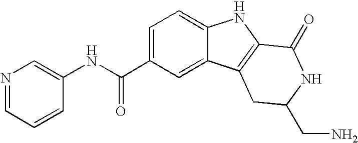 | 3-Aminomethyl-1-oxo-2,3,4,9- tetrahydro-1H-β-carboline-6- carboxylic acid pyridin-3-ylamide |
| 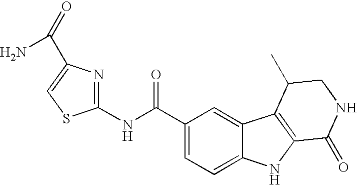 | 4-Methyl-1-oxo-2,3,4,9-tetrahydro- 1H-β-carboline-6-carboxylic acid (4- carbamoyl-thiazol-2-yl)-amide |
| 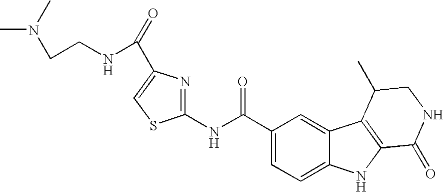 | 4-Methyl-1-oxo-2,3,4,9-tetrahydro- 1H-β-carboline-6-carboxylic acid [4- (2-dimethylamino-ethylcarbamoyl)- thiazol-2-yl]-amide |
| 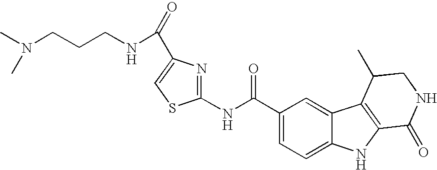 | 4-Methyl-1-oxo-2,3,4,9-tetrahydro- 1H-β-carboline-6-carboxylic acid [4- (3-dimethylamino-propylcarbamoyl)- thiazol-2-yl]-amide |

In all the compounds disclosed in this application, in the event the nomenclature is in conflict with the structure, it shall be understood that the compound is defined by the structure.

The invention includes the use of any compounds that contain one or more asymmetric carbon atoms which may occur as racemates and racemic mixtures, single enantiomers, diastereomeric mixtures and individual diastereomers. All such isomeric forms of these compounds are expressly included in the present invention. Each stereogenic carbon may be in the R or S configuration, or a combination of configurations.

Some of the compounds of formula (I) can exist in more than one tautomeric form. The invention includes methods using all such tautomers.

All terms as used herein in this specification, unless otherwise stated, shall be understood in their ordinary meaning as known in the art. For example, “C₁₋₆ alkoxy” is a C₁₋₄alkyl that contain an oxygen atom, such as methoxy, ethoxy, propoxy, butoxy. All alkyl, alkenyl and alkynyl groups shall be understood as being branched or unbranched where structurally possible and unless otherwise specified. Other more specific definitions are as follows:

The term “cycloalkyl” shall be understood to mean an aliphatic hydrocarbon radical containing from three to twelve carbon atoms. Cycloalkyls include hydrocarbon rings containing from three to ten carbon atoms. These cycloalkyls may be either aromatic and non-aromatic ring systems. The non-aromatic ring systems may be mono- or polyunsaturated. Preferred cycloalkyls include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used inerchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure. Unless otherwise stated, heterocycles include but are not limited to, for example pyrrolidinyl, pyrrolinyl, morpholinyl, thiomorpholinyl, thiomorpholinyl sulfoxide, thiomorpholinyl sulfone, dioxalanyl, piperidinyl, piperazinyl, tetrahydrofuranyl, 1-oxo-λ4-thiomorpholinyl, 13-oxa-11-aza-tricyclo[7.3.1.0-2,7]trideca-2,4,6-triene, tetrahydropyranyl, 2-oxo-2H-pyranyl, tetrahydrofuranyl, 1,3-dioxolanone, 1,3-dioxanone, 1,4-dioxanyl, 8-oxa-3-aza-bicyclo[3.2.1]octanyl, 2-oxa-5-aza-bicyclo[2.2.1]heptanyl, 2-thia-5-aza-bicyclo[2.2.1]heptanyl, piperidinonyl, tetrahydropyrimidonyl, pentamethylene sulfide, pentamethylene sulfoxide, pentamethylene sulfone, tetramethylene sulfide, tetramethylene sulfoxide and tetramethylene sulfone.

The term “heteroaryl” shall be understood to mean an aromatic 5-8 membered monocyclic or 8-11 membered bicyclic ring containing 1-4 heteroatoms such as N, O and S. Unless otherwise stated, such heteroaryls include aziridinyl, thienyl, furanyl, isoxazolyl, oxazolyl, thiazolyl, thiadiazolyl, tetrazolyl, pyrazolyl, pyrrolyl, imidazolyl, pyridinyl, pyrimidinyl, pyrazinyl, pyridazinyl, pyranyl, quinoxalinyl, indolyl, benzimidazolyl, benzoxazolyl, benzothiazolyl, benzothienyl, quinolinyl, quinazolinyl, naphthyridinyl, indazolyl, triazolyl, pyrazolo[3,4-b]pyrimidinyl, purinyl, pyrrolo[2,3-b]pyridinyl, pyrazolo[3,4-b]pyridinyl, tubercidinyl, oxazo[4,5-b]pyridinyl and imidazo[4,5-b]pyridinyl.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in defintions such as but not limited to: alkoxycarbonyl, acyl, amido and thioxo.

The term “aryl” as used herein shall be understood to mean aromatic cycloalkyl or heteroaryl as defined herein. Each aryl or heteroaryl unless otherwise specified includes it's partially or fully hydrogenated derivative. For example, quinolinyl may include decahydroquinolinyl and tetrahydroquinolinyl, naphthyl may include it's hydrogenated derivatives such as tetrahydranaphthyl. Other partially or fully hydrogenated derivatives of the aryl and heteroaryl compounds described herein will be apparent to one of ordinary skill in the art.

Terms which are analogs of the above cyclic moieties such as aryloxy or heteroaryl amine shall be understood to mean an aryl, heteroaryl, heterocycle as defined above attached to it's respective group.

As used herein, “nitrogen” and “sulfur” include any oxidized form of nitrogen and sulfur and the quaternized form of any basic nitrogen. For example, for an —S—C₁₋₆ alkyl radical, unless otherwise specified, this shall be understood to include —S(O)—C₁₋₆ alkyl and —S(O)₂—C₁₋₆ alkyl.

The term “halogen” as used in the present specification shall be understood to mean bromine, chlorine, fluorine or iodine. The definitions “partially or fully halogenated” “substituted by one or more halogen atoms” includes for example, mono, di or tri halo derivatives on one or more carbon atoms. For alkyl, a nonlimiting example would be —CH₂CHF₂, —CF₃ etc.

The term “ureido”, the present specification, is having the general formula of either C(O)NR^{x}R^{y}, NHC(O)R^{x}.

The term “carbamoyl”, the present specification, is substituent having the general formula C(O)NR^{x}R^{y} or NHC(O)R^{x}.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C1-C4 alkyl)4+ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

General Synthetic Methods

The invention additionally provides for methods of making the compounds of formula (I). The compounds of the invention may be prepared by the general methods and examples presented below, and methods known to those of ordinary skill in the art. Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. In the schemes below, unless otherwise specified, X and R¹-R¹³ in the formulas shown below shall have the meanings defined for these groups in the definition of the formula (I) of the invention, described hereinabove. Intermediates used in the syntheses below are either commercially available or easily prepared by methods known to those skilled in the art. Reaction progress may be monitored by conventional methods such as thin layer chromatography (TLC). Intermediates and products may be purified by methods known in the art, including column chromatography, HPLC or recrystallization.

Compounds of formula I having X═C, R¹²=oxo and R¹³ not present may be prepared as illustrated in Scheme I.

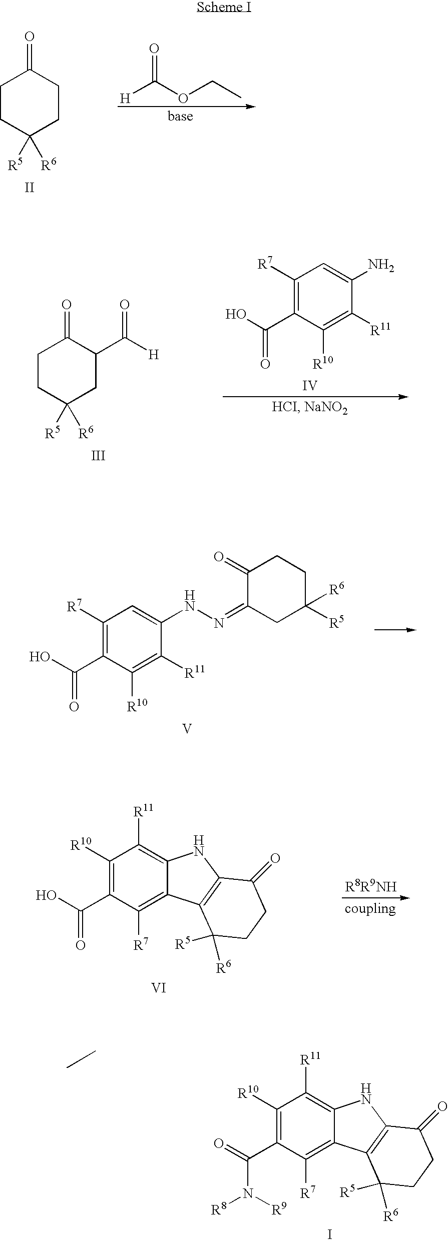

As illustrated in Scheme I an optionally substituted cyclohexanone is reacted with ethyl formate in the presence of a suitable base such as sodium hydride to provide the ketoaldehyde III. Reaction of III with a diazonium salt formed by reaction of aminobenzoic acid IV with acid and sodium nitrite provides the hydrazinobenzoic acid V. Heating V with formic acid provides VI. Amide coupling of the carboxylic acid on VI with the desired amine R₈R₉NH provides the desired compound of formula I. Standard peptide coupling reactions known in the art (see for example M. Bodanszky, 1984, The Practice of Peptide Synthesis, Springer-Verlag) may be employed in these syntheses. An example of suitable coupling conditions is treatment of a solution of VII in a suitable solvent such as DMF with HATU, HOAt, and a base such as diisopropylethylamine, followed by the desired amine R⁸R⁹NH. Further modification of the initial product of formula I by methods known in the art and illustrated in the Examples below may be used to prepare additional compounds of formula I.

Compounds of formula (I) having X═NH, R¹²=oxo and R¹³ not present may be prepared as illustrated in Scheme II.

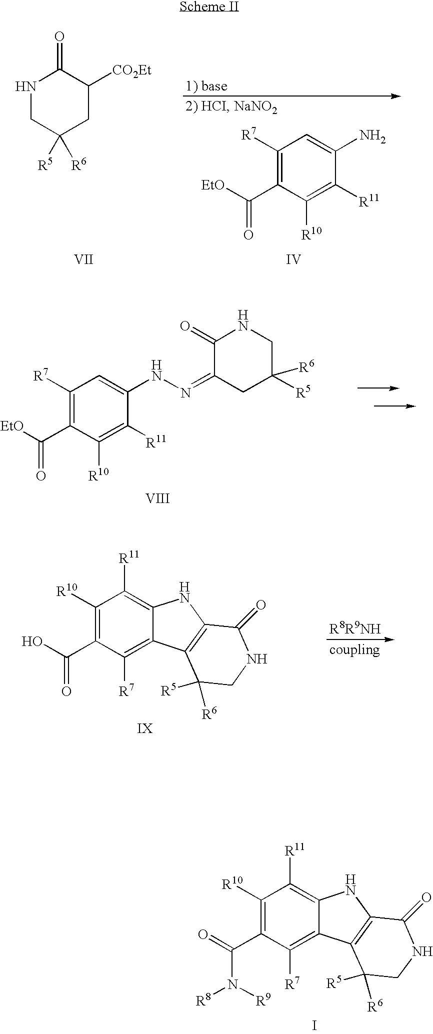

As shown in Scheme II an optionally substituted 2-oxopiperidine carboxylic acid ester is treated with base, followed by a diazonium salt formed by reaction of aminobenzoic acid IV with acid and sodium nitrite providing the hydrazinobenzoic acid ester VIII. Heating VIII with formic acid followed by hydrolysis of the ester provides IX. Amide coupling as described in Scheme I provides the desired compound of formula I.

An alternate procedure for preparing compounds of formula I having X═NH is illustrated in Scheme III.

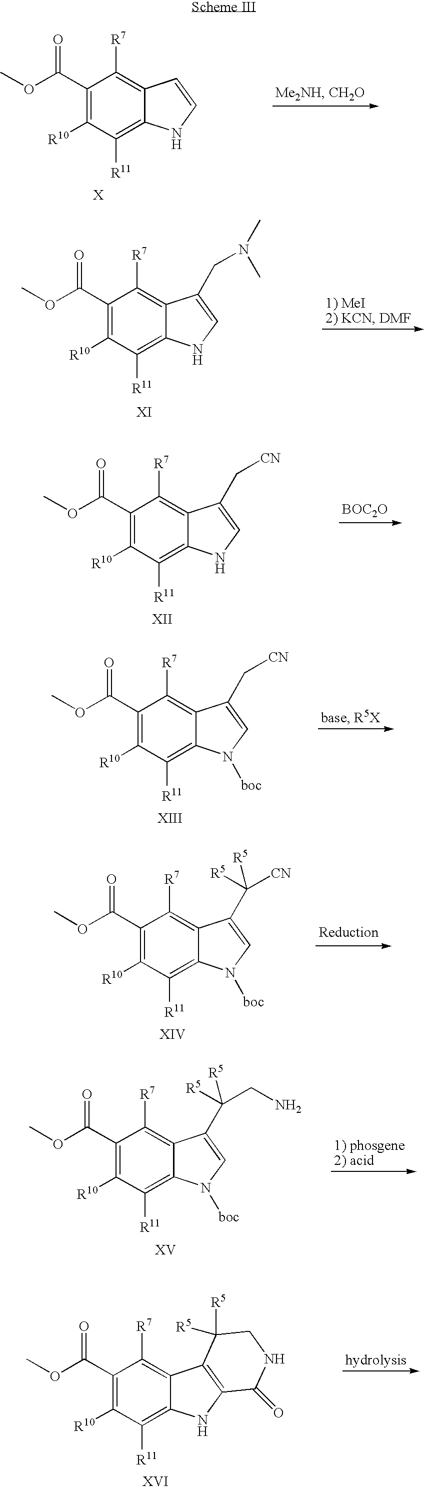

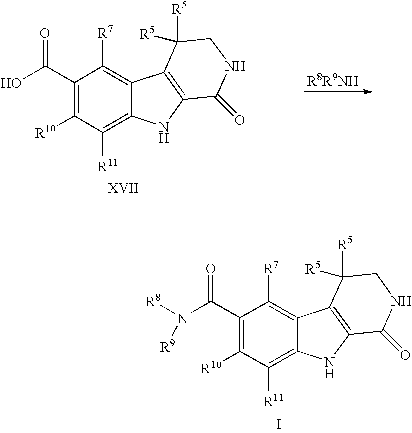

As illustrated in Scheme III an optionally substituted indole ester X is reacted with dimethylamine and formaldehyde to provide the dimethylaminomethylindole XI. Quarternization of the amine with methyl iodide followed by displacement with KCN provide nitrile XII. Protection of the indole nitrogen, for example with a Boc group as shown followed by alkylation with R⁵X where X is a leaving group such as I or Br, in the presence of a suitable base such as sodium hydride provides XIV. Reduction of the nitrile, for example by treatment with hydrogen in the presence of a suitable catalyst such as PtO₂ provides XV. Treatment of XV with phosgene followed by treatment with acid to remove the Boc protecting group provides XVI. Hydrolysis of XVI followed by amide coupling as described in Scheme I provides the desired compound of formula I.

Compounds of formula I having X═NH, R¹²=oxo and R¹³ not present and a substituent at R³ may be prepared as illustrated in Scheme IV.

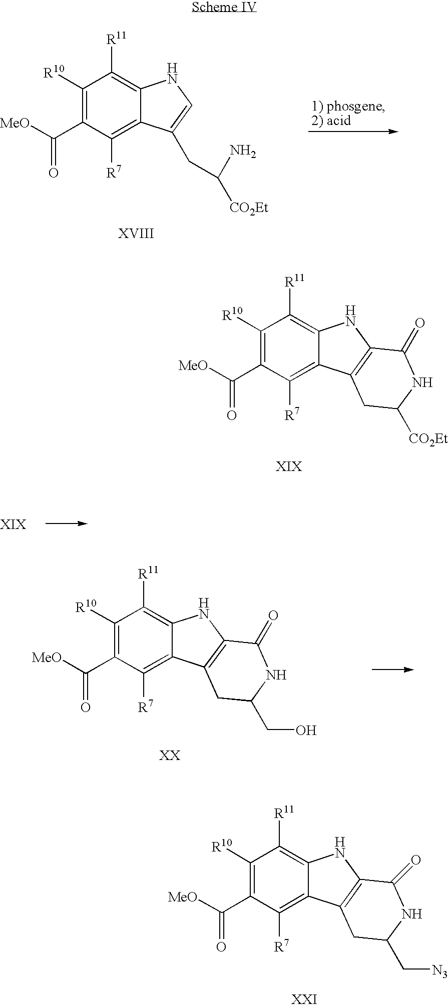

As illustrated above in Scheme IV, treatment of amino ester XVII with phosgene followed by acid provodes XIX bearing an ester at R³. The ester may be converted to other carboxylic acid derivatives or functional groups by methods known in the art. For example reduction of the ester with a suitable reducing agent such as lithium borohydride provides the hydroxymethyl derivative XX. Conversion of the hydroxyl to a leaving group for example a mesylate, followed by treatment with sodium azide provides azide XXI. The azide may be reduced by methods known in the art to provide an aminomethyl group at R³. Compounds of formula I may be prepared from these intermediates by hydrolysis and amide coupling as described in the schemes above. Several illustrative examples of these general methods are provided in the following section.

### SYNTHETIC EXAMPLES

### Example 1

### Synthesis of 4-methyl-1-oxo-2,3,4,9-tetrahydro-1H-β-carboline-6-carboxylic acid

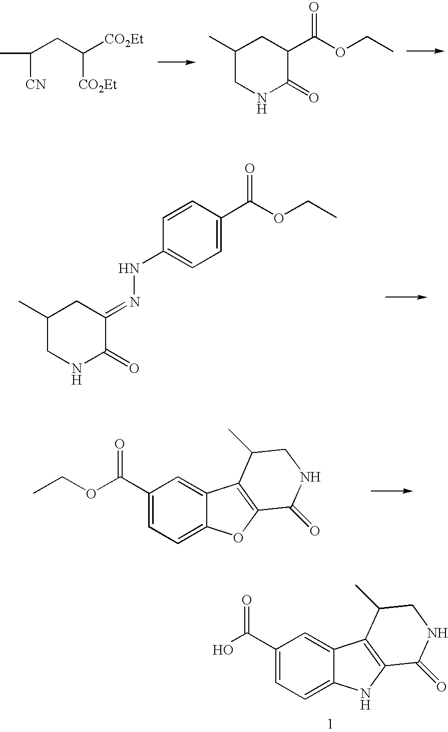

Sodium metal (2.0 g, 86.9 mmol) was added in portions to absolute ethanol (200 mL) under an N₂ atmosphere. Methacrylonitrile (36.0 g, 537 mmol) was added slowly at 0° C. and let stir for 30 min. Diethyl malonate (186 g, 1.16 mol) was added dropwise taking care not to let the reaction temperature rise above 35° C. The reaction mixture was stirred an additional 24 h at ambient temperature. Acetic acid (6.0 g, 100 mmol) was added to neutralize the reaction mixture. After diluting with water (200 mL), the reaction mixture was extracted with diethyl ether (2×500 mL). The combined organic extracts were washed with water (2×200 mL) and brine (200 mL), dried (MgSO₄) and concentrated under reduced pressure. The residue was distilled under reduced pressure to give 2-(2-cyano-2-methyl-ethyl)-malonic acid diethyl ester (101.3 g, 446 mmol, 83% yield) as a pale yellow oil. APCI-MS m/z 228 [M+H]⁺.

To a pressure-safe reaction vessel containing a solution of 2-(2-cyano-2-methyl-ethyl)-malonic acid diethyl ester (15.0 g, 66.0 mmol) in absolute ethanol (150 mL) was added platinum oxide (750 mg, 3.3 mmol). The vessel was pressurized with hydrogen gas (50 psi) and shaken for 56 h. The ethanol solution was passed through diatomaceous earth and washed with additional ethanol (2×400 mL) and the combined filtrates were concentrated under reduced pressure to provide a clear oil. Addition of hexanes afforded a white precipitate which was collected by filtration to yield 5-Methyl-2-oxo-piperidine-3-carboxylic acid ethyl ester (9.03 g, 48.8 mmol, 74% yield). APCI-MS m/z 186 [M+H]⁺.

5-Methyl-2-oxo-piperidine-3-carboxylic acid ethyl ester (15.9 g, 85.8 mmol) was treated with potassium hydroxide (6.26 g, 111.5 mmol) in water (150 mL) with stirring at ambient temperature for 18 h resulting in a clear solution. To a separate mixture of 4-amino-benzoic acid ethyl ester (19.8 g, 120.1 mmol) in water (200 mL) was added concentrated HCl (40 mL) and this mixture was cooled to 0° C. A solution of sodium nitrite (9.1 g, 133.0 mmol) in water (150 mL) was added dropwise to the 4-amino-benzoic acid ethyl ester solution. After the addition, the mixture was stirred for an additional 30 min. This entire solution was then added dropwise to the above solution at 0° C. This mixture was stirred for 30 min and the pH was adjusted to 5 with the addition of a saturated sodium bicarbonate solution. The reaction was stirred for an additional 4 h at 0° C. during which time an orange precipitate formed. 4-[N′-(5-methyl-2-oxo-piperidin-3-ylidene)-hydrazino]-benzoic acid ethyl ester (21.4 g, 73.9 mmol, 86% yield) was obtained by filtration as an orange solid. APCI-MS m/z 290 [M+H]⁺.

4-[N′-(5-Methyl-2-oxo-piperidin-3-ylidene)-hydrazino]-benzoic acid ethyl ester (14.0 g, 48.3 mmol) was diluted with formic acid (100 mL) and heated to reflux for 5 h. The dark colored reaction mixture was cooled to ambient temperature, diluted with water (250 mL) and extracted with methylene chloride (2×250 mL). The combined organic extracts were washed with water (2×100 mL) and brine (200 mL), dried (MgSO₄) and concentrated under reduced pressure. The residue was triturated with methylene chloride to afford 4-methyl-1-oxo-2,3,4,9-tetrahydro-1H—-carboline-6-carboxylic acid ethyl ester (7.3 g, 26.8 mmol, 55% yield) as an off-white solid. APCI-MS m/z 273 [M+H]⁺.

4-Methyl-1-oxo-2,3,4,9-tetrahydro-1H-β-carboline-6-carboxylic acid ethyl ester (4.8 g, 17.6 mmol) was diluted with a 1:1 mixture of water and methanol (100 mL). To this solution was added lithium hydroxide (844 mg, 35.3 mmol) and the mixture was heated to 90° C. for 2 h. The mixture was cooled to ambient temperature, diluted with water (100 mL) and acidified with concentrated HCl. The precipitate was collected by filtration and dried under reduced pressure providing the title compound as a pale yellow solid (4.2 g, 17.3 mmol, 98% yield). APCI-MS m/z 245 [M+H]⁺.

### Example 2

### Synthesis of 4,4-Dimethyl-1-oxo-2,3,4,9-tetrahydro-1H-β-carboline-6-carboxylic acid

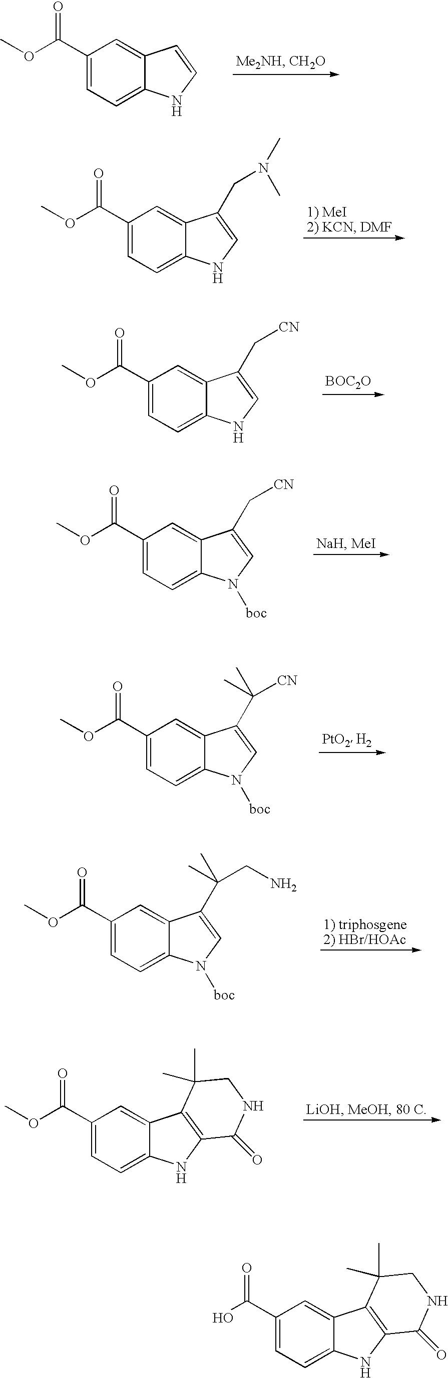

To a stirred solution of 37% formaldehyde (4.30 mL, 57 mmol) in HOAc-EtOH (1:1, 183 mL) at 0° C. was added 40% aq. Me₂NH (7.23 mL, 57 mmol). A solution of methyl indole-5-carboxylate (4.00 g, 23 mmol) in HOAc-EtOH (1:1, 57 mL) was added. The mixture was allowed to warm up to rt and stir overnight. Most volatiles were evaporated in vacuo. The residue was diluted with water (800 mL) and the pH was adjusted to 10-11 by adding 2N NaOH while cooling in an ice bath. The mixture was extracted with CH₂Cl₂ (3×200 mL). The combined extracts were dried over Na₂SO₄, filtered and concentrated in vacuo to give the crude product that was used directly in the next reaction.

To a solution of 3-Dimethylaminomethyl-1H-indole-5-carboxylic acid methyl ester (23 mmol) in THF (40 mL) at 0° C. was added dropwise CH₃I (1.7 mL, 27.40 mmol). The mixture was stirred at that temperature for 1.5 h and then concentrated in vacuo. The residue was mixed with DMF (40 mL). To the resulting mixture was added a solution of NaCN (4.48 g, 91.32 mmol) in water (20 mL). After stirring at 70° C. for 1.5 h, the reaction mixture was poured into ice water (400 mL). The mixture was extracted with EtOAc (500 mL). The organic layer was washed with water (2×120 mL), dried over NaSO₄, filtered and concentrated in vacuo. The residue was purified by combiflash (hexanes/ethyl acetate) to yield 3-Cyanomethyl-1H-indole-5-carboxylic acid methyl ester (2.73 g, 56% from compound 2) as a white solid.

To a mixture of 3-Cyanomethyl-1H-indole-5-carboxylic acid methyl ester (1.70 g, 7.9 mmol), Boc₂O (3.47 g, 15.9 mmol), triethylamine (2.22 mL, 16.00 mmol) and CH₂Cl₂ (7 mL) was added dimethylaminopyridine (49 mg, 0.4 mmol). Gas was evolved in a few minutes. After stirring at rt for 2 h, the reaction mixture was evaporated in vacuo. The residue was purified by combiflash (CH₂Cl₂) to afford 3-Cyanomethyl-indole-1,5-dicarboxylic acid 1-tert-butyl ester 5-methyl ester (2.18 g, 87%) as a white solid.

To a mixture of 3-Cyanomethyl-indole-1,5-dicarboxylic acid 1-tert-butyl ester 5-methyl ester (1.52 g, 4.84 mmol) and DMF (10 mL) at 0° C. was added NaH (60%, 484 mg, 12.1 mmol). The mixture was stirred at 0° C. for 30 min before CH₃I (0.9 mL, 14.5 mmol) was added. The mixture was stirred from 0° C. to rt over 3 h and then partitioned between EtOAc (120 mL) and water (80 mL). EtOAc phase was washed with water (2×50 mL), dried over NaSO₄, filtered and concentrated in vacuo. The residue was purified by combiflash (hexanes/ethyl acetate) to yield 3-(Cyano-dimethyl-methyl)-indole-1,5-dicarboxylic acid 1-tert-butyl ester 5-methyl ester (1.49 g, 90%) as a white solid.

A mixture of 3-(Cyano-dimethyl-methyl)-indole-1,5-dicarboxylic acid 1-tert-butyl ester 5-methyl ester (580 mg, 1.70 mmol), PtO₂ (39 mg, 0.17 mmol) and EtOH (8 mL) was hydrogenated under H₂ (50 psi) for 2 days. The resulting mixture was passed through a celite pad and the filtrate was evaporated in vacuo to afford 3-(2-Amino-1,1-dimethyl-ethyl)-indole-1,5-dicarboxylic acid 1-tert-butyl ester 5-methyl ester (580 mg, 99%) as a white solid.

To a solution of 3-(2-Amino-1,1-dimethyl-ethyl)-indole-1,5-dicarboxylic acid 1-tert-butyl ester 5-methyl ester (1.36 g, 3.9 mmol) in CH₂Cl₂ (20 mL) at 0° C. was added triphosgene (770 mg, 2.6 mmol) and TEA (0.6 mL, 4.32 mmol) successively. The mixture was stirred at rt for 30 min. Solvents were evaporated in vacuo and the residue was mixed with dry CH₃CN (20 mL). With stirring, 30% HBr/HOAc was added. The resulting mixture was refluxed for 1 h. Volatiles were evaporated in vacuo. The residue was purified by combiflash (hexanes/EtOAc) to yield 4,4-Dimethyl-1-oxo-2,3,4,9-tetrahydro-1H-β-carboline-6-carboxylic acid methyl ester (460 mg, 38%) as a white solid.

A mixture of 4,4-Dimethyl-1-oxo-2,3,4,9-tetrahydro-1H-β-carboline-6-carboxylic acid methyl ester (110 mg, 0.38 mmol), LiOH.H₂O (18 mg, 0.43 mmol), MeOH (2 mL), THF (0.7 mmol) and water (0.7 mmol) was stirred at 80° C. for 3 h. The mixture was concentrated in vacuo. The residue was acidified to pH 4 by adding 1N HCl. The precipitate was filtered to give title compound (93 mg, 89%) as a white solid.

### Example 3

### Synthesis of 1-oxo-2,3,4,9-tetrahydro-1H-β-carboline-6-carboxylic acid

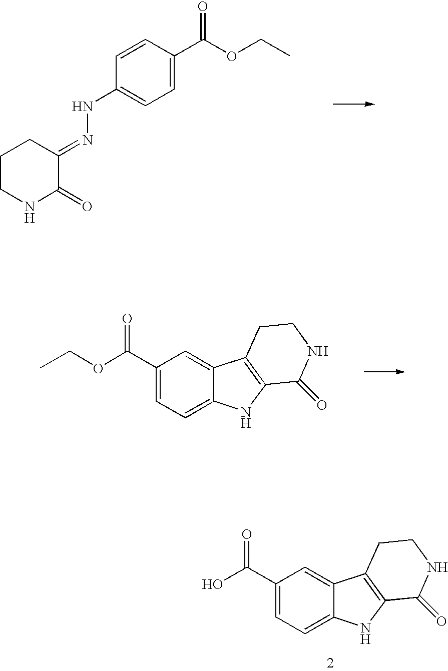

2-Oxo-piperidine-3-carboxylic acid ethyl ester (10.0 g, 58.4 mmol) was treated with potassium hydroxide (4.26 g, 75.9 mmol) in water (150 mL) with stirring at ambient temperature for 18 h resulting in a clear solution. To a separate mixture of 4-amino-benzoic acid ethyl ester (13.5 g, 81.8 mmol) in water (150 mL) was added concentrated HCl (30 mL) and this mixture was cooled to 0° C. A solution of sodium nitrite (6.2 g, 90.5 mmol) in water (150 mL) was added drop wise to the 4-amino-benzoic acid ethyl ester solution. After the addition, the mixture was stirred for an additional 30 min. This entire solution was then added dropwise to the above solution at 0° C. This mixture was stirred for 30 min and the pH was adjusted to 5 with the addition of a saturated sodium bicarbonate solution. The reaction was stirred for an additional 4 h at 0° C. during which time an orange precipitate formed. 4-[N′-(2-Oxo-piperidin-3-ylidene)-hydrazino]-benzoic acid ethyl ester (8.5 g, 30.9 mmol, 53% yield) was obtained by filtration as an orange solid. APCI-MS m/z 276 [M+H]⁺.

4-[N′-(2-Oxo-piperidin-3-ylidene)-hydrazino]-benzoic acid ethyl ester (6.8 g, 24.7 mmol) was diluted with formic acid (50 mL) and heated at refluxing temperature for 5 h. The dark colored reaction mixture was cooled to ambient temperature, diluted with water (250 mL) and extracted with methylene chloride (2×250 mL). The combined organic extracts were washed with water (2×100 mL) and brine (200 mL), dried (MgSO₄) and concentrated under reduced pressure. The residue was triturated with methylene chloride to afford 1-oxo-2,3,4,9-tetrahydro-1H-β-carboline-6-carboxylic acid ethyl ester (4.4 g, 17.1 mmol, 69% yield) as an off-white solid. APCI-MS m/z 259 [M+H]⁺.

1-Oxo-2,3,4,9-tetrahydro-1H-β-carboline-6-carboxylic acid ethyl ester (3.0 g, 11.6 mmol) was diluted with a 1:1 mixture of water and methanol (150 mL). To this solution was added lithium hydroxide (556 mg, 23.2 mmol) and the mixture was heated to 90° C. for 2 h. The mixture was cooled to ambient temperature, diluted with water (100 mL) and acidified with concentrated HCl. The precipitate is collected by filtration and washed with additional water. The solid was dried under reduced pressure providing the title compound 2 as a pale yellow solid (2.4 g, 10.5 mmol, 91% yield). APCI-MS m/z 231 [M+H]⁺.

### Example 4

### Synthesis of 8-oxo-6,7,8,9-tetrahydro-5H-carbazole-3-carboxylic acid pyridin-3-ylamide

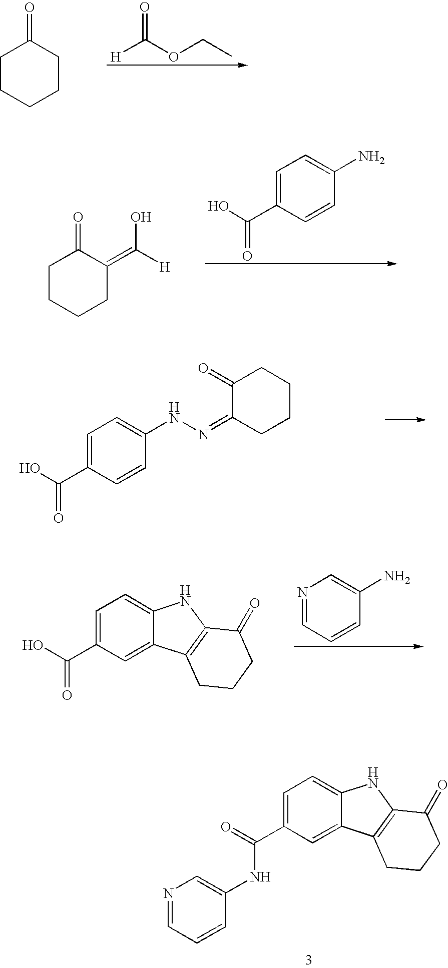

NaH (60% dispersion in oil, 1.76 g, 40.8 mmol) was suspended in ether (80 mL) and ethanol (0.21 mL) and cooled to 0° C. in an ice water bath. A solution of cyclohexanone (4.22 mL, 40.8 mmol) and ethyl formate (4.94 mL, 61.1 mmol) in ether (40 mL) was slowly added over 1 h. The stirred suspension was allowed to warm to room temperature overnight. Ethanol (5 mL) was then added and the reaction stirred for 1 h. The suspension was diluted with water (100 mL) and the organic layer separated. The aqueous phase was acidified with 6N HCl and extracted with Et₂O (3×100 mL). The combined organic extracts were washed with water, brine and dried with MgSO₄. The mixture was filtered and concentrated to provide 4.51 g of 2-oxo-cyclohexanecarboxaldehyde as a yellow oil.

To a 0° C. solution 2-oxo-cyclohexanecarboxaldehyde (3.22 g, 25.5 mmol) in MeOH (100 mL) was added NaOAc (4.89 g, 87.0 mmol) and water (100 mL). In a separate reaction vessel 4-amino-benzoic acid (3.50 g, 25.5 mmol) was taken up in water (100 mL) and concentrated HCl (7.2 mL) was added. The solution was cooled to 0° C. and a saturated solution of NaNO₂ (3.06 g, 51.0 mmol) was slowly added. Following addition, the diazotized solution was added dropwise via addition funnel to the stirred 2-oxo-cyclohexanecarbaldehyde solution over 30 min. The yellow suspension was allowed to stir for another 30 min and the solid precipitate filtered off. The solid was washed with water and dried in a vacuum oven overnight to provide 4.5 g of 4-{N′-[2-oxo-cyclohexylidene]-hydrazino}-benzoic acid as a yellow solid.

4-{N′-[2-oxo-cyclohexylidene]-hydrazino}-benzoic acid (1.00 g, 4.06 mmol) was dissolved in formic acid (100 mL) and heated to 100° C. for 12 h. The reaction was cooled and poured into water (300 mL). The resulting precipitate was filtered, washed with water and dried in a vacuum oven overnight to give 650 mg of 8-oxo-6,7,8,9-tetrahydro-5H-carbazole-3-carboxylic acid as a brown solid, which was used without further purification.

To a solution of 8-oxo-6,7,8,9-tetrahydro-5H-carbazole-3-carboxylic acid (0.2 g, 0.872 mmol) in DMF (5 mL) was added HATU (0.23 g, 0.96 mmol), DMAP (0.11 g, 0.87 mmol) and 3-aminopyridine (0.10 g, 1.04 mmol). The reaction was heated at 80° C. for 12 h and then poured into water (200 mL). The resulting precipitate was filtered, washed with water and triturated twice with MeOH to give 0.03 g of the title compound 3 as a white solid. ES-MS m/z 306 [M+H]⁺.

The following compounds were prepared by the method illustrated above in Example 3, using the appropriate amine in place of 2-aminopyridine for the final coupling step:
8-Oxo-6,7,8,9-tetrahydro-5H-carbazole-3-carboxylic acid (3-cyano-phenyl)-amide 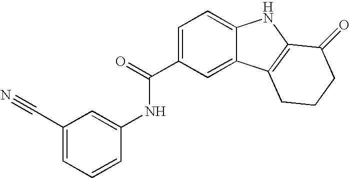
   prepared from 8-oxo-6,7,8,9-tetrahydro-5H-carbazole-3-carboxylic acid and 3-amino-benzonitrile. ES-MS m/z 330 [M+H]⁺.
8-Oxo-6,7,8,9-tetrahydro-5H-carbazole-3-carboxylic acid quinolin-3-ylamide 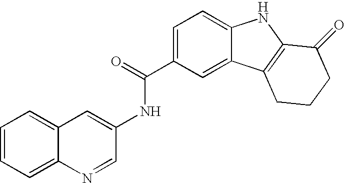
   prepared from 8-oxo-6,7,8,9-tetrahydro-5H-carbazole-3-carboxylic acid and quinolin-3-ylamine. ES-MS m/z 356 [M+H]⁺.
8-Oxo-6,7,8,9-tetrahydro-5H-carbazole-3-carboxylic acid thiazol-2-ylamide 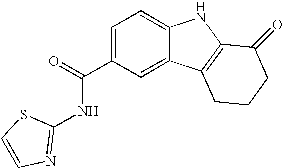
   prepared from 8-oxo-6,7,8,9-tetrahydro-5H-carbazole-3-carboxylic acid and thiazol-2-ylamine. ES-MS m/z 312 [M+H]⁺.

### Example 5

### Synthesis of 8-hydroxy-6,7,8,9-tetrahydro-5H-carbazole-3-carboxylic acid pyridin-3-ylamide

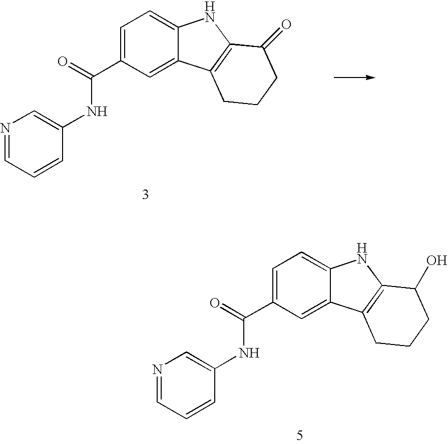

8-Oxo-6,7,8,9-tetrahydro-5H-carbazole-3-carboxylic acid (Example 3) (0.1 g, 0.33 mmol) was taken up in MeOH (5 mL) under N₂ and NaBH₄ (0.02 g, 0.49 mmol) added. The reaction was stirred at room temperature overnight and quenched with water (20 mL). The resulting precipitate was collect by filtration and washed with water. The residue was then triturated twice with MeOH to give 0.015 g of the title compound as a white powder. ES-MS m/z 308 [M+H]⁺.

### Example 6

### Synthesis of 8-hydroxyimino-6,7,8,9-tetrahydro-5H-carbazole-3-carboxylic acid pyridin-3-ylamide

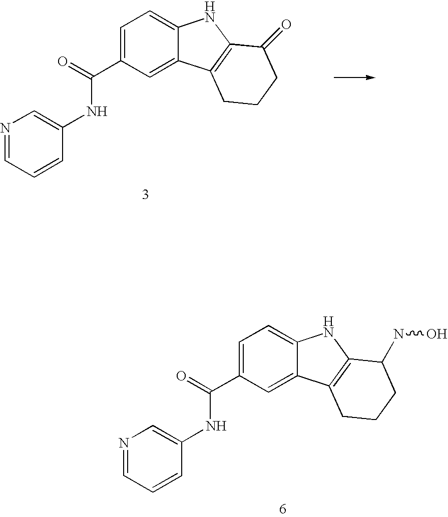

To a solution 8-oxo-6,7,8,9-tetrahydro-5H-carbazole-3-carboxylic acid pyridin-3-ylamide (Example 3) (0.15 g, 0.49 mmol) and NH₂OH.HCl (0.041 g, 0.59 mmol) in MeOH (5 mL) was added NaOAc (0.042 g, 0.59 mmol). The reaction was stirred under N₂ for 12 h and the solid precipitate filtered off. The precipitate was washed with MeOH and dried in a vacuum oven overnight to provide 0.10 g of 8-hydroxyimino-6,7,8,9-tetrahydro-5H-carbazole-3-carboxylic acid pyridin-3-ylamide as a 1:1 mixture or regioisomers. ES-MS m/z 321 [M+H]⁺.

The regioisomers were isolated by semi-preparative reverse phase HPLC purification of the above mixture. Removal of solvent provided the desired regioisomers:
8-[(E)-Hydroxyimino]-6,7,8,9-tetrahydro-5H-carbazole-3-carboxylic acid pyridin-3-ylamide; ES-MS m/z 321 [M+H]⁺. 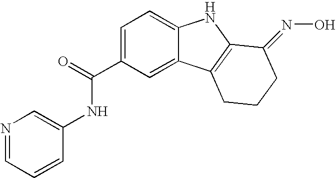
8-[(Z)-Hydroxyimino]-6,7,8,9-tetrahydro-5H-carbazole-3-carboxylic acid pyridin-3-ylamide; ES-MS m/z 321 [M+H]⁺.

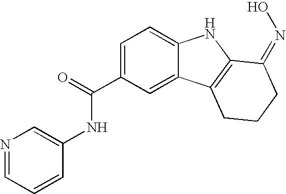

### Example 7

### Synthesis of 8-methyl-hydrazono-6,7,8,9-tetrahydro-5H-carbazole-3-carboxylic acid pyridin-3-ylamide

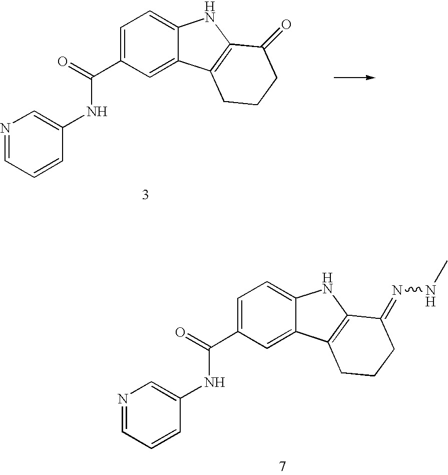

To a solution 8-oxo-6,7,8,9-tetrahydro-5H-carbazole-3-carboxylic acid pyridin-3-ylamide (0.10 g, 0.33 mmol) in MeOH (5 mL) was added methyl hydrazine (0.017 mL, 0.33 mmol) and the mixture was heated to 50° C. for 12 h. The solvent was removed and the residue treated with water (20 mL). The precipitate was collected by filtration and dried in a vacuum oven overnight to give 0.035 g of the title compound 7 as a yellow powder. ES-MS m/z 334 [M+H]⁺.

### Example 8

### Synthesis of 5,5-dimethyl-8-oxo-6,7,8,9-tetrahydro-5H-carbazole-3-carboxylic acid pyridin-3-ylamide

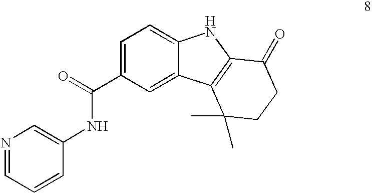

5,5-Dimethyl-8-oxo-6,7,8,9-tetrahydro-5H-carbazole-3-carboxylic acid pyridin-3-ylamide was prepared using the same procedure as Example 3 but starting with 4,4-dimethyl-cyclohexanone and ethyl formate. ES-MS m/z 334 [M+H]⁺.

### Example 9

### Synthesis of 5,5-dimethyl-8-oxo-6,7,8,9-tetrahydro-5H-carbazole-3-carboxylic acid thiazol-2-ylamide

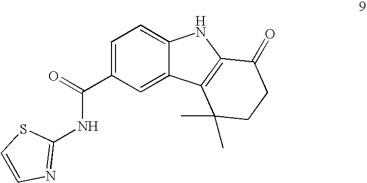

5,5-Dimethyl-8-oxo-6,7,8,9-tetrahydro-5H-carbazole-3-carboxylic acid thiazol-2-ylamide was prepared using a coupling step as described in Example 3 from 5,5-dimethyl-8-oxo-6,7,8,9-tetrahydro-5H-carbazole-3-carboxylic acid and thiazol-2-ylamine. ES-MS m/z 340 [M+H]⁺.

### Example 10

### Synthesis of 8-hydroxyimino-5,5-dimethyl-6,7,8,9-tetrahydro-5H-carbazole-3-carboxylic acid pyridin-3-ylamide

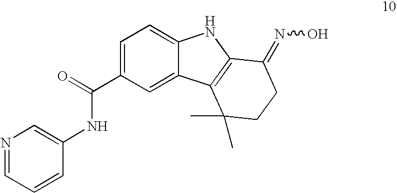

8-Hydroxyimino-5,5-dimethyl-6,7,8,9-tetrahydro-5H-carbazole-3-carboxylic acid pyridin-3-ylamide was prepared by the procedure described in Example 6 from hydroxylamine hydrochloride and 5,5-dimethyl-8-oxo-6,7,8,9-tetrahydro-5H-carbazole-3-carboxylic acid pyridin-3-ylamide. ES-MS m/z 349 [M+H]⁺.

### Example 11

### Synthesis of 8-hydroxyimino-5,5-dimethyl-6,7,8,9-tetrahydro-5H-carbazole-3-carboxylic acid thiazol-2-ylamide

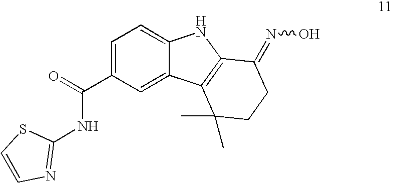

8-Hydroxyimino-5,5-dimethyl-6,7,8,9-tetrahydro-5H-carbazole-3-carboxylic acid thiazol-2-ylamide was prepared by the procedure described in Example 6 from hydroxylamine hydrochloride and 5,5-dimethyl-8-oxo-6,7,8,9-tetrahydro-5H-carbazole-3-carboxylic acid thiazol-2-ylamide. The product was purified by semi-prep reverse phase HPLC and isolated as a white solid following removal of the solvent. ES-MS m/z 355 [M+H]⁺.

### Example 12

### Synthesis of 8-methoxyimino-5,5-dimethyl-6,7,8,9-tetrahydro-5H-carbazole-3-carboxylic acid thiazol-2-ylamide

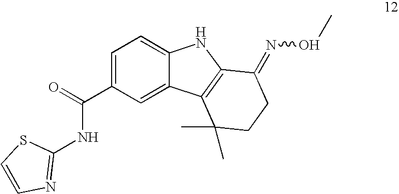

8-Methoxyimino-5,5-dimethyl-6,7,8,9-tetrahydro-5H-carbazole-3-carboxylic acid thiazol-2-ylamide was by the procedure described in Example 6 using 5,5-dimethyl-8-oxo-6,7,8,9-tetrahydro-5H-carbazole-3-carboxylic acid thiazol-2-ylamide (Example 9) and O-methyl hydroxylamine hydrochloride. ES-MS m/z 369 [M+H]⁺.

### Example 13

### Synthesis of 8-hydrazono-5,5-dimethyl-6,7,8,9-tetrahydro-5H-carbazole-3-carboxylic acid thiazol-2-ylamide

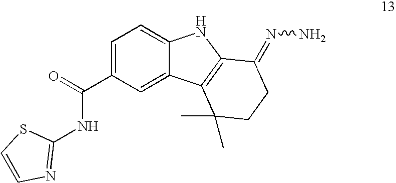

To a solution of 5,5-dimethyl-8-oxo-6,7,8,9-tetrahydro-5H-carbazole-3-carboxylic acid thiazol-2-ylamide (Example 9) (0.08 g, 0.24 mmol) in MeOH (5 mL) was added hydrazine monohydrate (0.047 mL, 0.96 mmol). The reaction was stirred at room temperature for 12 h and MgSO₄ was added. The mixture was filtered and the solvent removed to give 0.06 g of 8-hydrazono-5,5-dimethyl-6,7,8,9-tetrahydro-5H-carbazole-3-carboxylic acid thiazol-2-ylamide as a brown powder. ES-MS m/z 348 [M+H]⁺.

### Example 14

### Synthesis of 1-hydroxyimino-4-methyl-2,3,4,9-tetrahydro-1H-β-carboline-6-carboxylic acid pyridin-3-ylamide

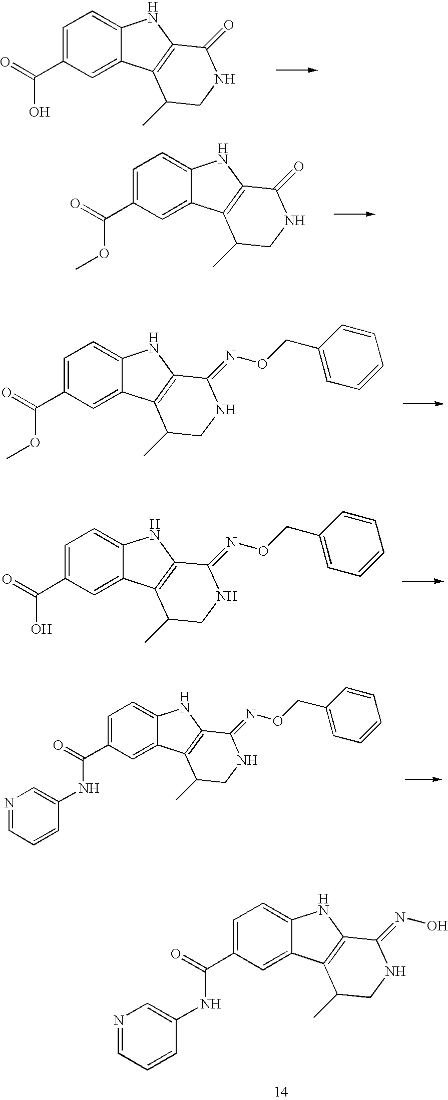

4-Methyl-1-oxo-2,3,4,9-tetrahydro-1H-β-carboline-6-carboxylic acid (Example 1) (0.4 g, 1.64 mmol) was dissolved in MeOH (40 mL) and saturated with HCl gas. The flask was then fitted with a reflux condenser and the reaction heated at 60° C. for 12 h. The mixture was cooled and the solvent removed under reduced pressure to give 0.38 g of 4-methyl-1-oxo-2,3,4,9-tetrahydro-1H-β-carboline-6-carboxylic acid methyl ester as a yellow powder.

4-Methyl-1-oxo-2,3,4,9-tetrahydro-1H-β-carboline-6-carboxylic acid methyl ester (0.2 g, 0.78 mmol) was dissolved in POCl₃ (1 mL) under N₂ and stirred for 12 h. Et₂O (10 mL) was then added and the yellow precipitate collected by filtration and washed with Et₂O (3×10 mL). The precipitate was suspended in dry MeCN (5 mL) and O-benzyl-hydroxylamine hydrochloride (0.15 g, 0.93 mmol) and Et₃N (0.26 mL, 1.86 mmol) were added. The reaction was stirred under N₂ at room temperature for 1 h and then heated to 60° C. for 12 h. The mixture was cooled, diluted with water (50 mL) and extracted with EtOAc (3×100 mL). The combined organic extracts were washed with brine, dried with MgSO₄, filtered and concentrated. The residue was purified by chromatography to provide 0.23 g of 1-benzyloxyimino-4-methyl-2,3,4,9-tetrahydro-1H-β-carboline-6-carboxylic acid methyl ester as a white powder.

The above methyl ester (0.15 g, 0.41 mmol) was dissolved in THF (2 mL) and water (2 mL). 2 M NaOH (0.21 mL, 0.42 mmol) was then added and the reaction heated to 60° C. for 12 h. The mixture was cooled and acidified with 6 N HCl. The residue was extracted with EtOAc (3×100 mL) and the combined extracts where washed with water, brine, dried with MgSO₄ and filtered. The solvent was removed to give 1-benzyloxyimino-4-methyl-2,3,4,9-tetrahydro-1H-β-carboline-6-carboxylic acid as a white powder, which was used without further purification.

To a solution of the above carboxylic acid (0.19 g, 0.54 mmol) in DMF (2 mL) was added HATU (0.62 g, 1.63 mmol), DMAP (0.2 g, 1.63 mmol) and pyridine-3-ylamine (0.10 g, 1.09 mmol). The reaction was heated at 80° C. for 12 h and then poured into water (200 mL). The resulting precipitate was filtered and taken up in EtOAc (100 mL). The organic phase was washed with water, brine, dried with MgSO₄ and filtered. The solvent was removed and the residue purified by chromatography to give 0.065 g of 1-benzyloxyimino-4-methyl-2,3,4,9-tetrahydro-1H-β-carboline-6-carboxylic acid pyridine-3-ylamide as a white solid.

The above amide (0.04 g, 0.094 mmol) was dissolved in MeOH (5 mL) and 10% Pd/C (0.1 g) added. H₂ gas was passed over the suspension for 12 h and then filtered through diatomaceous earth. The filtrate was concentrated to give 0.016 g of the title compound as a white powder. ES-MS m/z 335 [M+H]⁺.

### Example 15

### Synthesis of 1-methoxyimino-4-methyl-2,3,4,9-tetrahydro-1H-β-carboline-6-carboxylic acid pyridin-3-ylamide

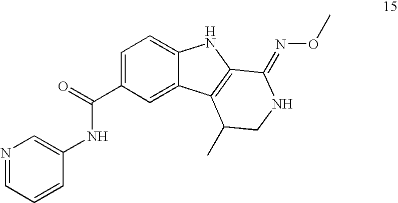

1-Methoxyimino-4-methyl-2,3,4,9-tetrahydro-1H-β-carboline-6-carboxylic acid pyridin-3-ylamide was prepared by the procedure described in Example 6 using 1-oxo-2,3,4,9-tetrahydro-1H-β-carboline-6-carboxylic acid methyl ester and O-methyl-hydroxylamine hydrochloride. ES-MS m/z 350 [M+H]⁺.

### Example 16

### Synthesis of 4-methyl-1-oxo-2,9-dihydro-1H-β-carboline-6-carboxylic acid pyridin-3-ylamide

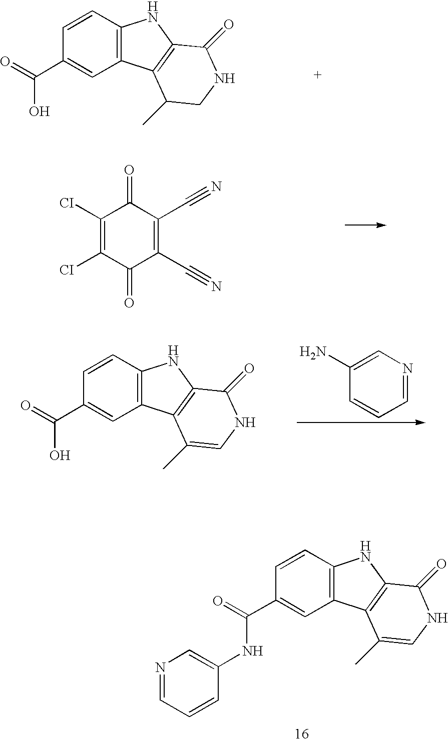

4-Methyl-1-oxo-2,3,4,9-tetrahydro-1H-β-carboline-6-carboxylic acid (500 mg, 2.05 mmol) (Example 1) and DDQ (795 mg, 3.50 mmol) were suspended in dioxane (15 mL) and heated to 80° C. for 20 h. The reaction was cooled, diluted with water (25 mL) and the solids filtered. The solids were washed copiously with water, then MeCN, and dried in a vacuum oven at 60° C. for 12 h to give 4-methyl-1-oxo-2,9-dihydro-1H-β-carboline-6-carboxylic acid as an off-white solid (375 mg, 76%).

To a solution of the above carboxylic acid (80 mg, 0.33 mmol) in anhydrous DMF (3 mL) was added HATU (400 mg, 1.05 mmol), DMAP (150 mg, 1.23 mmol) and pyridine-3-ylamine (120 mg, 1.28 mmol). The reaction was stirred at room temperature for 16 h, then heated to 65° C. for 2 h. The reaction was poured into water (20 mL). The resulting precipitate was filtered, washed with water and triturated with hot MeOH to give the title compound (67 mg, 64%) as a white solid. ES-MS m/z 319 [M+H]⁺.

### Example 17

### Synthesis of 1-Oxo-2,9-dihydro-1H-β-carboline-6-carboxylic acid pyridin-3-ylamide

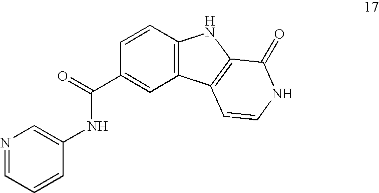

The title compound was prepared from 1-oxo-2,3,4,9-tetrahydro-1H-β-carboline-6-carboxylic acid (Example 2) by the procedure described in Example 16. ES-MS m/z 305 [M+H]⁺.

### Example 18

### Synthesis of 8-amino-6,7,8,9-tetrahydro-5H-carbazole-3-carboxylic acid pyridin-3-ylamide hydrochloride salt

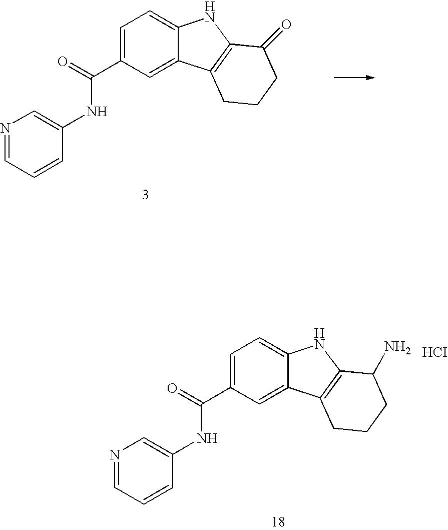

A solution of 8-oxo-6,7,8,9-tetrahydro-5H-carbazole-3-carboxylic acid pyridin-3-ylamide (Example 3) (325 mg, 1.06 mmol), ammonium acetate (700 mg), and sodium cyanoborohydride (900 mg) in MeOH (15 mL) was heated to 65° C. for 24 h. The reaction was cooled, quenched with concentrated HCl (to pH 1; CAUTION: HCN release), and concentrated in vacuo. The residue was made strongly alkaline with 4N KOH, and the resulting precipitate collected, washed with water, and dried in a vacuum oven. The crude product was then taken up in MeOH, filtered through a 0.45μ PTFE filter, and acidified with 4N HCl in dioxane (6 mL). Et₂O was added to precipitate the product which was filtered and dried in vacuo to give the title compound (326 mg, 92%) as a pale yellow powder. ES-MS m/z 307 [M+H]⁺.

### Example 19

### Synthesis of 8-ureido-6,7,8,9-tetrahydro-5H-carbazole-3-carboxylic acid pyridin-3-ylamide

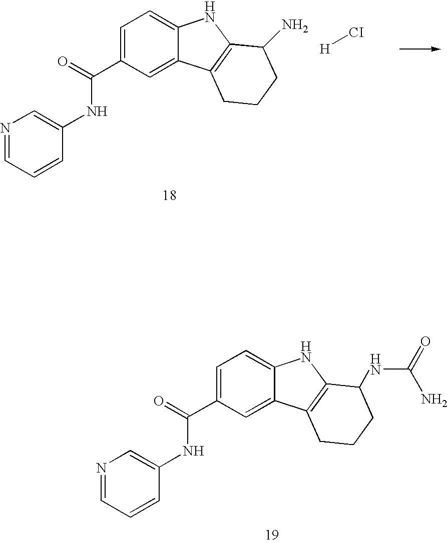

8-Amino-6,7,8,9-tetrahydro-5H-carbazole-3-carboxylic acid pyridin-3-ylamide hydrochloride salt (Example 18) (75 mg, 0.22 mmol), trimethylsilyl isosyanate (120 mg, 1.04 mmol), and pyridine (200 mg, 2.53 mmol) were combined in anhydrous DMF (2 mL) and stirred at 60° C. for 10 h. Water (1 mL) was added and the reaction was stirred at 60° C. for 2 h, then concentrated to dryness under reduced pressure. The residue was taken up in MeOH and purified by reverse phase preparatory HPLC (water/MeCN, 1% formic acid, C8 column) to provide the title compound as a white solid (26 mg, 34%) after lyophilization. ES-MS m/z 350 [M+H]⁺.

### Example 20

### Synthesis of 8-acetylamino-6,7,8,9-tetrahydro-5H-carbazole-3-carboxylic acid pyridin-3-ylamide

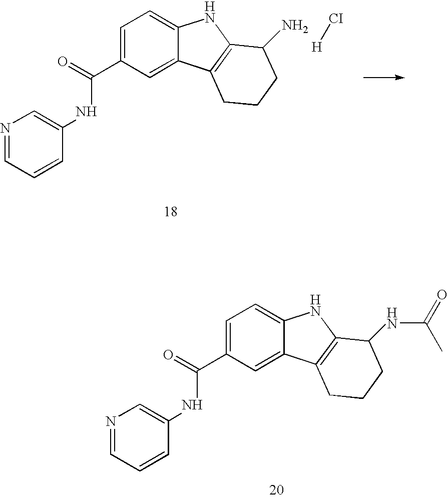

8-Amino-6,7,8,9-tetrahydro-5H-carbazole-3-carboxylic acid pyridin-3-ylamide hydrochloride salt (Example 18) (55 mg, 0.16 mmol), pyridine (500 μL), and acetyl chloride (500 μL) were combined in DMF (3 mL) and stirred at room temperature for 6 h. The reaction was quenched with water (1 mL) and MeOH (1 mL), then concentrated in vacuo. The residue was taken up in MeOH and purified by reverse phase preparatory HPLC (water/MeCN, 1% formic acid, C8 column) to provide the title compound as a tan solid (23 mg, 41%) after lyophilization. ES-MS m/z 349 [M+H]⁺.

### Example 21

### Synthesis of 1,5-dioxo-1,2,3,4,5,10-hexahydro-azepino[3,4-b]indole-7-carboxylic acid pyridin-3-ylamide

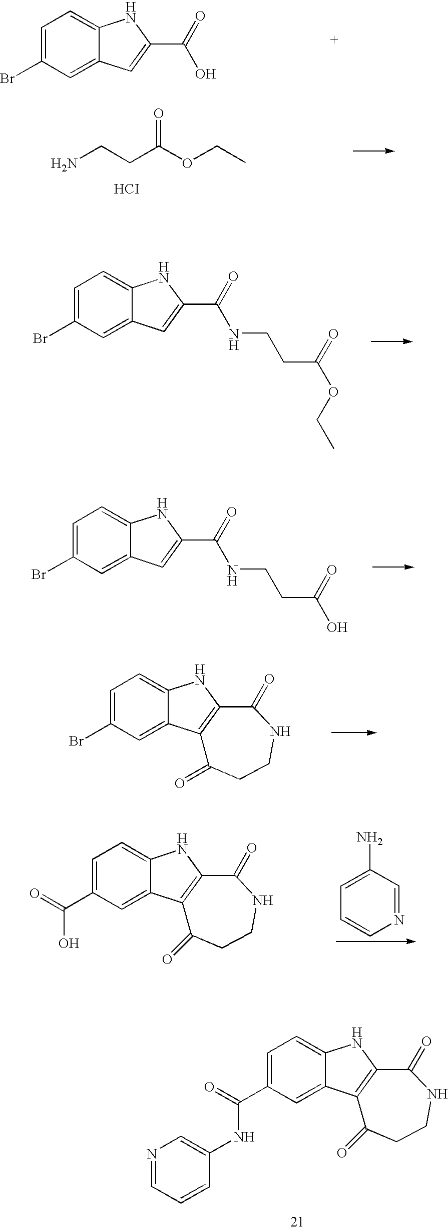

5-Bromoindole-2-carboxylic acid (6.20 g, 25.8 mmol) and DMAP (5.60 g, 45.8 mmol) were suspended in anhydrous methylene chloride (400 mL) under an atmosphere of nitrogen and cooled to 0° C. EDCI (6.20 g, 32.3 mmol) and β-alanine ethyl ester hydrochloride (4.77 g, 31.1 mmol) were added, and the reaction mixture stirred at 0° C. for 4 h, then at room temperature for 20 h. Water was added, and the organic layer separated and subsequently washed with 10% HCl, then dried over Na₂SO₄, filtered, and concentrated to give 3-[(5-Bromo-1H-indole-2-carbonyl)-amino]-propionic acid ethyl ester (7.21 g, 82%) as an analytically pure pale yellow powder.

The above ethyl ester (5.20 g, 15.3 mmol) and lithium hydroxide monohydrate (2.00 g, 47.7 mmol) were combined in 95% EtOH (300 mL) and stirred at room temperature for 20 h. The reaction mixture was concentrated, and the residue diluted with water (100 mL), and acidified to pH 3 with concentrated HCl. The resulting precipitate was collected, washed with water, and dried in a vacuum oven to give 3-[(5-Bromo-1H-indole-2-carbonyl)-amino]-propionic acid (4.12 g, 86%) as an analytically pure white powder.

Phosphorous pentoxide (3.73 g, 26.5 mmol) was suspended in methanesulfonic acid (150 mL) and heated to 7° C. in a sealed vessel until a clear solution was obtained. 3-[(5-Bromo-1H-indole-2-carbonyl)-amino]-propionic acid (4.12 g, 13.2 mmol) was then added, and the reaction heated to 11° C. for 1.5 h. The reaction was cooled, then poured into ice water and stirred for 1 h to allow the product to granulate. The solids were collected, washed copiously with water, then acetone, and dried in a vacuum oven to give 7-bromo-3,4-dihydro-2H,10H-azepino[3,4-b]indole-1,5-dione (3.47 g, 97%) as an analytically pure tan solid.

7-Bromo-3,4-dihydro-2H,10H-azepino[3,4-b]indole-1,5-dione (3.52 g, 12.0 mmol), palladium (II) acetate (150 mg, 0.668 mmol), dppf (200 mg, 0.361 mmol), and triethylamine (20 mL) were combined with anhydrous DMSO/MeOH (3:1) (200 mL) in a 300 mL Parr bomb. The apparatus was flushed with 100 psi carbon monoxide (three times) and then pressurized to 100 psi with carbon monoxide and sealed. The bomb was heated to 85° C. with stirring for 20 h and the reaction mixture was subsequently cooled, and concentrated under high vacuum. The dark residue was triturated with water and collected by suction filtration. These solids were suspended in a mixture of water (40 mL) and MeOH (200 mL) and to this was added lithium hydroxide monohydrate (9 g). The resulting suspension was stirred at 75° C. for 6 h, then filtered hot through diatomaceous earth followed by a rinse with 6 N KOH and MeOH. The filtrate was concentrated to a quarter of its original volume and diluted with water (100 mL). Concentrated HCl was added to make the solution strongly acidic, and the resulting suspension was allowed to cool with stirring overnight. The solids were filtered, washed copiously with water, and dried in a vacuum oven to give the crude product 1,5-dioxo-1,2,3,4,5,10-hexahydro-azepino[3,4-b]indole-7-carboxylic acid (3.12 g) as a brown solid. This was used in subsequent steps without further purification.

To a solution of 1,5-dioxo-1,2,3,4,5,10-hexahydro-azepino[3,4-b]indole-7-carboxylic acid (3.10 g, 12.0 mmol) in anhydrous DMF (10 mL) was added HATU (5.80 g, 15.3 mmol), DMAP (3.00 g, 25.0 mmol) and pyridine-3-ylamine (2.50 g, 26.6 mmol). The reaction was stirred at 60° C. for 24 h. The reaction was poured into water and the resulting precipitate was filtered, washed with water and triturated with cold MeOH to give the title compound (3.50 g) as a brown powder. This material was purified by reverse phase preparatory HPLC (water/MeCN, 1% formic acid, C8 column). ES-MS m/z 335 [M+H]⁺.

### Example 22

### Synthesis of 1-oxo-5-(pyridin-2-yl-hydrazono)-1,2,3,4,5,10-hexahydro-azepino[3,4-b]indole-7-carboxylic acid pyridin-3-ylamide

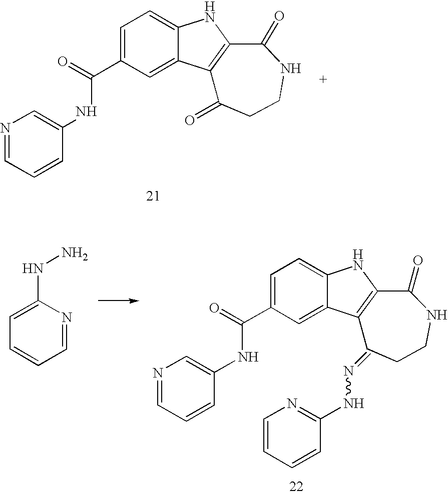

1,5-Dioxo-1,2,3,4,5,10-hexahydro-azepino[3,4-b]indole-7-carboxylic acid pyridin-3-ylamide (150 mg, 0.449 mmol) and 2-hydrazinopyridine (90 mg, 0.83 mmol) were dissolved in 1:1 AcOH/EtOH (2 mL) and heated to 100° C. for 24 h. The reaction was cooled, concentrated, and purified by reverse phase preparatory HPLC (water/MeCN, 1% formic acid, C8 column) to give the title compound (21 mg, 11%) as a brown powder after lyophilization. ES-MS m/z 426 [M+H]⁺.

### Example 23

### Synthesis of 5-hydroxyimino-1-oxo-1,2,3,4,5,10-hexahydro-azepino[3,4-b]indole-7-carboxylic acid pyridin-3-ylamide

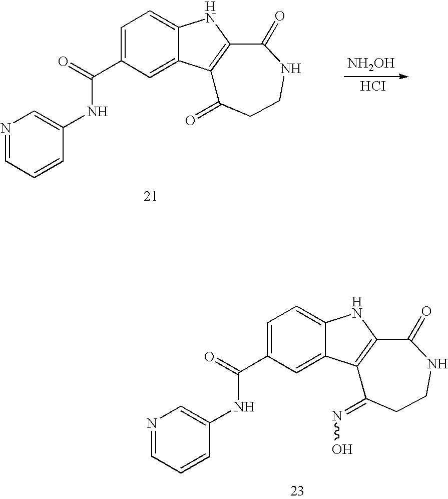

1,5-Dioxo-1,2,3,4,5,10-hexahydro-azepino[3,4-b]indole-7-carboxylic acid pyridin-3-ylamide (200 mg, 0.600 mmol), hydroxylamine hydrochloride (500 mg, 7.20 mmol), and pyridine (500 μL) were combined in MeOH (3 mL) and heated to 60° C. for 20 h. The reaction was then cooled, diluted with water, and filtered. The resulting solids were washed with water and purified by reverse phase preparatory HPLC (water/MeCN, 1% formic acid, C8 column) to give the title compound (17 mg, 8%) as a white foam. ES-MS m/z 349 [M+H]⁺.

### Example 24

### Synthesis of 4-methyl-1-oxo-2,3,4,9-tetrahydro-1H-β-carboline-6-carboxylic acid [4-(2-dimethylamino-ethylcarbamoyl)-phenyl]-amide

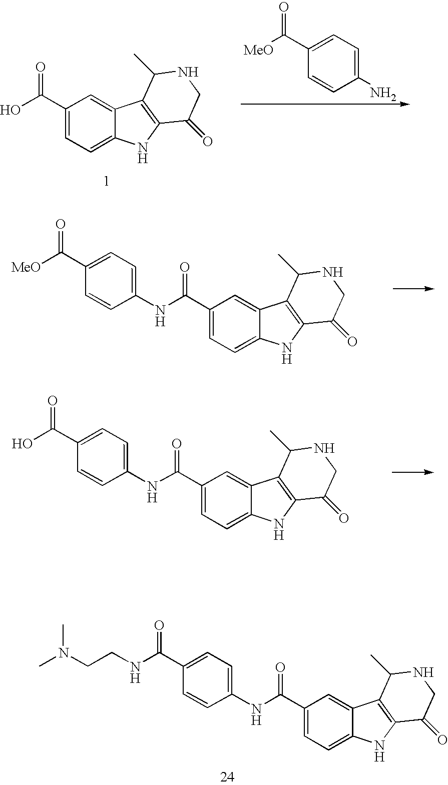

To a solution of 4-methyl-1-oxo-2,3,4,9-tetrahydro-1H-β-carboline-6-carboxylic acid (Example 1) (610 mg, 2.5 mmol.) and HOBT (372 mg, 2.7 mmol.) in DMF (12 mL) was added EDC (525 mg, 2.8 mmol.). The solution was stirred at rt for 2 h. Next, 4-aminobenzoic acid methyl ester (791 mg, 5.2 mmol), diisopropylethylamine (1.0 mL, 5.7 mmol.) and DMAP (60 mg, 0.5 mmol.) were added in sequence. The mixture was then heated to 6° C. while placed under a N₂ stream to slowly remove DMF overnight. The mixture was cooled to room temperature and MeOH was added. The yellowish precipitate was collected and washed with methanol to give the desired amide ester (895 mg, yield 95%).

To a suspension of the above amide ester (400 mg, 1.1 mmol.) in MeOH (10 mL) was added 10% KOH in 9:1 MeOH—H₂O (5 mL). The mixture was heated at 70° C. for 5 h. The mixture was cooled to room temperature and concentrated. The residue was re-dissolved in water (10 mL). The mixture was acidified with 6N HCl to pH ˜2. The white precipitate was collected by filtration to give the desired amide carboxylic acid (380 mg, yield 85%).

To a solution of the above amide carboxylic acid (40 mg, 0.11 mmol.) and HOBt (22 mg, 0.17 mmol.) in DMF (2 mL) was added EDC (32 mg, 0.17 mmol.), followed by N,N-dimethylethylenediamine (19 mg, 0.22 mL) and DMAP (3 mg, 0.022 mmol.). The mixture was stirred overnight. The residue was concentrated. A precipitate formed when MeOH was added. The solid was collected and washed with MeOH several times to yield the title compound (24 mg, yield 50%) as light yellow solid; ES-MS m/z 434 [M+H]⁺.

The following compound was prepared by the procedure described in the above example using 2-aminothiazole-4-carboxylic acid methyl ester in place of 4-aminobenzoic acid methyl ester:
4-Methyl-1-oxo-2,3,4,9-tetrahydro-1H-β-carboline-6-carboxylic acid [4-(3-methyl-butylcarbamoyl)-thiazol-2-yl]-amide. ES-MS m/z 441 [M+H]⁺.

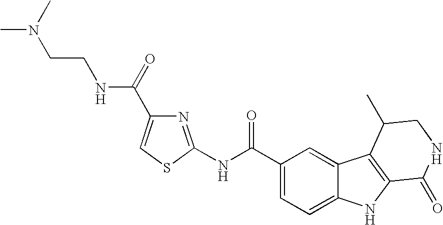

### Example 25

### Synthesis of 2-fluoro-4-[(4-methyl-1-oxo-2,3,4,9-tetrahydro-1H-β-carboline-6-carbonyl)-amino]-benzoic acid methyl ester

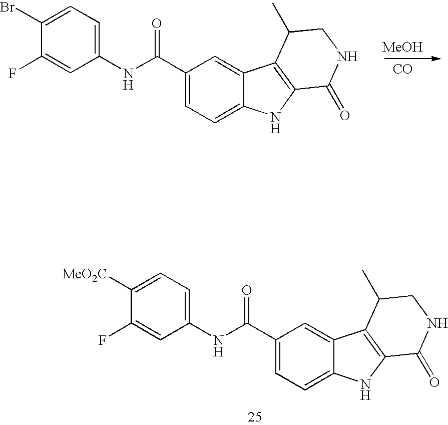

To a mixture of 4-methyl-1-oxo-2,3,4,9-tetrahydro-1H-β-carboline-6-carboxylic acid (4-bromo-3-fluoro-phenyl)-amide (100 mg, 0.24 mmol.), Pd(dppf)Cl₂ (25 mg, 0.03 mmol.), triethylamine (0.2 mL, 1.4 mmol.) and MeOH (2.5 mL) in DMF (10 mL) was passed CO gas for 3 min. The mixture was then heated to 85° C. in a sealed vial overnight. After cooling to room temperature, the mixture was concentrated. The residue was purified by silica gel chromatography using 0-20% MeOH-DCM to give 68 mg (72% yield) of the title compound. ES-MS m/z 396 [M+H]⁺.

### Example 26

### Synthesis of N-phenyl 4,4-spirocyclobutyl-1-oxo-2,3,4,9-tetrahydro-1H-1-carboline-6-carboxamide

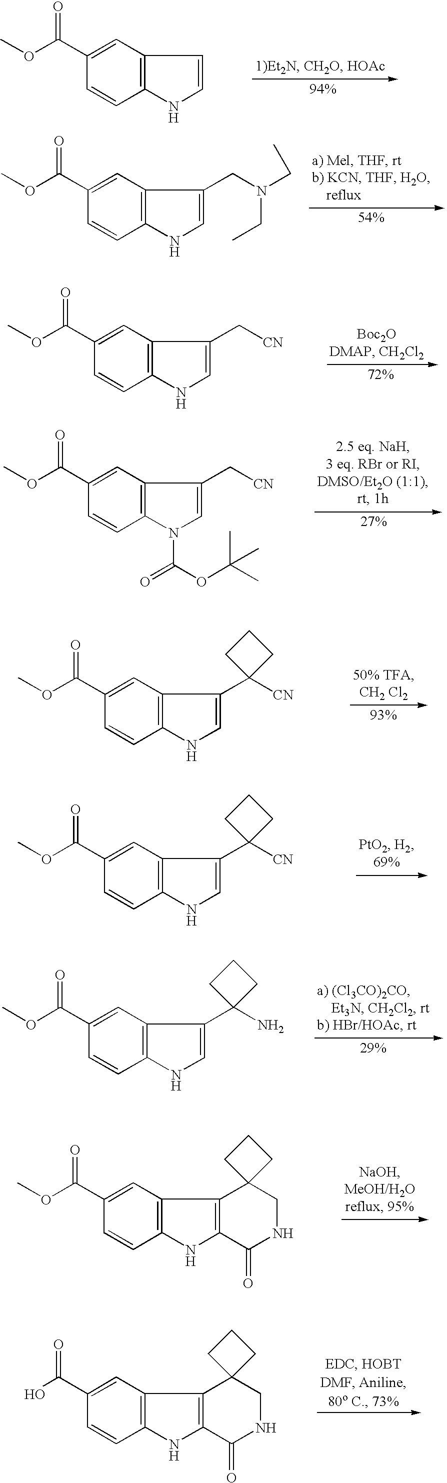

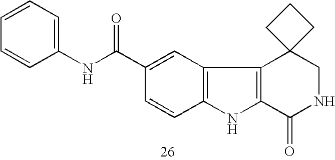

A reaction flask equipped with a nitrogen line and a stir bar was charged with acetic acid (8.1 mL, 142.1 mmol). The flask was cooled to 0° C. and diethylamine (10.6 mL, 102.7 mmol) was added dropwise over 30 min. Aqueous formaldehyde (37%, 6.7 mL, 90 mmol) was added, followed by methyl indole-5-carboxylate (15.0 g, 85.6 mmol). The reaction was allowed to warm slowly to room temperature, and was stirred for 1 h at room temperature. The reaction mixture was partitioned between 10% aqueous sodium hydroxide (250 mL) and dichloromethane. The organic layer was dried with anhydrous sodium sulfate. The solvent was evaporated to give 3-diethylaminomethyl-1H-indole-5-carboxylic acid methyl ester (21 g, 94%).

A reaction flask equipped with a nitrogen line and a mechanical stirrer was charged with the above methyl ester (21 g, 80.6 mmol) in THF (100 mL). The flask was cooled to 0° C. and iodomethane (6.0 mL, 96.8 mmol) was added. The reaction mixture was stirred for 24 h at room temperature. A solution of potassium cyanide (6.3 g, 96.8 mmol) in water (5 mL) was added. The reaction was refluxed for 5 h. The solvent was removed and the residue was dissolved in dichloromethane (300 mL). The organic layer was washed in turn with water (2×100 mL), 5% aqueous hydrochloric acid (100 mL) and brine (100 mL). The organic layer was dried over anhydrous sodium sulfate. The solvent was evaporated and the resulting oil was purified by chromatography on a Combiflash system eluting with an EtOAc/dichloromethane gradient (0-20%) to give 3-cyanomethyl-1H-indole-5-carboxylic acid methyl ester (9.4 g, 54%).

A reaction flask equipped with a nitrogen line and a stir bar was charged with 2 (9.4 g, 43.8 mmol) in dichloromethane (100 mL). Di-t-butyl dicarbonate (10.5 g, 48.3 mmol) was added followed by 4-dimethylaminopyridine (0.3 g, 2.2 mmol). The reaction was complete in 3 h. No work up was performed. The reaction mixture was adsorbed onto silica gel and was purified by chromatography on a Combiflash system eluting with an EtOAc/hexane gradient (10-100%) to give to 3-cyanomethyl-indole-1,5-dicarboxylic acid 1-tert-butyl ester 5-methyl ester (10 g, 72%)

A reaction flask equipped with a nitrogen line and a stir bar was charged with 60% sodium hydride in mineral oil (0.5 g, 13.3 mmol). A solution of 3-cyanomethyl-indole-1,5-dicarboxylic acid 1-tert-butyl ester 5-methyl ester (2.0 g, 6.3 mmol) in ethyl ether (40 mL) and DMSO (10 mL) was added, followed by 1,3-dibromopropane (0.7 mL, 6.9 mmol). The reaction mixture was refluxed for 5 h. The reaction mixture was quenched with water and diluted with EtOAc (20 mL). The organic layer was washed with water (2×10 mL). The organic layer was dried over anhydrous magnesium sulfate. The solvent was evaporated and the resulting oil was purified by chromatography on a Combiflash system eluting with an EtOAc/hexane gradient (10-100%) to give 3-(1-cyano-cyclobutyl)-indole-1,5-dicarboxylic acid 1-tert-butyl ester 5-methyl ester (0.6 g, 27%).

A reaction flask equipped with a nitrogen line and a stir bar was charged with 3-(1-cyano-cyclobutyl)-indole-1,5-dicarboxylic acid 1-tert-butyl ester 5-methyl ester (1.2 g, 3.4 mmol) in 50% aqueous trifluoroacetic acid (40 mL). The reaction was complete in 1.2 h. The solvent was evaporated and was diluted with EtOAc. The organic layer was washed with saturated sodium carbonate and was dried with anhydrous magnesium sulfate. The solvent was evaporated to give 3-(1-cyano-cyclobutyl)-1H-indole-5-carboxylic acid methyl ester (0.80 g, 93%).

A Parr hydrogenator flask was charged with a solution of 3-(1-cyano-cyclobutyl)-1H-indole-5-carboxylic acid methyl ester (0.8 g, 3.1 mmol) in absolute EtOH (75 mL). Platinum (1V) oxide (0.5 g) was added. The reaction mixture was shaken under hydrogen at 50 psi for 36 h. The reaction mixture was filtered through diatomaceous earth. The solvent was evaporated and the resulting oil was purified by SCX column in MeOH to give 3-(1-aminomethyl-cyclobutyl)-1H-indole-5-carboxylic acid methyl ester (0.56 g, 69%).

A reaction flask equipped with a nitrogen line and a stir bar was charged with 3-(1-aminomethyl-cyclobutyl)-1H-indole-5-carboxylic acid methyl ester (0.62 g, 2.4 mmol) in toluene (55 mL) and dichloromethane (5 mL). Triethylamine (0.8 mL, 5.7 mmol) was added, followed by triphosgene (0.28 g, 0.96 mmol). The reaction mixture was stirred for 0.5 h. 30% Hydrogen bromide in acetic acid (1 mL) was added. The reaction was stirred for a further 0.5 h. The reaction mixture was diluted with EtOAc and washed with water. The organic layer was dried with anhydrous sodium sulfate. The solvent was evaporated and the resulting oil was purified by chromatography on a Combiflash system eluting with an EtOAc/hexane gradient (1-50%) to give 4,4-spirocyclobutyl-1-oxo-2,3,4,9-tetrahydro-1H-β-carboline-6-carboxylic acid methyl ester (0.2 g, 29%).

A reaction flask equipped with a nitrogen line and a stir bar was charged with a solution of 4,4-spirocyclobutyl-1-oxo-2,3,4,9-tetrahydro-1H-β-carboline-6-carboxylic acid methyl ester (0.33 g, 1.1 mmol) in MeOH (3 mL) and water (3 mL). Sodium hydroxide (0.09 g, 2.3 mmol) was added. The reaction mixture was refluxed for 2 h at 100° C. The solvent was evaporated. The residue dissolved in water and washed with EtOAc. The aqueous layer was acidified with 1M hydrochloric acid to pH 4. The product precipitated. The product was filtered and dried under vacuum at 10° C. to give 4,4-spirocyclobutyl-1-oxo-2,3,4,9-tetrahydro-1H-β-carboline-6-carboxylic acid (0.30 g, 95%).

A microwave vial with a stir bar was charged with a solution of 4,4-spirocyclobutyl-1-oxo-2,3,4,9-tetrahydro-1H-β-carboline-6-carboxylic acid (0.03 g, 0.11 mmol) in DMF (2 mL). 1-(3-Dimethylaminopropyl)-3-ethylcarbodimide hydrochloride (0.05 g, 0.27 mmol) was added, followed by 1-hydroxybenzotriazole hydrate (0.03 g, 0.27 mmol). The reaction mixture was sealed and heated in the microwave for 3 min at 80° C. The reaction mixture was diluted with EtOAc and was washed with water. The aqueous layer was washed with EtOAc. The combined organic layer was dried with anhydrous sodium sulphate. The solvent was evaporated and the resulting oil was purified by chromatography on a Combiflash system eluting with an EtOAc/hexane gradient (50-100%) to give the title compound (0.028 g, 73%). ES-MS m/z 335 [M+H]⁺.

### Example 27

### Synthesis of 3-azidomethyl-1-oxo-2,3,4,9-tetrahydro-1H-β-carboline-6-carboxylic acid

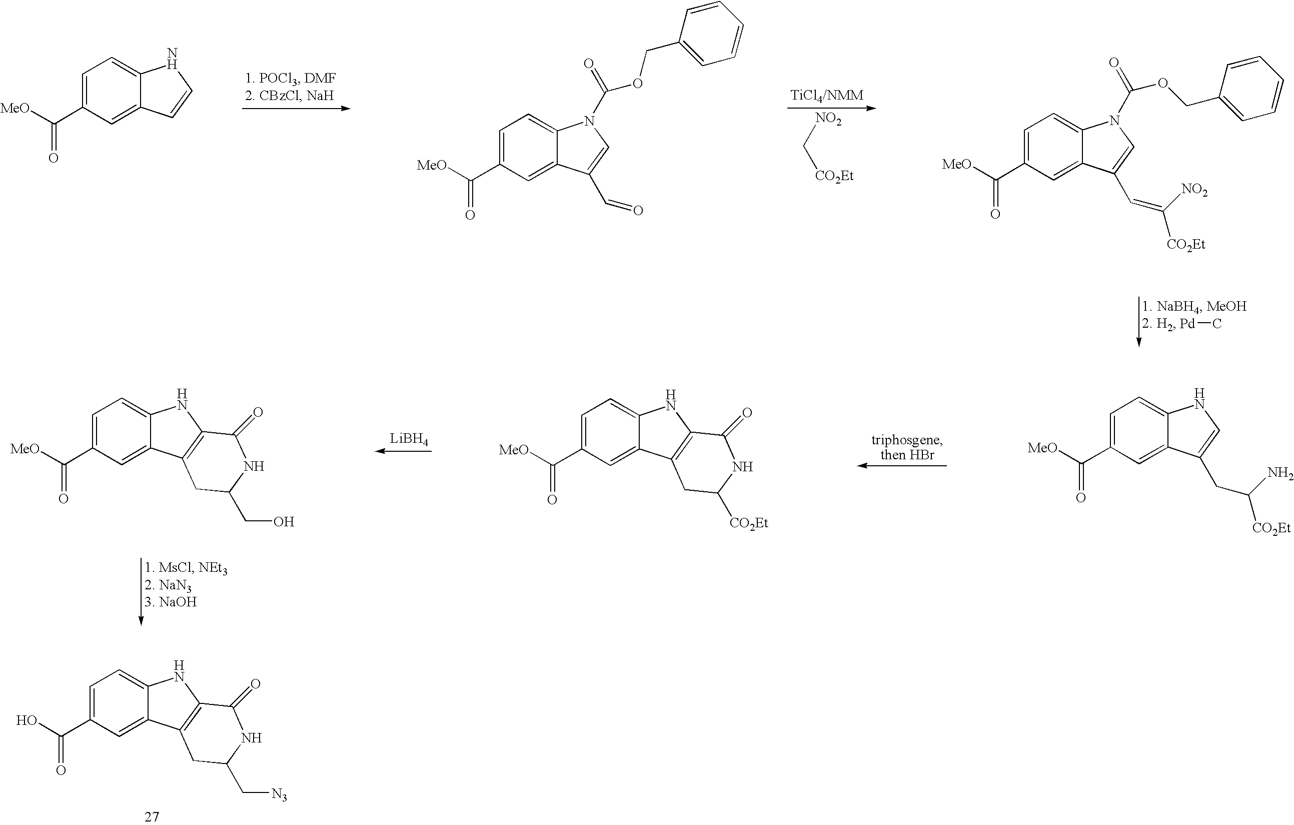

To a solution of 1H-indole-5-carboxylic acid methyl ester (11 g, 62.3 mmol) in 100 mL of DMF at 0° C. was added POCl₃ (7.9 ml, 84.8 mmol) and the reaction mixture was then stirred at ambient temperature for 2 h. The reaction was quenched by adding 350 mL of H₂O and the mixture was heated to reflux for 15 min resulting a brownish suspension. Upon cooling, a lot of precipitates were formed. An additional 300 mL of H₂O were added and the mixture was stirred for 10 min. The solids were collected by filtration and washed with water and hexane and dried in an oven at 60° C. under vacuo overnight to give the desired aldehyde (12.7 g, 99.5% yield) as a light pink solid.

To a solution of the above aldehyde (1.0 g, 4.92 mmol) in 20 mL DMF was added NaH (60% in mineral oil, 220 mg, 5.50 mmol) at 0° C. After 30 min, benzyl chloroformate (0.93 ml, 6.5 mmol) in 5 mL DMF was added and the mixture was stirred at ambient temperature for 12 h. The reaction mixture was poured into 200 mL H₂O, extracted with EtOAc, washed with brine, dried over Na₂SO₄, and concentrated in vacuo. The crude product was recrystallized from EtOH (40 mL) to give 1.4 g (84% yield) of 3-formyl-indole-1,5-dicarboxylic acid 1-benzyl ester 5-methyl ester product as a white solid.

TlCl₄ (2.6 mL, 23.7 mmol) in 100 mL of dry THF was cooled at 0° C. and then a suspension of the above aldehyde (4 g, 11.9 mmol) and ethyl nitroacetate (1.5 ml, 12.6 mmol) in 50 mL of dry THF was added at 0° C. A solution of N-methylmorpholine (5.3 ml, 18.2 mmol) in 40 mL of THF was then added via addition funnel over a period of 60 min. The mixture was stirred at 0° C. to 23° C. for 6 h. The reaction was then quenched with 40 mL of water. The reaction mixture was extracted with EtOAc, washed with brine, dried over Na₂SO₄, filtered, and concentrated to provide 5.71 g of 3-(2-Ethoxycarbonyl-2-nitro-vinyl)-indole-1,5-dicarboxylic acid 1-benzyl ester 5-methyl ester, which was used in next step without further purification.

The above olefin intermediate (2.8 g, 6.19 mmol) was dissolved in MeOH—CH₂Cl₂ (70 ml, 6:1). To this solution was added NaBH₄ (240 mg, 6.32 mmol) in portions, resulting in a yellow suspension. After 30 min, the reaction was quenched by slowly adding 0.2 mL of HOAc giving a clear yellow solution. The reaction mixture was diluted with 150 mL of CH₂Cl₂, washed with water, saturated aqueous NaHCO₃ and brine, then dried over Na₂SO₄. After removal of the solvents, 2.75 g of the partially reduced intermediate was obtained as a yellow solid, which was used in next step without further purification.

The above partially reduced intermediate (2.75 g, 6.05 mmol) was dissolved in HOAc-EtOH-DMA (25 mL, 2:2:1, not totally soluble). To this mixture was added 10% Pd—C (0.8 g) and the mixture was stirred under H₂ (1 atm) for 15 h. LCMS showed the absence of starting material and presence of desired product as the major product along with some partially reduced hydroxylamine product. An additional 400 mg of Pd—C was added and the reaction mixture was stirred under H₂ for 48 h. The solid was removed by filtration and rinsed with MeOH. After removal of most of solvents, the filtrate was diluted with CH₂Cl₂ (200 mL) and 30 mL of saturated aqueous NaHCO₃ were added, and then 30 mL 4 M NaOH were slowly added. The aqueous layer was extracted back with CH₂Cl₂. The combined CH₂Cl₂ extracts were washed with brine and dried over Na₂SO₄. After removal of the solvent, the crude product was purified on a Combiflash column (1-8% MeOH in CH₂Cl₂ with 0.5% NH₄OH) to give 1.0 g (58% yield over 3 steps) of the desired 3-(2-amino-2-ethoxycarbonyl-ethyl)-1H-indole-5-carboxylic acid methyl ester.

The above ester (1.0 g, 3.44 mmol) was dissolved in toluene-CH₂Cl₂ (1:2, 60 mL) and cooled to 0° C. Et₃N (1.2 mL, 8.61 mmol) was added followed by triphosgene (415 mg, 1.4 mmol) in 5 mL of toluene. The mixture was then stirred at ambient temperature for 1 h. A 33% solution of HBr in HOAc (3.0 mL) was then added and the resulting white-yellow suspension was stirred for 2 h. the reaction was then quenched with 15 mL of H₂O giving a clear solution. The reaction mixture was diluted with 100 mL of EtOAc and some product precipitated out. The solid was collected by filtration and the two-layer filtrate was separated. The aqueous layer was extracted with EtOAc. The combined organic layers were washed with water, saturated aqueous NaHCO₃, and brine, then dried over Na₂SO₄ and concentrated in vacuo to give a white solid, which was combined with solids obtained by filtration, and triturated with 10 mL of MeCN to give 0.8 g (73% yield) of 1-oxo-2,3,4,9-tetrahydro-1H-β-carboline-3,6-dicarboxylic acid 3-ethyl ester 6-methyl ester as a white solid.

To suspension of the above diester in 10 mL THF was added LiBH₄ and the reaction was stirred at room temperature for 3 h. TLC showed the alcohol is the major product (70% by LCMS) along with overreduced product diol (30%). The reaction was quenched with water, neutronized with 0.1 mL of HOAc. extracted with EtOAc, washed with NaHCO₃ and brine, dried over Na₂SO₄, and concentrated to give 410 mg of the desired 3-hydroxymethyl-1-oxo-2,3,4,9-tetrahydro-1H-β-carboline-6-carboxylic acid methyl ester, which contained about 10% of the over-reduced diol product (by LCMS and NMR).

To a solution of the above alcohol (330 mg, 1.2 mmol) in 2:1 THF-DMA (10 mL) cooled in ice-bath was added MsCl (0.15 mL, 1.94 mmol) followed by Et₃N (0.28 mL, 2.01 mmol). The mixture was stirred at 0° C. to 23° C. for 2 h. The reaction mixture was then diluted with EtOAc, washed with H₂O and brine, dried over Na₂SO₄ and concentrated in vacuo to give 440 mg of crude mesylate, which was used in next steps without purification.

To a solution of the above mesylate (440 mg, 1.25 mmol) in 8 mL DMSO was added NaN₃ (700 mg, 10.8 mmol) and the mixture was heated at 80° C. for 12 h. The reaction mixture was then diluted with EtOAc, washed with H₂O, brine, dried over Na₂SO₄, concentrated in vacuo to give 336 mg of crude azide ester as a yellow solid, which was used in next step.

To a solution of the above azide ester (336 mg, 1.12 mmol) in 6 mL of THF-MeOH (1:1) was added 2 M aqueous NaOH solution (3.0 mL, 6.0 mmol). The mixture was heated at 7° C. for 3 h. After cooling down, the reaction was diluted with H₂O and extracted with ether. The ether layer was discarded and the aqueous layer was acidified with 2 N HCl to pH 3-4, extracted with EtOAc, washed with H₂O, brine, dried over Na₂SO₄ and concentrated in vacuo to give 250 mg (58% over 3 steps) of the title compound. ES-MS m/z+[M+H]⁺.

### Example 28

### General procedure for amide coupling and hydrogenation of azide

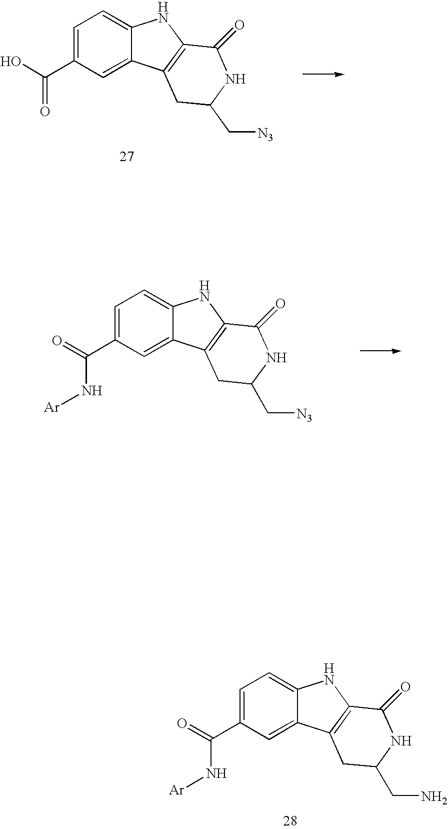

To a solution of 3-azidomethyl-1-oxo-2,3,4,9-tetrahydro-1H-β-carboline-6-carboxylic acid (Example 27) in dry DMF (0.2-0.5 M) was added diisopropylethylamine (2 equiv.), HATU (1.5 equiv.) and HOAt (0.2 equiv.). After 10 min, the desired aniline (1.3-2 equiv.) was added. The reaction mixture was heated at 5° C. for 1-3 days. The reaction mixture was diluted with EtOAc, washed with water, brine, dried over Na₂SO₄ and concentrated in vacuo to give the crude product, which was triturated with MeCN to give the pure amide-azide.

The amide-azide intermediate was dissolved in 2:1 MeOH-DMA. To this solution was added 10% Pd—C (30-50 wt. %) and the mixture was stirred under H₂ (1 atm) for 2-12 h. The reaction mixture was then filtered through a layer of diatomaceous earth, and the solid residue was rinsed with MeOH. The filtrate was concentrated and the remaining DMA was removed by blowing N₂. The residue was triturated from MeCN or MeOH to give the desired aminomethyl product.

The following compounds were synthesized by the above general procedure:
3-Aminomethyl-1-oxo-2,3,4,9-tetrahydro-1H-β-carboline-6-carboxylic acid pyridin-3-ylamide; ES-MS m/z 336 [M+H]⁺. 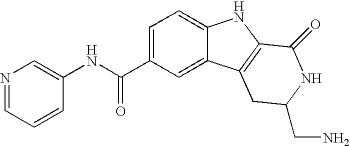
3-Aminomethyl-1-oxo-2,3,4,9-tetrahydro-1H-β-carboline-6-carboxylic acid quinolin-3-ylamide; ES-MS m/z 428.2 [M+H]⁺. 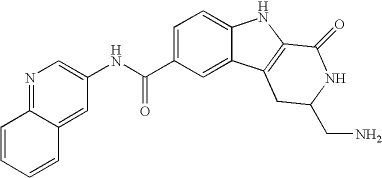
3-Aminomethyl-1-oxo-2,3,4,9-tetrahydro-1H-β-carboline-6-carboxylic acid pyrazinylamide; ES-MS m/z 337.1 [M+H]⁺. 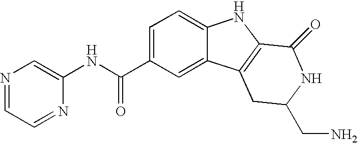
3-Aminomethyl-1-oxo-2,3,4,9-tetrahydro-1H-β-carboline-6-carboxylic acid thiazol-2-yl amide; ES-MS m/z 341.4 [M+H]⁺.

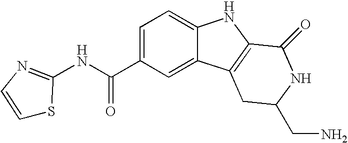

### Example 29

### Synthesis of 3-hydroxymethyl-1-oxo-2,3,4,9-tetrahydro-1H-β-carboline-6-carboxylic acid pyridin-3-ylamide

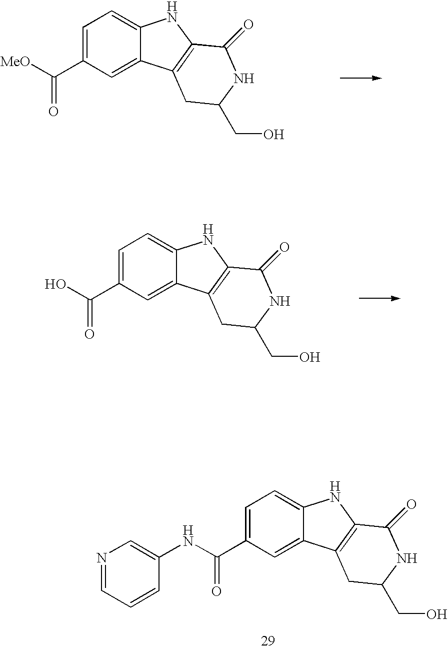 [0393] 3-Hydroxymethyl-1-oxo-2,3,4,9-tetrahydro-1H-β-carboline-6-carboxylic acid methyl ester (intermediate from Example 27) (140 mg, 0.51 mmol) was suspended in 1:1 MeOH-THF (5 mL). To this suspension was added 1 M aqueous NaOH (2.5 mL, 2.5 mmol). The mixture was heated at 70° C. for 2 h. After cooling, the reaction was diluted with H₂O, acidified with 2 N HCl resulting in formation of a yellow precipitate, which was collected by filtration and washed with water, dried in an oven at 65° C. in vacuo to give 100 mg (75% yield) of the desired carboxylic acid.

To a solution of the above carboxylic acid (100 mg, 0.38 mmol) in dry DMF (2 ml) was added diisopropylethylamine (0.14 mL, 0.80 mmol), HATU (230 mg, 0.60 mmol) and HOAt (10 mg, 0.07 mmol). After 10 min, 3-aminopyridine (60 mg, 0.64 mmol) was added. The reaction mixture was heated at 55° C. for 10 h and then ambient temperature overnight. The solvent was removed in vacuo and the residue was purified on a Combiflash column (eluting with 5-10% MeOH in CH₂Cl₂) to give 70 mg of the crude product with ˜20% impurity by NMR. Further purification by preparative TLC gave 50 mg (39% yield) of product. 35 mg of the product from preparative TLC was triturated with MeCN to give 25 mg of pure title compound. ES-MS m/z 336.4 [M+H]⁺.

### Example 30

### Synthesis of 3-Aminomethyl-1-oxo-2,3,4,9-tetrahydro-1H-β-carboline-6-carboxylic acid [4-(2-dimethylamino-ethylcarbamoyl)-thiazol-2-yl]-amide

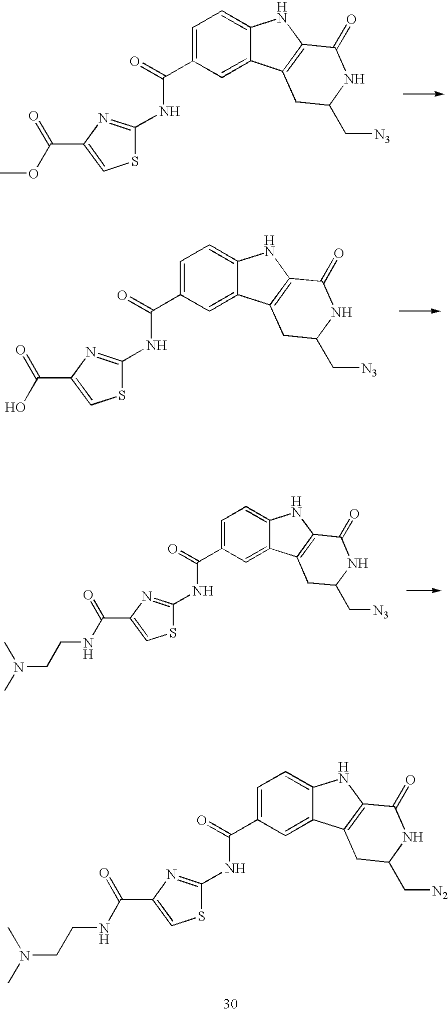

2-[(3-Azidomethyl-1-oxo-2,3,4,9-tetrahydro-1H-β-carboline-6-carbonyl)-amino]-thiazole-4-carboxylic acid methyl ester (230 mg, 0.54 mmol) was suspended in 1:1 THF-MeOH (5 mL), and 2 M aqueous NaOH (1 mL, 2.0 mmol) was added. The resulting clear orange solution was heated at 7° C. for 5 h. The organic solvents were removed in vacuo, the residue was diluted with 15 mL of water and acidified with 2 N HCl to pH 1. The precipitates were collected by filtration, washed with water, and dried in an oven at 6° C. in vacuo to give 170 mg (76% yield) of the desired carboxylic acid.

A mixture of the above carboxylic acid (100 mg, 0.24 mmol), EDC (60 mg, 0.31 mmol), HOBt (45 mg, 0.33 mmol), N,N-dimethylethylenediamine (0.033 ml, 0.31 mmol), diisopropylethylamine (0.1 ml, 0.57 mmol) and DMAP (6 mg, 0.05 mmol) was stirred at ambient temperature for 5 h. DMF was removed by blowing N₂. The residue was dissolved in 2 mL of MeOH, diluted with 50 mL of EtOAc and washed with water. The aqueous layer was re-extracted with EtOAc for 3 times. The combined extracts were washed with brine and dried over Na₂SO₄. After removal of the solvents, the residue was triturated with 3 mL of MeCN to give 80 mg (68% yield) of the desired amide.

By using the general procedure for the hydrogenation of azides described in Example 28, the title compound was prepared in 68% yield. ES-MS m/z 455.8 [M+H]⁺.

### Example 31

### Synthesis of {6-[4-(2-Dimethylamino-ethylcarbamoyl)-thiazol-2-ylcarbamoyl]-1-oxo-2,3,4,9-tetrahydro-1H-β-carbolin-3-ylmethyl}-carbamic acid ethyl ester

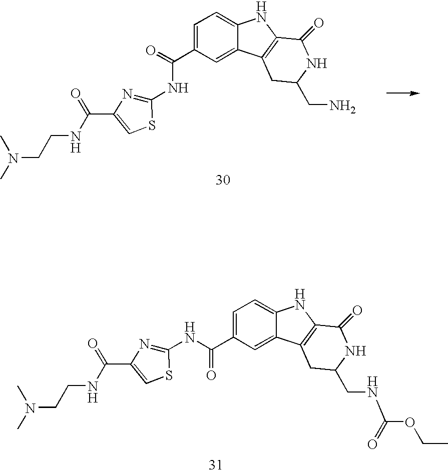

To a solution of 3-aminomethyl-1-oxo-2,3,4,9-tetrahydro-1H-β-carboline-6-carboxylic acid [4-(2-dimethylamino-ethylcarbamoyl)-thiazol-2-yl]-amide (24 mg, 53 μmol) in 0.6 mL of DMF was added Et₃N (10 μl, 72 μmol) followed by ethyl chloroformate (6 μl, 63 μmol). The mixture was stirred at ambient temperature for 30 min. The reaction mixture was then diluted with EtOAc, washed with saturated aqueous NaHCO₃, water, brine, dried over Na₂SO₄ and concentrated in vacuo to give 27 mg of crude product, which was triturated with MeOH to give 15 mg (54% yield) of the title compound as a white solid. ES-MS m/z 527.9 [M+H]⁺.

The following compounds were prepared by the procedure described in the above Example, replacing ethyl chloroformate with the appropriate anhydride
[1-Oxo-6-(pyridin-3-ylcarbamoyl)-2,3,4,9-tetrahydro-1H-β-carbolin-3-yl methyl]-carbamic acid tert-butyl ester 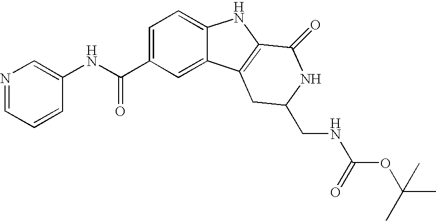
   was prepared in 12% yield using Boc anhydride. ES-MS m/z 435.49 [M+H]⁺.
3-(Acetylamino-methyl)-1-oxo-2,3,4,9-tetrahydro-1H-β-carboline-6-carboxylic acid quinolin-3-ylamide 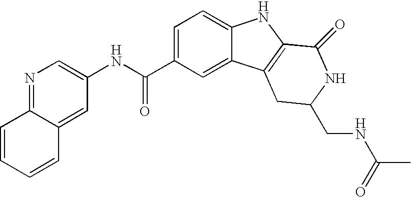
   was prepared in 64% yield using acetic anhydride. ES-MS m/z 428.2 [M+H]⁺.

### Example 32

### Synthesis of 3-(2-cyano-ethyl)-1-oxo-2,3,4,9-tetrahydro-1H-β-carboline-6-carboxylic acid pyridin-3-ylamide

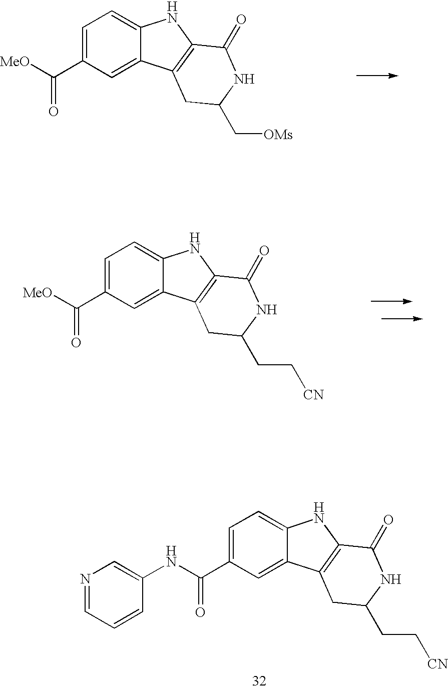

A mixture of the mesylate ester of 3-hydroxymethyl-1-oxo-2,3,4,9-tetrahydro-1H-β-carboline-6-carboxylic acid methyl ester (200 mg, 0.57 mmol) and NaCN (170 mg, 3.47 mmol) in 3 mL of MDF was stirred at ambient temperature for 12 h and then heated at 6° C. for an additional 10 h. The reaction mixture was diluted with EtOAc, washed with water, brine, dried over Na₂SO₄ and concentrated in vacuo to give 105 mg (65% yield) of the crude nitrile intermediate, which was used in next step without purification.

The above nitrile intermediate (80 mg, 0.28 mmol) was suspended in 1:1 MeOH-THF (3 mL) and 2 M aqueous NaOH (0.4 mL, 0.8 mmol) was added. The mixture was stirred at ambient temperature for 2 days, and then heated at 35° C. for 12 h. The reaction was not completed so an additional 4 M NaOH (0.1 mL, 0.4 mmol) was added and the mixture as heated at 45° C. for another 3 h. The reaction mixture was diluted with water, acidified with 2 N HCl and extracted with EtOAc. The extracts were washed with brine, dried over Na₂SO₄ and concentrated in vacuo to give 69 mg (90% yield) of crude carboxylic acid which was used in next step without purification.

By using the general coupling procedure, the title compound was prepared in 46% from the corresponding acid. ES-MS m/z 359.4 [M+H]⁺.

### Example 33

### Synthesis of 1-oxo-1,2,3,4,5,10-hexahydro-azepino[3,4-b]indole-7-carboxylic acid pyridin-3-ylamide as the hydrochloride salt

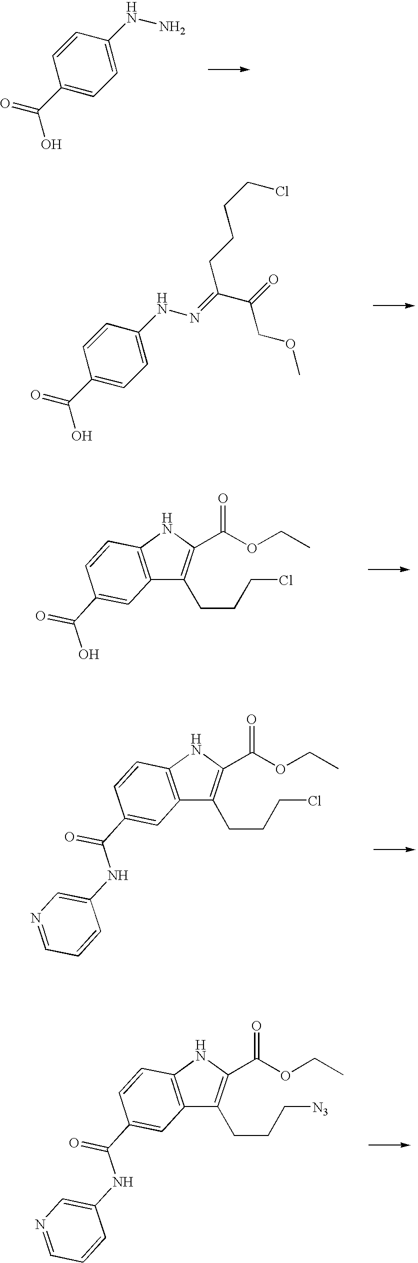

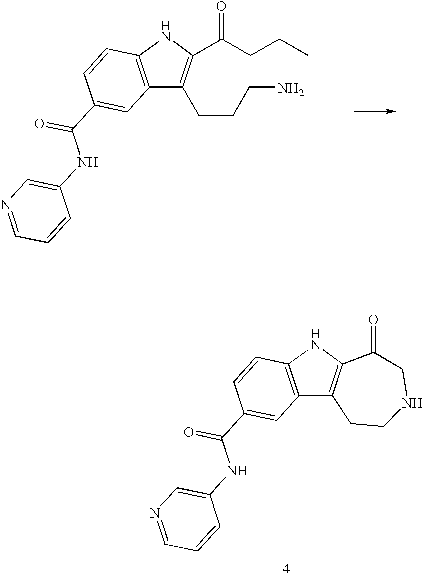

-Hydrazinobenzoic acid (650 mg, 4.27 mmol) and 6-chloro-2-oxohexanoic acid ethyl ester (800 mg, 4.15 mmol) were dissolved in 80 mL MeOH. 0.4 mL acetic acid was added and the mixture was refluxed for 1.5 h. The solvent was evaporated to provide 4-{N′-[5-chloro-1-ethoxycarbonyl-pent-(E)-ylidene]-hydrazino}-benzoic acid, which was used directly without further purification.

4-{N′-[5-Chloro-1-ethoxycarbonyl-pent-(E)-ylidene]-hydrazino}-benzoic acid (1.35 g, 4.15 mmol) was dissolved in 20 mL acetic acid. Boron trifluoride etherate (3 mL, 21.2 mmol) was added. The mixture was heated at 100° C. for 1 h, after which time an equivalent portion of the boron trifluoride etherate was added and the mixture was heated at 100° C. for an additional 1 h. The mixture was cooled to room temperature and the crystallized product was collected by filtration. The filtrate was washed with water providing pure 3-(2-chloro-ethyl)-1H-indole-2,5-dicarboxylic acid 2-ethyl ester. The mother liquors were extracted with EtOAc, dried and concentrated to give additional product, in total providing a near quantitative yield.

3-(2-Chloro-ethyl)-1H-indole-2,5-dicarboxylic acid 2-ethyl ester (180 mg, 0.58 mmol) was dissolved in 4 mL DMF in a reaction tube. The tube was flushed with N₂, sealed with a cap, and cooled to 0° C. The cap was removed and EDC (134 mg, 0.7 mmol) and HOBT (94.6 mg, 0.7 mmol) were added quickly as solids, and the cap was replaced. The reaction mixture was stirred at 0° C. for 1 h and ambient temperature for 1 h, after which time it was cooled to 0° C., the cap was removed, and 3-aminopyridine (112 mg, 1.19 mmol) was added as a solid. The tube was again flushed with N₂ and sealed with a cap. The ice was removed and the reaction mixture was allowed to stir at rt for 120 h. The reaction mixture was poured into water, and the resulting mixture was extracted 3× with ethyl acetate. The combined organic extracts were washed with a saturated aqueous solution of NaHCO₃ and brine, and evaporated. The resulting product was triturated with dichloromethane and filtered to provide 200 mg of pure 3-(3-chloro-propyl)-5-(pyridin-3-ylcarbamoyl)-1H-indole-2-carboxylic acid ethyl ester.

3-(3-Chloro-propyl)-5-(pyridin-3-ylcarbamoyl)-1H-indole-2-carboxylic acid ethyl ester (170 mg, 0.44 mmol) and sodium azide (65 mg, 1 mmol) were dissolved in 2 mL DMSO and the mixture was heated at 10° C. for 1.5 h, after which time it was cooled and poured into water. The resulting mixture was extracted 2× with ethyl acetate, and the combined extracts were washed with water, then with brine, dried and evaporated to provide 175 mg pure 3-(3-azido-propyl)-5-(pyridin-3-ylcarbamoyl)-1H-indole-2-carboxylic acid ethyl ester.

3-(3-Azido-propyl)-5-(pyridin-3-ylcarbamoyl)-1H-indole-2-carboxylic acid ethyl ester (175 mg, 0.44 mmol) and 5 mg 10% Pd/C were combined in a reaction tube with 2 mL methanol, and the tube was flushed with N₂ and evacuated, then filled with H₂. The tube was again evacuated then filled with H₂ twice more. The resulting mixture was stirred at ambient temperature under a balloon of H₂ for 1 h, after which time the balloon was removed and the catalyst was filtered. The solvents were then evaporated and the resulting product was triturated with Et₂O and filtered to provide 123 mg 3-(3-amino-propyl)-5-(pyridin-3-ylcarbamoyl)-1H-indole-2-carboxylic acid ethyl ester.

3-(3-Amino-propyl)-5-(pyridin-3-ylcarbamoyl)-1H-indole-2-carboxylic acid ethyl ester (91 mg, 0.24 mmol) and AlMe₃ (2M in hexanes, 0.6 mL, 1.2 mmol) were combined in 3 mL THF and stirred at rt for 4 d. The reaction was quenched with a few drops of MeOH, followed by a solution of NH₄OH. The resulting mixture was extracted with ethyl acetate (the two phases were allowed to stir for 5 days before separating the organic layer). The aqueous was then extracted with a second portion of ethyl acetate. The organic extracts were combined, washed with brine, dried and evaporated. The product was purified by flash chromatography on silica gel using 0-15% of a solution of 9:1 methanol:NH₄OH in DCM as eluant. The product was further purified by treatment with 1 N HCl. After stirring for 1 hour, the purified product was isolated by filtration and dried to yield 19 mg of 1-oxo-1,2,3,4,5,10-hexahydro-azepino[3,4-b]indole-7-carboxylic acid pyridin-3-ylamide as the hydrochloride salt; ES-MS m/z 320.4 [M+H]⁺.

Procedure for the Identification of MAPKAP-k2 Inhibitors

The protein reagents required for the phosphoryl transfer reaction catalyzed by MAPKAP-k2 include 1 nM MAPKAP-k2 (1-400) and 500 nM biotinylated LSP1 (179-339). The MAPKAP-k2 (1-400) splice variant used in the reaction is expressed as an amino-terminal glutathione transferase fusion protein in insect cells, purified by glutathione affinity chromatography and activated with murine p38α (Lukas et al., (2004) Biochemistry 43, 9950-9960). The biotinylated LSP1 (179-339) is prepared from an amino-terminal GST fusion of the carboxy-terminal portion of lymphocyte specific protein 1 (LSP1 179-339), expressed in E. coli and purified by glutathione affinity chromatography (Lukas et al., (2004) Biochemistry 43, 9950-9960). LSP1 (179-339) is covalently modified with iodoacetyl biotin (Pierce Chemicals). The phosphoryl transfer reaction (30 min) is performed in Reacti-Bind NeutrAvidin Coated plates (Pierce Chemicals) in buffer containing 50 mM HEPES (pH 7.6), 50 mM KCl, 10 mM MgCl₂, 100 μM Na₃VO₄, 0.01% CHAPS, 1 mM DTT, 10 μg/mL bovine serum albumin, 2 μM ATP, 0-30 μM compound and 1-2% DMSO (v/v).

The protein reagent required for detection of MAPKAP-k2 (1-400) dependent phosphorylation of biotinylated LSP1 (179-339) and inhibitors of MAPKAP-k2 (1-400) catalysis is Eu³⁺ chelated anti-phospho-LSP1 IgG1 monoclonal antibody. The anti-phospho-LSP1 IgG, monoclonal antibody is raised against the following amino acid sequence: CRTPKLARQA(phospho-S)IELPSM (Anaspec) conjugated to KLH antigen from Pierce Chemicals. The antibody is covalently modified with Eu N1 ITC Chelate from Perkin Elmer Life Sciences. The detection of MAPKAP-k2 (1-400) dependent phosphorylation of LSP1 (179-339) and inhibitors of MAPKAP-k2 (1-400) catalysis is performed by (1) washing the Reacti-Bind NeutrAvidin Coated plates with Delfia Wash Buffer (Perkin Elmer Life Sciences), (2) adding the Eu³⁺ chelated anti-phospho-LSP1 IgG1 monoclonal antibody to the Reacti-Bind NeutrAvidin Coated plates (1 hr), (3) washing the Reacti-Bind NeutrAvidin Coated plates with Delfia Wash Buffer, (4) adding Delfia Enhancement Solution (15 min) from Perkin Elmer Life Sciences, and (5) reading time resolved fluorescence with an excitation maximum of 360 nm and an emission maximum of 620 nm on an LJL Biosystems Analyst instrument.

Inhibition of TNF Production in THP Cells

The inhibition of cytokine production can be observed by measuring inhibition of TNFα in lipopolysaccharide stimulated THP cells (for example, see W. Prichett et al., 1995, J. Inflammation, 45, 97). All cells and reagents were diluted in RPMI 1640 with phenol red and L-glutamine, supplemented with additional L-glutamine (total: 4 mM), penicillin and streptomycin (50 units/ml each) and fetal bovine serum (FBS, 3%) (GIBCO, all conc. final). Assay was performed under sterile conditions; only test compound preparation was nonsterile. Initial stock solutions were made in DMSO followed by dilution into RPMI 1640 2-fold higher than the desired final assay concentration. Confluent THP.1 cells (2×10⁶ cells/ml, final conc.; American Type Culture Company, Rockville, Md.) were added to 96 well polypropylene round bottomed culture plates (Costar 3790; sterile) containing 125 μl test compound (2 fold concentrated) or DMSO vehicle (controls, blanks). DMSO concentration did not exceed 0.2% final. Cell mixture was allowed to preincubate for 30 min, 37° C., 5% CO₂ prior to stimulation with lipopolysaccharide (LPS; 1 μg/ml final; Siga L-2630, from E. coli serotype 0111.B4; stored as 1 mg/ml stock in endotoxin screened distilled H₂O at −80° C.). Blanks (unstimulated) received H₂O vehicle; final incubation volume was 250 μl. Overnight incubation (18-24 hr) proceeded as described above. Assay was terminated by centrifuging plates 5 min, room temperature, 1600 rpm (400×g); supernatants were transferred to clean 96 well plates and stored −80° C. until analyzed for human TNFα by a commercially available ELISA kit (Biosource #KHC3015, Camarillo, Calif.). Data was analyzed by non-linear regression (Hill equation) to generate a dose response curve using SAS Software System (SAS institute, Inc., Cary, N.C.). The calculated IC₅₀ value is the concentration of the test compound that caused a 50% decrease in the maximal TNFα production.

Lipopolysaccharide (LPS)-Induced TNF-Alpha Production:

B10.RIII mice were obtained from Jackson laboratories, LPS from Sigma Chemical Co. (# L-2880), D-+-galactosamine from Sigma chemical Co. (# G-0500) and Aerrane (isoflurane, USP) from Baxter Pharmaceuticals, NDC 10019-773-40. Animals were weighed and their tails were marked. Mice were anesthetized with isoflurane and their tails were warmed with gauze dipped in hot water prior to challenge. They were challenged with 200 ng of LPS/1 mg galactosamine in 200 uL per mouse delivered intravenously (i.v.) into the tail vein. 1 hour after challenge, the mice were anesthetized with isoflurane and were bled by cardiac punction. Approximately 100 uL were collected. The blood was dispensed into eppendorf tubes treated with EDTA and shaken. This procedure was repeated for all animals. The blood was centrifuged for ˜5 minutes at 14,000 rpm in an eppendorf microfuge. The plasma was collected, put into labeled eppendorf tubes and frozen at −20° C. Plasma samples were then assayed for the presence of TNF-alpha using a mouse TNF-alpha ELISA duoset (DY410) kit purchased form R&D systems conducted as per the protocol supplied. Plasma samples are diluted to allow samples to fall on the linear portion of the standard response curve.

Methods of Using the Compounds of the Present Invention

In accordance with the invention, there are provided novel methods of using the compounds of the present invention. The compounds disclosed therein effectively block inflammatory cytokine production from cells. The inhibition of cytokine production is an attractive means for preventing and treating a variety of cytokine mediated diseases or conditions associated with excess cytokine production, e.g., diseases and pathological conditions involving inflammation. Thus, the compounds are useful for the treatment of diseases and conditions as described in the Background section, including the following conditions and diseases: osteoarthritis, atherosclerosis, contact dermatitis, bone resorption diseases, reperfusion injury, asthma, multiple sclerosis, Guillain-Barre syndrome, Crohn's disease, ulcerative colitis, psoriasis, graft versus host disease, systemic lupus erythematosus and insulin-dependent diabetes mellitus, rheumatoid arthritis, toxic shock syndrome, Alzheimer's disease, diabetes, inflammatory bowel diseases, acute and chronic pain as well as symptoms of inflammation and cardiovascular disease, stroke, myocardial infarction, alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, syndromes associated with hemodialysis, leukopherisis, granulocyte transfusion associated syndromes, and necrotizing entrerocolitis, complications including restenosis following percutaneous transluminal coronary angioplasty, traumatic arthritis, sepsis, chronic obstructive pulmonary disease and congestive heart failure. The compounds of the invention may also be useful for anticoagulant or fibrinolytic therapy (and the diseases or conditions related to such therapy).

The compounds of the invention will be useful for treating oncological diseases and other cytokine mediated diseases and conditions related to p38 and MK2 as known in the art. These diseases include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma and mesothelioma.

Examples of brain cancers include, but are not limited to brain stem, optic and hypophtalmic glioma, cerebella and cerebral astrocytoma, medulloblastoma, ependymoma, as well as pituitary, neuroectodermal and pineal tumor.

Examples of peripheral nervous system tumors include, but are not limited to neuroblastoma, ganglioneuroblastoma, and peripheral nerve sheath tumors.

Examples of tumors of the endocrine and exocrine system include, but are not limited to thyroid carcinoma, adrenocortical carcinoma, pheochromocytoma, and carcinoid tumors.

Tumors of the male reproductive organs include, but are not limited to prostate and testicular cancer.

Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallblader, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), hepatoblastoma, cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, Hodgkins lymphoma, cutaneous T-cell lymphoma, and lymphoma of the central nervous system.

Sarcomas include, but are not limited to sarcoma of the soft tissue, osteosarcoma, Ewings sarcoma, malignant fibrous histiocytoma, lymphosarcoma, angiosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

Plasma cell dyscrasias include, but are not limited to multiple myeloma, and Waldenstrom's macroglobulinemia.

These disorders have been well characterized in man, but also exist with a similar etiology in other mammals, and can be treated by pharmaceutical compositions of the present invention.

For therapeutic use, the compounds may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. The above described compounds may be physically combined with the conventional therapeutics or other adjuvants into a single pharmaceutical composition. Reference is this regard may be made to Cappola et al.: U.S. Pat. No. 6,565,880, PCT/US 01/21860 and U.S. application Ser. No. 10/214,782, each incorporated by reference herein in their entirety. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds may be administered separately (either serially or in parallel). Separate dosing allows for greater flexibility in the dosing regime.

As mentioned above, dosage forms of the compounds described herein include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. Reference in this regard may also be made to US publication No. US 2003-0118575 A1. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

We claim:

1. A compound of formula (I) 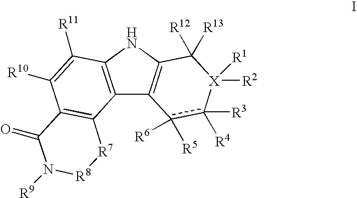
wherein:
X is C or N
R¹ is hydrogen, hydroxy, carbamoyl, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆alkylthio C₁₋₆alkyl, wherein the sulfur atom, when present, is optionally oxidized to a sulfoxide or sulfone;
R² is optionally present and, if present, is hydrogen, hydroxy, ureido, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆ alkylthio, wherein the sulfur atom, when present, is optionally oxidized to a sulfoxide or sulfone;
R³ is hydrogen, amino, C₁₋₆ alkyl, C₂₋₆ alkenyl, amino C₁₋₆ alkyl, hydroxyC₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbamoyl, CO₂C₁₋₆alkyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x} wherein R^{x} can be chosen from:
hydrogen, amino, C₁₋₆alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, —CO₂C₁₋₆alkyl or C₂₋₆ alkylthio wherein the sulfur atom is oxidized to a sulfoxide or sulfone;
R⁴ is optionally present and, when present, is hydrogen, amino, C₁₋₆ alkyl, C₂₋₆ alkenyl, amino C₁₋₆ alkyl, hydroxy C₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbonyl, carbamoyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x}, and wherein R^{x} can be chosen from:
amino, C₁₋₆ alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, or C₂₋₆ alkylthio wherein the sulfur atom is oxidized to a sulfoxide or sulfone;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, C₃₋₇ cycloalkyl, heterocyclyl, heterocyclyl C₁₋₆alkyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted by R^{x}, which is defined hereinabove, or ═N—OH, ═N—NHheteroaryl;
R⁴ and R⁵, along with their substituents, may optionally be taken together to form a C₃₋₈ cycloalkyl optionally substituted with amino, C₁₋₃ alkyl, halogen, or hydroxy;
R⁶ is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted with single or multiple R^{x}, which is defined hereinabove, or
R⁵ and R⁶, along with their substituents, may optionally be taken together to form a C₃₋₇ cycloalkyl that may be optionally substituted with single or multiple R^{x}, which is defined hereinabove;
R⁷ is selected from hydrogen, C₁₋₆ alkyl, C₃₋₇ cycloalkyl, C₁₋₆ alkoxy, C1-6alkoxyC1-6 alkyl, hydroxy, hydroxy C₁₋₆ alkyl or amino optionally mono- or di-substituted by C₁₋₆ alkyl, aryl or aryl C1-6 alkyl, or R⁷ may be halo, haloalkyl, alkoxy, carboxyl, carbamoyl, nitro, dialkylamino, benzyloxy, hydrazinocarbonyl, alkoxycarbonyl, alkoxycarbonylheterocyclcarbonyl, or hydroxy; wherein each alkyl, cycloalkyl, alkenyl, aryl or heteroaryl is optionally substituted with single or multiple R^{x}, which is defined hereinabove,
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₁₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl or C₂₋₆ alkylthio, wherein the sulfur atom is optionally oxidized to a sulfoxide or sulfone; wherein each aryl, heterocycle or heteroaryl may be singly or multiply substituted with R^{y}, wherein R^{y} may be chosen from:
halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, C₂₋₆ alkylthio wherein the sulfur atom is optionally oxidized to a sulfoxide or sulfone, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH, —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where Re is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino; or
R⁸ and R⁹ can be taken together to form a cycloalkyl or heteraryl ring, with the proviso that it can not be morpholine;
R¹⁰ and R¹¹ are independently selected from hydrogen, C₁₋₆ alkoxy, hydroxy, halo, C₁₋₆ alkyl, C₃₋₇ cycloalkyl;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆ alkyl,
R¹³ is optionally present and, if present, is selected from from hydrogen, C₁₋₆ alkyl, or a halo group
or the pharmaceutically acceptable salts thereof.

2. The compound according to claim 1 wherein:
X is C or N;
R¹ is hydrogen;
R² is optionally present and, if present, is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is hydrogen or C₁₋₆alkyl;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
R⁶ is hydrogen or C₁₋₆ alkyl, optionally partially or fully halogenated, or
R⁵ and R⁶, may optionally be taken together to form a C₃₋₇ cycloalkyl;
R⁷ is hydrogen, hydroxyl or C₁₋₆ alkoxy;
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, C₁₋₆ alkyl,
piperidinyl, phenyl and heteroaryl, wherein each phenyl, piperidinyl or heteroaryl may be singly or multiply substituted with R^{y}, wherein R^{y} may be chosen from:
halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
R¹³ is optionally present and, if present, is selected from from hydrogen, C₁₋₆ alkyl, or a halo group.

3. The compound according to claim 2 wherein:
X is C or N;
R¹ is hydrogen;
R² is optionally present and, if present, is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆alkoxy, C₂₋₆alkenyloxy, C₂₋₆alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is hydrogen or C₁₋₆alkyl;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
R⁶ is hydrogen or C₁₋₆ alkyl, optionally partially or fully halogenated, or
R⁵ and R⁶, may optionally be taken together to form a C₃₋₇ cycloalkyl;
R⁷ is hydrogen, hydroxyl, or C₁₋₆ alkoxy;
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl and pyrrolyl, thiophene, benzothiophene, furanyl; benzofuran wherein each phenyl, piperidinyl or heteroaryl may be singly or multiply substituted with R^{y}, wherein R^{y} may be chosen from:
halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
R¹³ is optionally present and, if present, is selected from from hydrogen, C₁₋₆ alkyl, or a halo group.

4. The compound according to claim 3 wherein:
X is N;
R¹ is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is selected from hydrogen, or C₁₋₆alkyl;
R⁵ is optionally present and, when present, is hydrogen, C₁₋₆ alkyl or CF₃;
R⁶ is hydrogen, C₁₋₆ alkyl or CF₃; or
R⁵ and R⁶, may optionally be taken together to form a C₃₋₇ cycloalkyl;
R⁷ is hydrogen;
R⁸ is hydrogen;
R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran, wherein each phenyl, piperidinyl or heteroaryl may be singly or multiply substituted with R^{y}, wherein R^{y} may be chosen from:
halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂ or —C(O)N(R^{a})(R^{b}) where R^{a} and R^{b} are independently selected from
H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, and —C(O)NH—N═C—R^{e}, where R^{e} is phenyl, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen; and
R¹² is oxo.

5. A compound of formula (II) 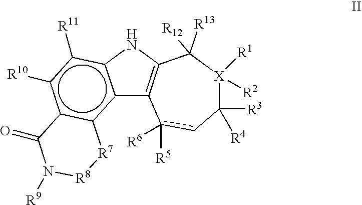
wherein:
X is C or N
R¹ is hydrogen, hydroxy, carbamoyl, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆alkylthio C₁₋₆alkyl, wherein the sulfur atom, when present, is optionally oxidized to a sulfoxide or sulfone;
R² is optionally present and, if present, is hydrogen, hydroxy, ureido, C₁₋₆ alkyl, C₂₋₆ alkenyl, C₂₋₆ alkynyl, C₁₋₆ alkoxy, C₂₋₆ alkenyloxy, C₂₋₆ alkynyloxy, C₃₋₇ cycloalkyl, or C₁₋₆ alkylthio, wherein the sulfur atom, when present, is optionally oxidized to a sulfoxide or sulfone;
R³ is hydrogen, amino, C₁₋₆ alkyl, C₂₋₆ alkenyl, amino C₁₋₆ alkyl, hydroxyC₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbamoyl, CO₂C₁₋₆alkyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x} wherein R^{x} can be chosen from:
hydrogen, amino, C₁₋₆alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, —CO₂C₁₋₆alkyl or C₂₋₆ alkylthio wherein the sulfur atom is oxidized to a sulfoxide or sulfone;
R⁴ is optionally present and, when present, is hydrogen, amino, C₁₋₆ alkyl, C₂₋₆ alkenyl, amino C₁₋₆ alkyl, hydroxy C₁₋₆ alkyl, cyano C₁₋₆ alkyl, C₁₋₆ alkynyl, C₁₋₆ alkoxy, C₂₋₆ alkenyloxy, C₂₋₆ alkynyloxy, hydroxy, C₃₋₇ cycloalkyl, carbonyl, carbamoyl, or C₁₋₆ alkylthio wherein the sulfur atom is optionally oxidized to a sulfoxide or sulfone; wherein each alkyl, amino, cycloalkyl, alkenyl, alkoxy, alkenyloxy, alkynyloxy, carbonyl, carbamoyl, and alkylthio group is optionally substituted by R^{x}, and wherein R^{x} can be chosen from:
amino, C₁₋₆ alkyl, C₂₋₆ alkenyl, amino C₁₋₆ alkyl, hydroxyC₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl-C₁₋₆ alkyl, aryl-C₁₋₁₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, carbonyl, carbamoyl, ureido, or C₂₋₆ alkylthio wherein the sulfur atom is oxidized to a sulfoxide or sulfone;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, C₃₋₇ cycloalkyl, heterocyclyl, heterocyclylC₁₋₆alkyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted by R^{x}, which is defined hereinabove, or ═N—OH, ═N—NHheteroaryl;
R⁴ and R⁵, along with their substituents, may optionally be taken together to form a C₃₋₈ cycloalkyl optionally substituted with amino, C₁₋₃ alkyl, halogen, or hydroxy;
R⁶ is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, or C₁₋₆ alkylthio, wherein the sulfur atom is oxidized to a sulfoxide or sulfone, and wherein each alkyl, cycloalkyl, alkenyl, aryl is optionally substituted with single or multiple R^{x}, which is defined hereinabove, or
R⁵ and R⁶, along with their substituents, may optionally be taken together to form a C₃₋₇ cycloalkyl that may be optionally substituted with single or multiple R^{x}, which is defined hereinabove;
R⁷ is selected from hydrogen, C₁₋₆ alkyl, C₃₋₇ cycloalkyl, C₁₋₆ alkoxy, C1-6alkoxyC1-6 alkyl, hydroxy, hydroxy C₁₋₆ alkyl or amino optionally mono- or di-substituted by C₁₋₆ alkyl, aryl or aryl C1-6 alkyl, or R⁷ may be halo, haloalkyl, alkoxy, carboxyl, carbamoyl, nitro, dialkylamino, benzyloxy, hydrazinocarbonyl, alkoxycarbonyl, alkoxycarbonylheterocyclcarbonyl, or hydroxy; wherein each alkyl, cycloalkyl, alkenyl, aryl or heteroaryl is optionally substituted with single or multiple R^{x}, which is defined hereinabove,
R³ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₁₆
haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl or C₂₋₆ alkylthio, wherein the sulfur atom is optionally oxidized to a sulfoxide or sulfone; wherein each aryl, heterocycle or heteroaryl may be singly or multiply substituted with R^{y}, wherein R^{y} may be chosen from:
halogen, C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, heterocyclyl, aryl, aryloxy, acyl, heteroaryl, C₃₋₇ cycloalkyl —C₁₋₆ alkyl, aryl-C₁₋₆ alkyl, aryl-C₁₋₆ haloalkyl, heterocyclyl-C₁₋₆ alkyl, heteroaryl-C₁₋₆ alkyl, C₃₋₇ cycloalkyl-C₂₋₆ alkenyl, aryl-C₂₋₆ alkenyl, heterocyclyl-C₂₋₆ alkenyl, heteroaryl-C₂₋₆ alkenyl, C₂₋₆ alkylthio wherein the sulfur atom is optionally oxidized to a sulfoxide or sulfone, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH, —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino; or
R⁸ and R⁹ can be taken together to form a cycloalkyl or heteraryl ring, with the proviso that it can not be morpholine;
R¹⁰ and R¹¹ are independently selected from hydrogen, C₁₋₆ alkoxy, hydroxy, halo, C₁₋₆ alkyl, C₃₋₇ cycloalkyl;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆ alkyl,
R¹³ is optionally present and, if present, is selected from from hydrogen, C₁₋₆ alkyl, or a halo group
or the pharmaceutically acceptable salts thereof.

6. The compound according to claim 5 wherein:
X is C or N;
R¹ is hydrogen;
R² is optionally present and, if present, is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆alkoxy, C₂₋₆alkenyloxy, C₂₋₆alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is hydrogen or C₁₋₆alkyl;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
R⁶ is hydrogen or C₁₋₆ alkyl, optionally partially or fully halogenated, or R⁵ and R⁶, may optionally be taken together to form a C₃₋₇ cycloalkyl;
R⁷ is hydrogen or C₁₋₆ alkoxy;
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, C₁₋₆ alkyl, piperidinyl, phenyl and heteroaryl, wherein each phenyl, piperidinyl or heteroaryl may be singly or multiply substituted with R^{y}, wherein R^{y} may be chosen from:
halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
R¹³ is optionally present and, if present, is selected from from hydrogen, C₁₋₆ alkyl, or a halo group.

7. The compound according to claim 6 wherein:
X is C or N;
R¹ is hydrogen;
R² is optionally present and, if present, is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆ alkoxy, C₂₋₆ alkenyloxy, C₂₋₆ alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is hydrogen or C₁₋₆alkyl;
R⁵ is optionally present and, when present, is hydrogen, oxo, C₁₋₆ alkyl, optionally partially or fully halogenated, C₂₋₆ alkenyl, C₂₋₆ alkynyl, C₁₋₆ alkoxy, C₂₋₆ alkenyloxy, C₂₋₆ alkynyloxy, aminoC₁₋₆alkyl, C₁₋₆alkylaminoC₁₋₆alkyl, diC₁₋₆alkylaminoC₁₋₆alkyl, hydroxy, heterocyclylC₁₋₆alkyl, wherein the heterocyclyl is chosen from pyrrolidinyl, piperidinyl, piperazinyl, and morpholinyl, C₃₋₇ cycloalkyl, acyl, ═N—OH or ═N—NHheteroaryl;
R⁶ is hydrogen or C₁₋₆ alkyl, optionally partially or fully halogenated, or
R⁵ and R⁶, may optionally be taken together to form a C₃₋₇ cycloalkyl;
R⁷ is hydrogen or C₁₋₆ alkoxy;
R⁸ is chosen from hydrogen, C₁₋₆ alkyl, C₂₋₆ alkenyl, C₂₋₆ alkynyl, C₁₋₆ alkoxy, or C₃₋₇ cycloalkyl;
R⁹ is chosen from hydrogen, C₁₋₆alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran wherein each phenyl, piperidinyl or heteroaryl may be singly or multiply substituted with R^{y}, wherein R^{y} may be chosen from:
halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂, —C(O)N(R^{a})(R^{b}) where R^{a} and R^{b} are independently selected from H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, or —C(O)NH—N═C—R^{e}, where R^{e} is aryl or heteroaryl optionally substituted with R^{x}, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen;
R¹² is selected from ═S, oxo, C₁₋₆ alkyl, cyano, aminoalkyl, amino, haloalkyl, hydroxyalkyl, ═N—OR^{z}, ═N—N(R^{z})₂, wherein R^{z} is selected from hydrogen or C₁₋₆alkyl; and
R¹³ is optionally present and, if present, is selected from from hydrogen, C₁₋₆ alkyl, or a halo group.

8. The compound according to claim 7 wherein:
X is N;
R¹ is hydrogen;
R³ is hydrogen, C₁₋₆ alkyl, amino C₁₋₆ alkyl, hydroxyC₁₋₆ alkyl, cyano C₁₋₆ alkyl, C₃₋₇ cycloalkyl, carbamoyl or CO₂C₁₋₆alkyl; wherein each alkyl, amino, cycloalkyl or carbamoyl group is optionally substituted by R^{x} wherein R^{x} can be chosen from:
hydrogen, amino, C₁₋₆alkyl, C₂₋₆ alkenyl, amino C₁₋₆ alkyl, hydroxy C₁₋₆ alkyl, cyano C₁₋₆ alkyl, C₂₋₆ alkenyl, C₂₋₆ alkynyl, C₁₋₆alkoxy, C₂₋₆alkenyloxy, C₂₋₆alkynyloxy, hydroxy, C₃₋₇ cycloalkyl, acyl, C₃₋₇ cycloalkyl-C₁₋₆ alkyl, C₃₋₇ cycloalkyl-C₂₋₆ alkenyl, carbonyl, carbamoyl, or —CO₂C₁₋₆alkyl;
R⁴ is optionally present and, when present, is selected from hydrogen, or C₁₋₆alkyl;
R⁵ is optionally present and, when present, is hydrogen, C₁₋₆ alkyl or CF₃;
R⁶ is hydrogen, C₁₋₆ alkyl or CF₃; or
R⁵ and R⁶, may optionally be taken together to form a C₃₋₇ cycloalkyl;
R⁷ is hydrogen;
R⁸ is hydrogen;
R⁹ is chosen from hydrogen, C₁₋₆ alkyl, piperidinyl, phenyl, heteroaryl selected from pyridyl, quinolinyl, thiazolyl, indolyl, benzothiazolyl, benzoxazolyl, isoxazolyl, pyrazinyl, pyrimidinyl, benzimidazolyl, oxazolyl, pyrazolyl, 1,3,4-thiadiazolyl, tetrazolyl, 1,3,4-oxadiazolyl, 1,2,4-triazolyl, imidazolyl, pyrrolyl, thiophene, benzothiophene, furanyl and benzofuran, wherein each phenyl, piperidinyl or heteroaryl may be singly or multiply substituted with R^{y}, wherein R^{y} may be chosen from:
halogen, C₁₋₆ alkyl, C₂₋₆ alkenyl, C₁₋₆ alkoxy, hydroxy, C₃₋₇ cycloalkyl, phenyl, phenoxy, acyl, —OCF₃, amino, C₁₋₃alkylamino, diC₁₋₃alkylamino, acylamino, hydroxyethylamino, —CN, —CO₂C₁₋₃alkyl, —CH₂NH₂ or —C(O)N(R^{a})(R^{b}) where R^{a} and R^{b} are independently selected from
H, C₁₋₃alkyl, —CH₂C(O)NH₂, —(CH₂)ₙOH and —(CH₂)ₙNR^{c}R^{d}, where n=2 or 3 and R^{c} and R^{d} are independently selected from H, C₁₋₃alkyl and C(O)CH₃, and —C(O)NH—N═C—R^{e}, where R^{e} is phenyl, or
R^{a} and R^{b} together with the N they are connected to can form a pyrrolidine, piperazine, piperidine or morpholine ring optionally substituted with one to three groups selected from —CO₂C₁₋₃alkyl, —C(O)NH₂, —OH, C₁₋₃alkyl, amino, C₁₋₃alkylamino and diC₁₋₃alkylamino;
R¹⁰ and R¹¹ are hydrogen; and
R¹² is oxo.

9. A compound selected from:
4-Methyl-1-oxo-2,3,4,9-tetrahydro-1H-β-carboline-6-carboxylic acid phenylamide;
4-Methyl-1-oxo-2,3,4,9-tetrahydro-1H-β-carboline-6-carboxylic acid (4-iodo-phenyl)-amide;
4-Methyl-1-oxo-2,3,4,9-tetrahydro-1H-b-carboline-6-carboxylic acid methylamide;
4-Methyl-1-oxo-2,3,4,9-tetrahydro-1H-β-carboline-6-carboxylic acid (3-methoxy-phenyl)-amide;
4-Methyl-1-oxo-2,3,4,9-tetrahydro-1H-β-carboline-6-carboxylic acid (4-methoxy-phenyl)-amide;
4-Methyl-1-oxo-2,3,4,9-tetrahydro-1H-β-carboline-6-carboxylic acid (3,4-dimethyl-phenyl)-amide;
4-Methyl-1-oxo-2,3,4,9-tetrahydro-1H-β-carboline-6-carboxylic acid indan-5-ylamide;
4-Methyl-1-oxo-2,3,4,9-tetrahydro-1H-β-carboline-6-carboxylic acid p-tolylamide;
4-Methyl-1-oxo-2,3,4,9-tetrahydro-1H-β-carboline-6-carboxylic acid (4-isopropyl-phenyl)-amide;
4,4-spirocyclobutyl-1-oxo-2,3,4,9-tetrahydro-1H-β-carboline-6-carboxylic acid phenylamide;
4,4-spirocyclobutyl-1-oxo-2,3,4,9-tetrahydro-1H-β-carboline-6-carboxylic acid pyridin-4-ylamide;
4,4-spirocyclobutyl-1-oxo-2,3,4,9-tetrahydro-1H-β-carboline-6-carboxylic acid (3-methoxy-phenyl)-amide;
4,4-spirocyclobutyl-1-oxo-2,3,4,9-tetrahydro-1H-β-carboline-6-carboxylic acid naphthalen-2-ylamide;
4,4-spirocyclobutyl-1-oxo-2,3,4,9-tetrahydro-1H-β-carboline-6-carboxylic acid quinolin-3-ylamide;
4,4-spirocyclobutyl-1-oxo-2,3,4,9-tetrahydro-1H-β-carboline-6-carboxylic acid quinolin-6-ylamide;
4,4-spirocyclobutyl-1-oxo-2,3,4,9-tetrahydro-1H-β-carboline-6-carboxylic acid (3-phenoxy-phenyl)-amide;
4,4-spirocyclobutyl-1-oxo-2,3,4,9-tetrahydro-1H-β-carboline-6-carboxylic acid benzo[1,3]dioxol-5-ylamide;
4,4-spirocyclobutyl-1-oxo-2,3,4,9-tetrahydro-1H-β-carboline-6-carboxylic acid pyridin-3-ylamide;
4,4-spirocyclobutyl-1-oxo-2,3,4,9-tetrahydro-1H-β-carboline-6-carboxylic acid (3,4,5-trimethoxy-phenyl)-amide;
1-Oxo-1,2,3,4,5,10-hexahydro-azepino[3,4-β]indole-7-carboxylic acid phenylamide;
4-Methyl-1-oxo-2,3,4,9-tetrahydro-1H-β-carboline-6-carboxylic acid pyridin-4-ylamide;
4-Methyl-1-oxo-2,3,4,9-tetrahydro-1H-β-carboline-6-carboxylic acid (3,5-dichloro-phenyl)-amide;
4-Methyl-1-oxo-2,3,4,9-tetrahydro-1H-β-carboline-6-carboxylic acid (4-chloro-phenyl)-amide;
4-Methyl-1-oxo-2,3,4,9-tetrahydro-1H-β-carboline-6-carboxylic acid pyridin-3-ylamide;
4-Methyl-1-oxo-2,3,4,9-tetrahydro-1H-β-carboline-6-carboxylic acid (3-chloro-phenyl)-amide;
8-Oxo-6,7,8,9-tetrahydro-5H-carbazole-3-carboxylic acid phenylamide;
1-Oxo-1,2,3,4,5,10-hexahydro-azepino[3,4-b]indole-7-carboxylic acid pyridin-3-ylamide;
4-Methyl-1-oxo-2,3,4,9-tetrahydro-1H-β-carboline-6-carboxylic acid quinolin-3-ylamide;
4-Methyl-1-oxo-2,3,4,9-tetrahydro-1H-β-carboline-6-carboxylic acid quinolin-6-ylamide;
4-Methyl-1-oxo-2,3,4,9-tetrahydro-1H-b-carboline-6-carboxylic acid benzo[1,3]dioxol-5-ylamide;
4-Methyl-1-oxo-2,3,4,9-tetrahydro-1H-b-carboline-6-carboxylic acid quinolin-8-ylamide;
4-Methyl-1-oxo-2,3,4,9-tetrahydro-1H-β-carboline-6-carboxylic acid (3-dimethylamino-phenyl)-amide;
4-Methyl-1-oxo-2,3,4,9-tetrahydro-1H-β-carboline-6-carboxylic acid pyridin-2-ylamide;
4-Methyl-1-oxo-2,3,4,9-tetrahydro-1H-β-carboline-6-carboxylic acid (1H-indol-6-yl)-amide;
4-Methyl-1-oxo-2,3,4,9-tetrahydro-1H-β-carboline-6-carboxylic acid quinolin-2-ylamide;
4-Methyl-1-oxo-2,3,4,9-tetrahydro-1H-β-carboline-6-carboxylic acid (3-cyano-phenyl)-amide;
4-Methyl-1-oxo-2,3,4,9-tetrahydro-1H-β-carboline-6-carboxylic acid thiazol-2-ylamide;
4-Methyl-1-oxo-2,3,4,9-tetrahydro-1H-β-carboline-6-carboxylic acid thiazol-2-ylamide;
3-[(4-Methyl-1-oxo-2,3,4,9-tetrahydro-1H-β-carboline-6-carbonyl)-amino]-benzoic acid ethyl ester;
4-[(4-Methyl-1-oxo-2,3,4,9-tetrahydro-1H-β-carboline-6-carbonyl)-amino]-benzoic acid methyl ester;
4-Methyl-1-oxo-2,3,4,9-tetrahydro-1H-β-carboline-6-carboxylic acid (4-carbamoyl-phenyl)-amide;
4-Methyl-1-oxo-2,3,4,9-tetrahydro-1H-β-carboline-6-carboxylic acid (3-carbamoyl-phenyl)-amide;
4,4-Dimethyl-1-oxo-2,3,4,9-tetrahydro-1H-β-carboline-6-carboxylic acid pyridin-3-ylamide;
1-Oxo-2,3,4,9-tetrahydro-1H-β-carboline-6-carboxylic acid pyridin-3-ylamide;
4,4-Dimethyl-1-oxo-2,3,4,9-tetrahydro-1H-β-carboline-6-carboxylic acid thiazol-2-ylamide;
4,4-Dimethyl-1-oxo-2,3,4,9-tetrahydro-1H-β-carboline-6-carboxylic acid (4-carbamoyl-phenyl)-amide;
4-Methyl-1-oxo-2,3,4,9-tetrahydro-1H-β-carboline-6-carboxylic acid [6-(1-methyl-piperidin-4-ylcarbamoyl)-pyridin-3-yl]-amide;
4-Methyl-1-oxo-2,3,4,9-tetrahydro-1H-β-carboline-6-carboxylic acid (2-carbamoyl-phenyl)-amide;
4-Methyl-1-oxo-2,3,4,9-tetrahydro-1H-β-carboline-6-carboxylic acid [6-(3-methylamino-propylcarbamoyl)-pyridin-3-yl]-amide;
4-Methyl-1-oxo-2,3,4,9-tetrahydro-1H-β-carboline-6-carboxylic acid (2-cyano-phenyl)-amide;
4-Methyl-1-oxo-2,3,4,9-tetrahydro-1H-β-carboline-6-carboxylic acid [6-(3-dimethylamino-propylcarbamoyl)-pyridin-3-yl]-amide;
4-Methyl-1-oxo-2,3,4,9-tetrahydro-1H-β-carboline-6-carboxylic acid [6-(2-acetylamino-ethylcarbamoyl)-pyridin-3-yl]-amide;
4-Methyl-1-oxo-2,3,4,9-tetrahydro-1H-β-carboline-6-carboxylic acid [6-(3-amino-propylcarbamoyl)-pyridin-3-yl]-amide;
4-Methyl-1-oxo-2,3,4,9-tetrahydro-1H-β-carboline-6-carboxylic acid {6-[(3-amino-propyl)-methyl-carbamoyl]-pyridin-3-yl}-amide;
4-Methyl-1-oxo-2,3,4,9-tetrahydro-1H-β-carboline-6-carboxylic acid [6-(2-carbamoyl-pyrrolidine-1-carbonyl)-pyridin-3-yl]-amide;
4-Methyl-1-oxo-2,3,4,9-tetrahydro-1H-β-carboline-6-carboxylic acid [6-(4-dimethylamino-piperidine-1-carbonyl)-pyridin-3-yl]-amide;
4-Methyl-1-oxo-2,3,4,9-tetrahydro-1H-β-carboline-6-carboxylic acid [6-(3-dimethylamino-piperidine-1-carbonyl)-pyridin-3-yl]-amide;
4-Methyl-1-oxo-2,3,4,9-tetrahydro-1H-β-carboline-6-carboxylic acid [6-(carbamoyl methyl-carbamoyl)-pyridin-3-yl]-amide;
4-Methyl-1-oxo-2,3,4,9-tetrahydro-1H-β-carboline-6-carboxylic acid (3-bromo-phenyl)-amide;
4-Methyl-1-oxo-2,3,4,9-tetrahydro-1H-β-carboline-6-carboxylic acid (4-methyl-thiazol-2-yl)-amide;
4-Methyl-1-oxo-2,3,4,9-tetrahydro-1H-β-carboline-6-carboxylic acid (4-fluoro-phenyl)-amide;
4-Methyl-1-oxo-2,3,4,9-tetrahydro-1H-β-carboline-6-carboxylic acid (3-trifluoromethoxy-phenyl)-amide;
4-Methyl-1-oxo-2,3,4,9-tetrahydro-1H-β-carboline-6-carboxylic acid (3-acetyl-phenyl)-amide;
4-Methyl-1-oxo-2,3,4,9-tetrahydro-1H-β-carboline-6-carboxylic acid (4-phenoxy-phenyl)-amide;
4-Methyl-1-oxo-2,3,4,9-tetrahydro-1H-β-carboline-6-carboxylic acid (3-phenoxy-phenyl)-amide;
4-Methyl-1-oxo-2,3,4,9-tetrahydro-1H-β-carboline-6-carboxylic acid (4-bromo-phenyl)-amide;
7-Methoxy-4-methyl-1-oxo-2,3,4,9-tetrahydro-1H-β-carboline-6-carboxylic acid pyridin-3-ylamide;
4-Methyl-1-oxo-2,3,4,9-tetrahydro-1H-β-carboline-6-carboxylic acid isoxazol-3-ylamide;
4-Methyl-1-oxo-2,3,4,9-tetrahydro-1H-β-carboline-6-carboxylic acid (4-acetyl-phenyl)-amide;
4-Methyl-1-oxo-2,3,4,9-tetrahydro-1H-β-carboline-6-carboxylic acid (4-acetylamino-phenyl)-amide;
4-Methyl-1-oxo-2,3,4,9-tetrahydro-1H-β-carboline-6-carboxylic acid (3-acetylamino-phenyl)-amide;
4-Methyl-1-oxo-2,3,4,9-tetrahydro-1H-β-carboline-6-carboxylic acid (6-methoxy-pyridin-3-yl)-amide;
4-Methyl-1-oxo-2,3,4,9-tetrahydro-1H-β-carboline-6-carboxylic acid (3-fluoro-phenyl)-amide;
4-Methyl-1-oxo-2,3,4,9-tetrahydro-1H-β-carboline-6-carboxylic acid (4-trifluoromethoxy-phenyl)-amide;
4-Methyl-1-oxo-2,3,4,9-tetrahydro-1H-β-carboline-6-carboxylic acid pyrimidin-4-ylamide;
4-Methyl-1-oxo-2,3,4,9-tetrahydro-1H-β-carboline-6-carboxylic acid pyrazin-2-ylamide;
4-Methyl-1-oxo-2,9-dihydro-1H-β-carboline-6-carboxylic acid pyridin-3-ylamide;
4-Methyl-1-oxo-2,3,4,9-tetrahydro-1H-β-carboline-6-carboxylic acid (3-methyl-isoxazol-5-yl)-amide;
4-Methyl-1-oxo-2,3,4,9-tetrahydro-1H-β-carboline-6-carboxylic acid m-tolylamide;
4-Methyl-1-oxo-2,3,4,9-tetrahydro-1H-β-carboline-6-carboxylic acid (3-ethyl-phenyl)-amide;
4-Methyl-1-oxo-2,3,4,9-tetrahydro-1H-β-carboline-6-carboxylic acid (3-vinyl-phenyl)-amide;
4-Methyl-1-oxo-2,3,4,9-tetrahydro-1H-β-carboline-6-carboxylic acid (6-chloro-pyridin-3-yl)-amide;
4-Methyl-1-oxo-2,3,4,9-tetrahydro-1H-β-carboline-6-carboxylic acid (4-tert-butyl-phenyl)-amide;
4-Methyl-1-oxo-2,3,4,9-tetrahydro-1H-β-carboline-6-carboxylic acid (2-fluoro-phenyl)-amide;
4-Methyl-1-oxo-2,3,4,9-tetrahydro-1H-β-carboline-6-carboxylic acid (3,5-difluoro-phenyl)-amide;
4-Methyl-1-oxo-2,3,4,9-tetrahydro-1H-β-carboline-6-carboxylic acid (3-hydroxymethyl-phenyl)-amide;
4-Methyl-1-oxo-2,3,4,9-tetrahydro-1H-β-carboline-6-carboxylic acid (3-ethynyl-phenyl)-amide;
4-Methyl-1-oxo-2,3,4,9-tetrahydro-1H-β-carboline-6-carboxylic acid pyrimidin-2-ylamide;
4-Methyl-1-oxo-2,3,4,9-tetrahydro-1H-β-carboline-6-carboxylic acid benzothiazol-2-ylamide;
4-Methyl-1-oxo-2,3,4,9-tetrahydro-1H-β-carboline-6-carboxylic acid (3,4-difluoro-phenyl)-amide;
8-Oxo-6,7,8,9-tetrahydro-5H-carbazole-3-carboxylic acid pyridin-3-ylamide;
4-Methyl-1-oxo-2,3,4,9-tetrahydro-1H-β-carboline-6-carboxylic acid (3-tert-butyl-phenyl)-amide;
8-Oxo-6,7,8,9-tetrahydro-5H-carbazole-3-carboxylic acid quinolin-3-ylamide;
4-Methyl-1-oxo-2,3,4,9-tetrahydro-1H-β-carboline-6-carboxylic acid (4-acetylamino-3-cyano-phenyl)-amide;
4-Methyl-1-oxo-2,3,4,9-tetrahydro-1H-β-carboline-6-carboxylic acid (3-isopropyl-phenyl)-amide;
4-Methyl-1-oxo-2,3,4,9-tetrahydro-1H-β-carboline-6-carboxylic acid (3-fluoro-4-hydroxy-phenyl)-amide;
4-Methyl-1-oxo-2,3,4,9-tetrahydro-1H-β-carboline-6-carboxylic acid [3-(1-methyl-cyclopropyl)-isoxazol-5-yl]-amide;
4-Methyl-1-oxo-2,3,4,9-tetrahydro-1H-β-carboline-6-carboxylic acid (4-bromo-3-fluoro-phenyl)-amide;
8-Hydroxyimino-6,7,8,9-tetrahydro-5H-carbazole-3-carboxylic acid pyridin-3-ylamide;
8-Hydroxy-6,7,8,9-tetrahydro-5H-carbazole-3-carboxylic acid pyridin-3-ylamide;
8-Oxo-6,7,8,9-tetrahydro-5H-carbazole-3-carboxylic acid (3-cyano-phenyl)-amide;
4-Methyl-1-oxo-2,3,4,9-tetrahydro-1H-β-carboline-6-carboxylic acid (4-methylcarbamoyl-phenyl)-amide;
4-Methyl-1-oxo-2,3,4,9-tetrahydro-1H-β-carboline-6-carboxylic acid [4-(2-hydroxy-ethylcarbamoyl)-phenyl]-amide;
4-Methyl-1-oxo-2,3,4,9-tetrahydro-1H-β-carboline-6-carboxylic acid [4-(3-hydroxy-propylcarbamoyl)-phenyl]-amide;
4-Methyl-1-oxo-2,3,4,9-tetrahydro-1H-β-carboline-6-carboxylic acid [4-(2-dimethylamino-ethylcarbamoyl)-phenyl]-amide;
4-Methyl-1-oxo-2,3,4,9-tetrahydro-1H-β-carboline-6-carboxylic acid [4-(morpholine-4-carbonyl)-phenyl]-amide;
4-Methyl-1-oxo-2,3,4,9-tetrahydro-1H-β-carboline-6-carboxylic acid [4-(4-carbamoyl-piperidine-1-carbonyl)-phenyl]-amide;
4-Methyl-1-oxo-2,3,4,9-tetrahydro-1H-β-carboline-6-carboxylic acid (2-bromo-phenyl)-amide;
4-Methyl-1-oxo-2,3,4,9-tetrahydro-1H-β-carboline-6-carboxylic acid o-tolylamide;
4-Methyl-1-oxo-2,3,4,9-tetrahydro-1H-β-carboline-6-carboxylic acid [4-(4-methyl-piperazine-1-carbonyl)-phenyl]-amide;
4-Methyl-1-oxo-2,3,4,9-tetrahydro-1H-β-carboline-6-carboxylic acid (4-dimethylcarbamoyl-phenyl)-amide;
4-Methyl-1-oxo-2,3,4,9-tetrahydro-1H-β-carboline-6-carboxylic acid [4-(3-dimethylamino-propylcarbamoyl)-phenyl]-amide;
4-Methyl-1-oxo-2,3,4,9-tetrahydro-1H-β-carboline-6-carboxylic acid (6-chloro-pyrazin-2-yl)-amide;
5-[(4-Methyl-1-oxo-2,3,4,9-tetrahydro-1H-β-carboline-6-carbonyl)-amino]-pyridine-2-carboxylic acid methyl ester;
8-(Methyl-hydrazono)-6,7,8,9-tetrahydro-5H-carbazole-3-carboxylic acid pyridin-3-ylamide;
1-Oxo-2,3,4,5,6,11-hexahydro-1H-azocino[3,4-b]indole-8-carboxylic acid pyridin-3-ylamide;
4-Methyl-1-oxo-2,3,4,9-tetrahydro-1H-β-carboline-6-carboxylic acid (5-bromo-pyridin-3-yl)-amide;
2-Fluoro-4-[(4-methyl-1-oxo-2,3,4,9-tetrahydro-1H-β-carboline-6-carbonyl)-amino]-benzoic acid methyl ester;
4-Methyl-1-oxo-2,3,4,9-tetrahydro-1H-β-carboline-6-carboxylic acid [4-(3-carbamoyl-piperidine-1-carbonyl)-phenyl]-amide;
4-Methyl-1-oxo-2,3,4,9-tetrahydro-1H-β-carboline-6-carboxylic acid [4-(3-hydroxy-piperidine-1-carbonyl)-phenyl]-amide;
4-Methyl-1-oxo-2,3,4,9-tetrahydro-1H-β-carboline-6-carboxylic acid [4-(4-hydroxy-piperidine-1-carbonyl)-phenyl]-amide;
1-{4-[(4-Methyl-1-oxo-2,3,4,9-tetrahydro-1H-β-carboline-6-carbonyl)-amino]-benzoyl}-piperidine-2-carboxylic acid ethyl ester;
1-{4-[(4-Methyl-1-oxo-2,3,4,9-tetrahydro-1H-β-carboline-6-carbonyl)-amino]-benzoyl}-piperidine-3-carboxylic acid ethyl ester;
1-{4-[(4-Methyl-1-oxo-2,3,4,9-tetrahydro-1H-β-carboline-6-carbonyl)-amino]-benzoyl}-piperidine-4-carboxylic acid methyl ester;
3-Hydroxymethyl-1-oxo-2,3,4,9-tetrahydro-1H-β-carboline-6-carboxylic acid pyridin-3-ylamide;
4-Methyl-1-oxo-2,3,4,9-tetrahydro-1H-β-carboline-6-carboxylic acid [4-(3-hydroxy-pyrrolidine-1-carbonyl)-phenyl]-amide;
4-Methyl-1-oxo-2,3,4,9-tetrahydro-1H-β-carboline-6-carboxylic acid [4-(3-dimethylamino-pyrrolidine-1-carbonyl)-phenyl]-amide;
4-Methyl-1-oxo-2,3,4,9-tetrahydro-1H-β-carboline-6-carboxylic acid (6-carbamoyl-pyridin-3-yl)-amide;
4-Methyl-1-oxo-2,3,4,9-tetrahydro-1H-β-carboline-6-carboxylic acid (4-carbamoyl-3-fluoro-phenyl)-amide;
8-Methoxyimino-5,5-dimethyl-6,7,8,9-tetrahydro-5H-carbazole-3-carboxylic acid thiazol-2-ylamide;
5,5-Dimethyl-8-oxo-6,7,8,9-tetrahydro-5H-carbazole-3-carboxylic acid pyridin-3-ylamide;
8-Hydroxyimino-5,5-dimethyl-6,7,8,9-tetrahydro-5H-carbazole-3-carboxylic acid pyridin-3-ylamide;
4-Methyl-1-oxo-2,3,4,9-tetrahydro-1H-β-carboline-6-carboxylic acid (4,5-dimethyl-thiazol-2-yl)-amide;
2-[(4-Methyl-1-oxo-2,3,4,9-tetrahydro-1H-β-carboline-6-carbonyl)-amino]-thiazole-4-carboxylic acid methyl ester;
2-[(4-Methyl-1-oxo-2,3,4,9-tetrahydro-1H-β-carboline-6-carbonyl)-amino]-thiazole-5-carboxylic acid methyl ester;
4-Methyl-1-oxo-2,3,4,9-tetrahydro-1H-β-carboline-6-carboxylic acid (5-methyl-thiazol-2-yl)-amide;
{2-[(4-Methyl-1-oxo-2,3,4,9-tetrahydro-1H-β-carboline-6-carbonyl)-amino]-thiazol-4-yl}-acetic acid ethyl ester;
4-Methyl-1-oxo-2,3,4,9-tetrahydro-1H-β-carboline-6-carboxylic acid (5-acetyl-4-methyl-thiazol-2-yl)-amide;
4-Methyl-2-[(4-methyl-1-oxo-2,3,4,9-tetrahydro-1H-β-carboline-6-carbonyl)-amino]-thiazole-5-carboxylic acid methyl ester;
4-Methyl-1-oxo-2,3,4,9-tetrahydro-1H-β-carboline-6-carboxylic acid (4-cyclopropyl-thiazol-2-yl)-amide;
4-Methyl-1-oxo-2,3,4,9-tetrahydro-1H-β-carboline-6-carboxylic acid (4-phenyl-thiazol-2-yl)-amide;
8-Hydroxyimino-5,5-dimethyl-6,7,8,9-tetrahydro-5H-carbazole-3-carboxylic acid thiazol-2-ylamide;
4-Methyl-1-oxo-2,3,4,9-tetrahydro-1H-β-carboline-6-carboxylic acid [6-(2-dimethylamino-ethylcarbamoyl)-pyridin-3-yl]-amide;
3-Aminomethyl-1-oxo-2,3,4,9-tetrahydro-1H-β-carboline-6-carboxylic acid thiazol-2-ylamide;
3-Aminomethyl-1-oxo-2,3,4,9-tetrahydro-1H-β-carboline-6-carboxylic acid quinolin-3-ylamide;
8-Hydrazono-5,5-dimethyl-6,7,8,9-tetrahydro-5H-carbazole-3-carboxylic acid pyridin-3-ylamide;
4-Methyl-1-oxo-2,3,4,9-tetrahydro-1H-β-carboline-6-carboxylic acid (4-tert-butyl-thiazol-2-yl)-amide;
4-Methyl-1-oxo-2,3,4,9-tetrahydro-1H-β-carboline-6-carboxylic acid (4,5,6,7-tetrahydro-benzothiazol-2-yl)-amide;
8-Amino-6,7,8,9-tetrahydro-5H-carbazole-3-carboxylic acid pyridin-3-ylamide;
8-Hydroxyimino-6,7,8,9-tetrahydro-5H-carbazole-3-carboxylic acid pyridin-3-ylamide;
8-[(Z)-Hydroxyimino]-6,7,8,9-tetrahydro-5H-carbazole-3-carboxylic acid pyridin-3-ylamide;
3-Cyanomethyl-1-oxo-2,3,4,9-tetrahydro-1H-β-carboline-6-carboxylic acid pyridin-3-ylamide;
3-Aminomethyl-1-oxo-2,3,4,9-tetrahydro-1H-β-carboline-6-carboxylic acid pyridin-3-ylamide;
4-Methyl-1-oxo-2,3,4,9-tetrahydro-1H-β-carboline-6-carboxylic acid (4-carbamoyl-thiazol-2-yl)-amide;
4-Methyl-1-oxo-2,3,4,9-tetrahydro-1H-β-carboline-6-carboxylic acid [4-(2-dimethylamino-ethylcarbamoyl)-thiazol-2-yl]-amide;
4-Methyl-1-oxo-2,3,4,9-tetrahydro-1H-β-carboline-6-carboxylic acid (6-fluoro-benzothiazol-2-yl)-amide;
4-Methyl-1-oxo-1,3,4,9-tetrahydro-p-carboline-2,6-dicarboxylic acid 2-amide 6-pyridin-3-ylamide;
1-[(Z)-Hydroxyimino]-4-methyl-2,3,4,9-tetrahydro-1H-β-carboline-6-carboxylic acid pyridin-3-ylamide;
1-[(Z)-Methoxyimino]-4-methyl-2,3,4,9-tetrahydro-1H-β-carboline-6-carboxylic acid pyridin-3-ylamide;
4-Methyl-1-oxo-2,3,4,9-tetrahydro-1H-β-carboline-6-carboxylic acid (4-aminomethyl-phenyl)-amide;
8-Acetylamino-6,7,8,9-tetrahydro-5H-carbazole-3-carboxylic acid pyridin-3-ylamide;
4-Methyl-1-oxo-2,3,4,9-tetrahydro-1H-β-carboline-6-carboxylic acid (6-bromo-benzothiazol-2-yl)-amide;
4-Methyl-1-oxo-2,3,4,9-tetrahydro-1H-β-carboline-6-carboxylic acid [5-(2-hydroxy-ethylcarbamoyl)-4-methyl-thiazol-2-yl]-amide;
4-Methyl-1-oxo-2,3,4,9-tetrahydro-1H-β-carboline-6-carboxylic acid (3-aminomethyl-phenyl)-amide;
4-Methyl-1-oxo-2,3,4,9-tetrahydro-1H-β-carboline-6-carboxylic acid [4-(3-dimethylamino-propylcarbamoyl)-thiazol-2-yl]-amide;
3-(Acetylamino-methyl)-1-oxo-2,3,4,9-tetrahydro-1H-b-carboline-6-carboxylic acid quinolin-3-ylamide;
8-Ureido-6,7,8,9-tetrahydro-5H-carbazole-3-carboxylic acid pyridin-3-ylamide;
4-Methyl-1-oxo-2,3,4,9-tetrahydro-1H-β-carboline-6-carboxylic acid [4-(2-dimethylamino-ethylcarbamoyl)-3-fluoro-phenyl]-amide;
1,5-Dioxo-1,2,3,4,5,10-hexahydro-azepino[3,4-β]indole-7-carboxylic acid pyridin-3-ylamide;
4-Methyl-1-oxo-2,3,4,9-tetrahydro-1H-β-carboline-6-carboxylic acid (4-methylcarbamoyl-thiazol-2-yl)-amide;
4-Methyl-1-oxo-2,3,4,9-tetrahydro-1H-β-carboline-6-carboxylic acid (4-dimethylcarbamoyl-thiazol-2-yl)-amide;
3-Aminomethyl-1-oxo-2,3,4,9-tetrahydro-1H-β-carboline-6-carboxylic acid pyrazin-2-ylamide;
1-Oxo-5-(pyridin-2-yl-hydrazono)-1,2,3,4,5,10-hexahydro-azepino[3,4-β]indole-7-carboxylic acid pyridin-3-ylamide;
5-Hydroxyimino-1-oxo-1,2,3,4,5,10-hexahydro-azepino[3,4-β]indole-7-carboxylic acid phenylamide;
4-Methyl-1-oxo-2,3,4,9-tetrahydro-1H-β-carboline-6-carboxylic acid (5-carbamoyl-thiazol-2-yl)-amide;
4-Methyl-1-oxo-2,3,4,9-tetrahydro-1H-β-carboline-6-carboxylic acid [5-(2-dimethylamino-ethylcarbamoyl)-thiazol-2-yl]-amide;
3-Aminomethyl-1-oxo-2,3,4,9-tetrahydro-1H-β-carboline-6-carboxylic acid [4-(2-dimethylamino-ethylcarbamoyl)-thiazol-2-yl]-amide;
{6-[4-(2-Dimethylamino-ethylcarbamoyl)-thiazol-2-ylcarbamoyl]-1-oxo-2,3,4,9-tetrahydro-1H-β-carbolin-3-ylmethyl}-carbamic acid ethyl ester; and
4-Methyl-1-oxo-2,3,4,9-tetrahydro-1H-β-carboline-6-carboxylic acid [5-(3-dimethylamino-propylcarbamoyl)-thiazol-2-yl]-amide.

10. A pharmaceutical composition comprising a pharmaceutically effective amount of a compound according to claim 1 and one or more pharmaceutically acceptable carriers and/or adjuvants.

11. A pharmaceutical composition comprising a pharmaceutically effective amount of a compound according to claim 5 and one or more pharmaceutically acceptable carriers and/or adjuvants.

12. A method of treating a cytokine mediated disease or condition which comprises administering to a patient in need of such treatment a therapeutically effective amount of a compound according to claim 1.

13. The method according to claim 11 wherein cytokine mediated disease or condition is selected from rheumatoid arthritis, inflammatory bowel disease, septic shock, osteoarthritis, Crohn's disease, ulcerative colitis, multiple sclerosis, Guillain-Barre syndrome, psoriasis, graft versus host disease, systemic lupus erythematosus, restenosis following percutaneous transluminal coronary angioplasty, diabetes, toxic shock syndrome, Alzheimer's disease, acute and chronic pain, contact dermatitis, atherosclerosis, traumatic arthritis, glomerulonephritis, reperfusion injury, sepsis, bone resorption diseases, chronic obstructive pulmonary disease, congestive heart failure, asthma, stroke, myocardial infarction, thermal injury, adult respiratory distress syndrome (ARDS), multiple organ injury secondary to trauma, dermatoses with acute inflammatory components, acute purulent meningitis, necrotizing enterocolitis and syndromes associated with hemodialysis, leukopherisis and granulocyte transfusion.

14. A method of treating a cytokine mediated disease or condition which comprises administering to a patient in need of such treatment a therapeutically effective amount of a compound according to claim 5.

15. The method according to claim 14 wherein cytokine mediated disease or condition is selected from rheumatoid arthritis, inflammatory bowel disease, septic shock, osteoarthritis, Crohn's disease, ulcerative colitis, multiple sclerosis, Guillain-Barre syndrome, psoriasis, graft versus host disease, systemic lupus erythematosus, restenosis following percutaneous transluminal coronary angioplasty, diabetes, toxic shock syndrome, Alzheimer's disease, acute and chronic pain, contact dermatitis, atherosclerosis, traumatic arthritis, glomerulonephritis, reperfusion injury, sepsis, bone resorption diseases, chronic obstructive pulmonary disease, congestive heart failure, asthma, stroke, myocardial infarction, thermal injury, adult respiratory distress syndrome (ARDS), multiple organ injury secondary to trauma, dermatoses with acute inflammatory components, acute purulent meningitis, necrotizing enterocolitis and syndromes associated with hemodialysis, leukopherisis and granulocyte transfusion.
